(19) United States
(12) Patent Application Publication
(10) Pub. No.: US 2020/0276246 A1
(43) Pub. Date: Sep. 3, 2020

(54) THERAPEUTIC USES OF FLAP OF GENETICALLY MODIFIED CELLS

(71) Applicant: HOLOSTEM TERAPIE AVANZATE S.R.L., Modena (MO) (IT)

(72) Inventors: Michele DE LUCA, Modena (MO) (IT); Graziella PELLEGRINI, Modena (MO) (IT)

(73) Assignee: HOLOSTEM TERAPIE AVANZATE S.R.L., Modena (MO) (IT)

(21) Appl. No.: 16/647,565

(22) PCT Filed: Sep. 18, 2018
(86) PCT No.: PCT/EP2018/075186

§ 371 Date: Mar. 16, 2020

(30) Foreign Application Priority Data
Sep. 19, 2017 (IT) 102017000104587

(51) Int. Cl. A61K 35/36; C12N 5/071; A61P 17/02

(52) U.S. Cl. CPC A61K 35/36; C12N 5/0625; C12N 2533/56; A61P 17/02; C12N 5/0629

(57) ABSTRACT

The present invention refers to a flap of genetically modified cells on fibrin substrate for use in the treatment of Epidermolysis Bullosa (EB) and/or for use in a method to promote in vivo cell adhesion and/or in vivo cell growth and/or cell regeneration and/or for use in a surgical method, preferably for use in the repair or replacement of living tissue, in an EB patient.

### FIELD OF THE INVENTION

The present invention refers to regenerative medicine field. In particular it refers to a flap of genetically modified cells on fibrin substrate for use in the treatment of Epidermolysis Bullosa (EB) and/or for use in a method to promote in vivo cell adhesion and/or in vivo cell growth and/or cell regeneration and/or for use in a surgical method, preferably for use in the repair or replacement of living tissue, in an EB patient.

### BACKGROUND ART

Epidermolysis Bullosa is a rare genetic pathology characterized by mutations of hemidesmosome and/or anchoring fibril proteins. Four big categories of EB exists, distinguished by the rupture site inside dermo-epidermal junction: simple EB (EBS), junctional EB (JEB), dystrophic EB (DAB) and Kindler syndrome (Fine J D. 2010. Inherited epidermolysis bullosa: recent basic and clinical advances. Curr Opin Pediatr 22:453-458). Generalized Junctional Epidermolysis Bullosa (JEB) is a severe, often lethal genetic disease characterized by structural and mechanical fragility of the integuments. Skin and mucosal blisters and erosions occur within the lamina lucida of the basement membrane upon minor trauma. Massive chronic skin and mucosa wounds greatly impair the patients' quality of life, lead to recurrent infections and scars and are predisposing to skin cancer. JEB is caused by mutations in LAMA3, LAMB3 or LAMC2 genes, which jointly encode laminin-332 (a heterotrimeric protein, also known as laminin 5, consisting of α3, β3, and γ2 chains) and in genes encoding collagen XVII and α6β4 integrins¹. Deleterious mutations causing absence of laminin-332 are usually early lethal. In nonlethal JEB, laminin-332 is strongly reduced and hemidesmosomes are rudimentary or absent. There is no cure for JEB and >40% of the patients succumb to the disease by adolescence^{1,2}. Available symptomatic treatments can only relieve the devastating clinical manifestations.

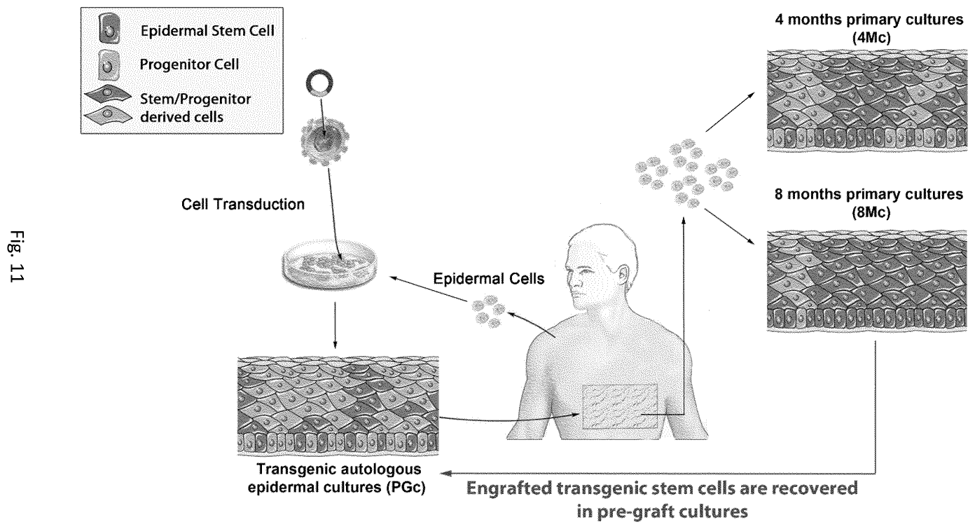

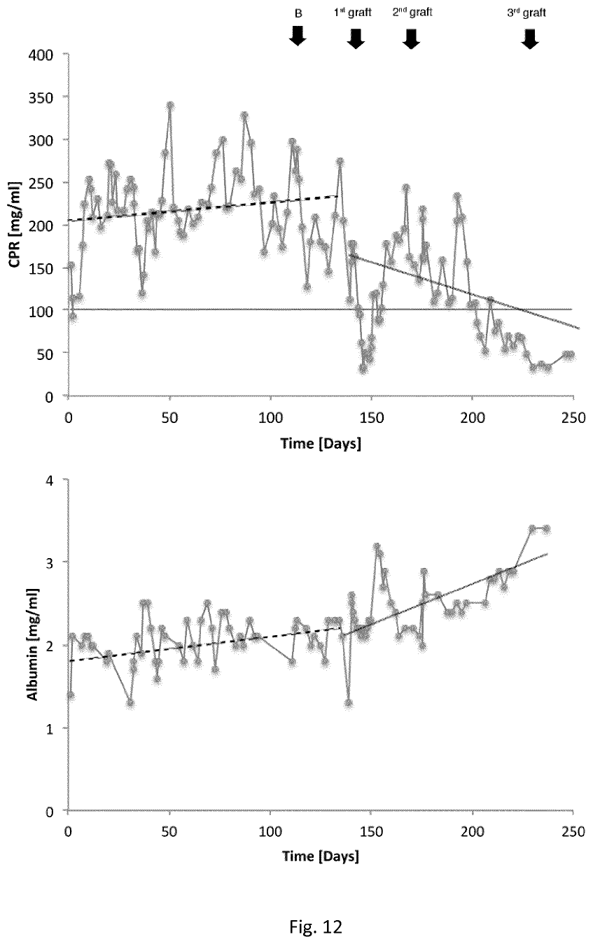

Monthly renewal and timely repair of human epidermis is sustained by epidermal stem cells, which generate colonies known as holoclones^{3,4}. Holoclones produce meroclone- and paraclone-forming cells, which behave as transient amplifying (TA) progenitors^{3,4}. Epithelial cultures harbouring holoclone-forming cells can permanently restore massive skin and ocular defects⁵⁻⁹. A phase I/II clinical trial (1 patient) and a single-case study provided compelling evidence that local transplantation of transgenic epidermal cultures can generate a functional epidermis, leading to permanent (the longest follow-up being of 12 years) correction of JEB skin lesions¹⁰⁻¹². However, paucity of treated areas (a total of ˜0.06 m²) did not significantly improve patients' quality of life¹⁰⁻¹². A major criticism to this therapeutic approach has been its supposed unsuitability for the massive skin lesions marking generalized JEB. To date, the procedure for the preparation of ex vivo genetically modified epidermis flaps involves the culture of cells on plastic supports with the aim of obtaining a genetically modified flap of the epidermis. The procedure described to date, for example in Mavilio et al. 2006⁽¹²⁾ and Bauer et al. 2017⁽¹⁰⁾, consists in plating, on plastic supports of 75 cm²-175 cm², keratinocytes genetically corrected with a retroviral MVL derived vector containing the beta 3 chain of laminin 5, on feeder layers and allowing them to grow and reach full confluence (9-14 days). The attainment of the confluence represents a fundamental step to ensure the stability of the flap (FIG. 13). The reason is due to the intrinsic stratification/differentiation process in keratinocytes, which, once they reach the confluence, slow down their proliferation in favor of stratification/differentiation processes. The stratification process ensures greater stability and compactness on the flap of the epidermis so formed, thus ensuring better maneuverability, a condition necessary for the assembly and transportation phases. Upon reaching the confluence (FIG. 13), the epidermis flap is washed with a solution containing DMEM, L-Glutamine. Subsequently, the flap is dissociated from the plastic support by the addition of Dispase II (2.5 mg/ml). On the upper side (opposite to the one adhering to plastic) a Vaseline® Petrolatum gauze of 50 cm² is applied, which will be fixed to the epidermis flap by clips. Once the flap is secured, this is transferred to a transport flap container (or transportation box)(FIG. 14).

The method for obtaining a flap starting from a plastic support is a long and complicated procedure. There are several steps that may invalidate the release of the same. The following table show the main steps that may lead to the non-conformity of the flap and to the loss of release (Table 3).

TABLE 3
Parameters of non-conformity in releasing
the flap for the transplant.

| | Not |
|---|---|
| Parameters | conform |
| No conform evaluation of culture confluence before detachment | x |
| Presence of breaks after DISPASE detachment | x |
| Presence of breaks after application of gauzes and clips | x |
| Presence of bubbles in the transportation box | x |
| Presence of breaks after shipment | x |
| Breaks during the box transportation opening | x |
| Time of transplant greater than 24 h from the detachment in | x |
| dispase | |

Regardless of the procedure used for preparing the flap, another step that can be a cause of failure to release the flap is its breakup during the transportation phases. Although the flap is secured and locked on the gauze, unintentional movements during transport may cause its breakage or the winding on itself. Indeed, before proceeding with the transplant in operating room, the epidermal flaps genetically corrected are extracted from the transport container and analyzed by visual inspection for the presence of any breaks. In case of breakage, the flap is considered to be inadequate (Table 3).

The procedure described above is not intuitive and without risk. In fact, in the setup phases, given the multiple steps and continuous manipulations, the risk of contamination or the presence of air bubbles, which can alter the O₂ exchange, may result in poor product quality. It is also known that keratinocytes in the absence of adhesion induce the activation of terminal differentiation processes (Watt F M, Jordan P W, O'Neill C H. 1988. Cell shape controls terminal differentiation of human epidermal keratinocytes. Proc Natl Acad Sci USA 85:5576-5580).

In order to exclude an accelerated terminal differentiation due to the loss of contact with substrate, the transplant must be carried out within 24 hours from the detachment and preparation of the flap.

In fact, the stability of the genetically modified flap generated from plastic supports is 24 h. The biological quality as well as the performance of the flap so produced are then remarkably reduced. This represents a great disadvantage as the transplant may be carried out also in faraway countries. Therefore, is still felt the need of providing a flap of genetically modified cells wherein the cells are not subjected to an accelerated terminal differentiation due to the loss of contact with the substrate and which are suitable for the epidermal transplant in EB patients.

The patent application WO2005028638 refers to a process for producing a cell sheet, comprising culturing cells up to a state of saturation on the surface of a support having its surface coated with fibrin, continuing the culturing for a period of time sufficient to achieve decomposition of the fibrin at cell bottom surface and detaching the cultured cells in the form of a sheet from the support surface. Therefore, the patent application WO2005028638 teaches to obtain a sheet of cells not genetically modified, wherein the fibrin is not present because it was previously degraded. Pellegrini et al. 1999⁽⁶⁾, show the potential use of a matrix of fibrin for culturing human epithelial staminal cells for the autologous epidermal transplant in patient with third degree burns on more than 80% of the body. Said publication shows that the culture of human keratinocytes on fibrin doesn't alter the biological properties of the cells and maintains its characteristic of staminality, as demonstrated by the presence of isolated holoclones in these conditions (epidermal stem cells) and by the follow up in patients treated for severe burns (^{5, 6}; Cuono C, Langdon R, McGuire J. 1986. Use of cultured epidermal autografts and dermal allografts as skin replacement after burn injury. Lancet 1:1123-1124; De Luca M, Albanese E, Bondanza S, Megna M, Ugozzoli L, Molina F, Cancedda R, Santi P L, Bormioli M, Stella M, et al. 1989. Multicentre experience in the treatment of burns with autologous and allogenic cultured epithelium, fresh or preserved in a frozen state. Burns 15:303-309).

In 2010, another work was published that demonstrates the clinical effectiveness of transplant of Corneal limbal cell in the treatment of severe burns by corneal epithelium⁽⁸⁾. In both papers, the epithelium was cultivated on a fibrin matrix starting from raw materials (fibrinogen and thrombin) produced, for example, by Baxter (Tissucol). This fibrin has been used in more than 200 epithelial corneal cell transplants, none of which has been found to have any adverse events due to rejection or inflammation. Preferably, the fibrin matrix is produced by Holostem Advanced Therapies, from raw materials (fibrinogen and thrombin) produced for example by Kedrion. A comparative study performed on corneal limbal epithelial cells showed the equivalence of the two products (Table 4).

TABLE 4
Table 4: Resumes the results obtained from a comparative study
carried out starting from different fibrin lots, using excipient
(fibrin and fibrinogen) produced by Baxter (Tissucol) and
by Kedrion. The results obtained show the equivalence of both
products, as evidenced by chlonogenic values (% CFE) almost
unchanged and by the value of the percentage of p63 positive
cells superior when using excipients from Kedrion.

| | |
|---|---|
| lots of TISSUCOL | lots of KEDRION |

| parameter | average | dev stand. | average | dev stand. |
|---|---|---|---|---|
| % CFE | 19.1 | 8.1 | 17.6 | 7.9 |
| % Ab | 14.3 | 4.3 | 20.6 | 12.3 |
| % K3 | 86.9 | 2.4 | 86.7 | 5.4 |
| % K19 | 15.5 | 16.3 | 52.7 | 25.8 |
| % p63 | 1.3 | 0.9 | 2.2 | 1 |

Sheets of cells on a fibrin substrate are therefore already described. However, flaps of genetically modified cells on a fibrin substrate which may be useful in the treatment of EB were not previously disclosed.

It is still felt the need of providing flaps of genetically modified cells suitable to be used in the treatment of EB that overcome the disadvantages of prior art cell sheets prepared on plastic support. Indeed, plastic-cultured grafts need to be enzymatically detached by dispase and mounted on a non-adhering gauze for shipping and handling by the surgeon. There are several disadvantages associated with this method: i) the detached epithelium shrinks by 50% or more of its original size; ii) cells do not retain clonogenic ability for more than 24 hours, limiting long-distance transportation and prohibiting any delay between detachment of the cultures and the time of grafting, iii) during transportation, the epidermis often detaches from the clips needed to anchor it to the gauze, making application on the wound bed quite cumbersome.

### SUMMARY OF THE INVENTION

Inventors have found that a flap of genetically modified cells on a fibrin support overcome the above disclosed disadvantages presented by genetically modified cells cultivated on a plastic support and may be successful used in the treatment of EB, in particular of JEB.

Here inventors show life-saving regeneration of virtually the entire epidermis (˜0.85 m²) on a 7-year-old child suffering from a devastating form of JEB by means of autologous transgenic keratinocyte cultures. The regenerated epidermis remained robust, resistant to mechanical stress and did not develop blisters or erosions during 21 months follow-up. Such fully functional epidermis is entirely sustained by a limited number of transgenic epidermal stem cells, detected as holoclones, able to extensively self-renew in vitro and in vivo.

The proviral integration pattern was maintained in vivo and epidermal renewal did not cause any clonal selection. Clonal tracing showed that human epidermis is not sustained by equipotent progenitors, but by a limited number of long-lived stem cells, detected as holoclones, able to extensively self-renew in vitro and in vivo and to produce progenitors that replenish terminally differentiated keratinocytes.

Keratinocytes cultured on a fibrin matrix have the same growth capacity and stem cell content as those cultured on plastic, but enzymatic detachment and shrinking of the epithelium are avoided. Thus, the same number of clonogenic cells can generate a fibrin-graft at least twice as big as the one made on plastic. Fibrin permits a reduction in the minimum time between biopsy and graft preparation from the previous value of 21 days or more to 16-17 days. Part of this reduction is due to the possibility of using sub-confluent cultures. This is not possible for enzymatically-detached cultures, each of which must consist of a single coherent sheet, since otherwise the detached culture would disintegrate into individual colonies. This would also give more flexibility in planning the surgery. Cultures that are attached to and spread on the fibrin matrix preserve clonogenic ability for at least two days after packaging, further increasing that flexibility. to allow cultured keratinocytes to engraft on the prepared wound bed, fibrin must be degraded within few hours after transplantation. Inventors also showed that fibrin properly degraded in wound beds of epidermolysis bullosa.

### DETAILED DESCRIPTION OF THE INVENTION

It is therefore an object of the invention a flap of genetically modified cells on fibrin substrate for use in the treatment of Epidermolysis Bullosa (EB) wherein said genetically modified cells are genetically modified with at least one heterologous nucleic acid comprising a nucleotide sequence encoding:
a) at least one chain selected from the group consisting of: β3, α3 and γ2 chain of laminin-332, and/or
b) collagen XVII and/or
c) at least one α6β4 integrin and/or
d) collagen VII and/or
e) keratin 5 and/or Keratin 14 and/or
f) Plectin.

A further object of the invention is a flap of genetically modified cells on fibrin substrate for use in a method to promote in vivo cell adhesion and/or in vivo cell growth and/or cell regeneration and/or for use in a surgical method, preferably for use in the repair or replacement of living tissue, in an EB patient wherein said genetically modified cells are genetically modified with at least one heterologous nucleic acid comprising a nucleotide sequence encoding:
a) at least one chain selected from the group consisting of: β3, α3 and γ2 chain oflaminin-332, and/or
b) collagen XVII and/or
c) at least one α6β4 integrin and/or
d) collagen VII and/or
e) keratin 5 and/or Keratin 14 and/or
f) Plectin.

Preferably the EB is Junctional Epidermolysis Bullosa (JEB).

Also object of the invention is a flap of genetically modified cells on fibrin substrate for medical use wherein said genetically modified cells are genetically modified with at least one heterologous nucleic acid comprising a nucleotide sequence encoding:
a) at least one chain selected from the group consisting of: β3, α3 and γ2 chain of laminin-332, and/or
b) collagen XVII and/or
c) at least one α6β4 integrin and/or
d) collagen VII and/or
e) keratin 5 and/or Keratin 14 and/or
f) Plectin.

Preferably the genetically modified are transduced with a gene or a cDNA coding for the protein(s) defined above. Preferably said genetically modified cells are transduced with a gene or cDNA selected from the group consisting of:
a) at least one chain selected from the group consisting of: beta-3, α3 and γ2 chain of laminin-5, and/or
b) collagen 17 and/or
c) at least one α6β4 integrin and/or
d) collagen 7 and/or
e) keratin 5 and Keratin 14 and/or
f) Plectin.

Preferably said genetically modified cells are transduced with a gene or cDNA selected from the group consisting of: beta-3 chain of laminin 5, collagen 7 and collagen 17.

The heterologous nucleic acid preferably comprises a nucleotide sequence encoding laminin-332 β3 chain and/or collagen XVII.

In a preferred embodiment, the gene or cDNA encode for the above-mentioned protein or for an amino acid sequence having at least 75% amino acid sequence identity to the amino acid sequence SEQ ID NO: 6 and/or to the amino acid sequence SEQ ID NO:4 and/or to the amino acid sequence SEQ ID NO: 2.

In a preferred embodiment,
a) the laminin-332 β3 chain comprises an amino acid sequence having at least 75% amino acid sequence identity to the amino acid sequence SEQ ID NO: 6 and/or
b) the collagen XVII comprises an amino acid sequence having at least 75% amino acid sequence identity to the amino acid sequence SEQ ID NO:4 and/or
c) the collagen VII comprises an amino acid sequence having at least 75% amino acid sequence identity to the amino acid sequence SEQ ID NO: 2.

Preferably, the genetically modified cells are cells that have been transduced with a retroviral vector, said retroviral vector preferably being an alpharetroviral vector, a gammaretroviral vector, a lentiviral vector or a spumaretroviral vector.

Said heterologous nucleic acid preferably further comprises a promoter that is operably linked to the promoter, and/or wherein the promoter is heterologous to the encoding nucleotide sequence as defined above and/or said heterologous nucleic acid is under the control of virus long terminal repeat (LTR), preferably of retrovirus LTR, more preferably of Moloney Leukaemia virus (MLV) LTR.

Said genetically modified cells preferably have been transduced with the at least one heterologous nucleic acid as defined above.

The transduction was preferably carried out with a viral vector, preferably with a retroviral vector, said retroviral vector preferably being an alpharetroviral vector, a gammaretroviral vector, a lentiviral vector or a spumaretroviral vector.

In a preferred embodiment of the invention the flap as above defined is obtainable by an in vitro method, characterized by:
a) plating feeder cells on the upper surface of a fibrin substrate so as to obtain a fibrin substrate on which said feeder cells are adhered;
b) plating and cultivating to subconfluence said genetically modified cells on said fibrin substrate onto which feeder cells are adhered, said fibrin substrate being positioned on a solid support so that the cells do not interact with the surface of said support so as to obtain a flap of genetically modified cells adhered to said fibrin substrate;
c) detaching the flap of genetically modified cells adhered to said fibrin substrate from the support in a form similar to a sheet to obtain a flap of genetically modified cells on fibrin substrate.

Said solid support is preferably of plastic, e.g. a Petri dish, or of glass.

Said feeder cells are preferably plated on the fibrin substrate from 2 to 24 hours before plating the genetically modified cells.

Preferably the above method further comprises:
before step c), the steps:
b′) removing the culture medium and/or
b″) washing the flap of genetically modified cells adhered to said fibrin substrate with a washing solution
and/or after step c), the step of:
d) placing the obtained flap of genetically modified cells on fibrin substrate in a transport container Said fibrin substrate has preferably dimensions of from about 0.32 cm² to about 300 cm², preferably of about 31-144 cm², more preferably of 144 cm².

The transport container preferably comprises a transport medium.

Preferably, said fibrin substrate comprises from about 20 to about 100 mg/ml of fibrinogen and from about 1 to about 10 IU/ml of thrombin. More preferably, said fibrin substrate comprises from about 20 to about 50 mg/ml of fibrinogen, preferably from about 20 to about 40 mg/ml of fibrinogen, and from about 3 to about 8 IU/ml of thrombin; even more preferably it comprises from about 20 to about 25 mg/ml of fibrinogen and from about 2 to about 4 IU/ml of thrombin. In a preferred aspect said fibrin substrate comprises about 23.1 mg/ml of fibrinogen and about 3.1 IU/ml of thrombin.

Preferably, said genetically modified cells are epithelial cells, preferably primary epithelial cells deriving from stratified epithelia, more preferably epidermal cells, preferably keratinocytes, more preferably human primary keratinocytes isolated from biopsies, preferably cutaneous biopsies.

Preferably the cutaneous biopsies are isolated from a EB patient, preferably a JEB patient, said EB patient preferably being the same patient subject to the treatment.

In a preferred embodiment of the invention above disclosed, thawed genetically modified cells, in particular keratinocytes cells, and feeder cells may be plated at the same time. Alternatively, it is possible to plate feeder cells and after 2 h-24 h thawing the genetically modified cells, in particular keratinocytes.

In a preferred aspect of the invention, the method consists in plating genetically correct keratinocytes and feeder layers onto a fibrin matrix (or substrate) of the size of 144 cm².

It is also an object of the invention a method for the treatment and/or prevention of Epidermolysis Bullosa (EB) comprising administering to a subject the flap of genetically modified cells on fibrin substrate as above defined. The administration is e.g. carried out by applying or transplanting transgenic epidermal grafts on the defective body surface, preferably on a properly prepared dermal wound bed. The application of the grafts is preferably carried out sequentially.

It is also an object of the invention the use of the flap of genetically modified cells on fibrin substrate as above defined for the manufacture of a medicament, in particular for treating Epidermolysis Bullosa (EB).

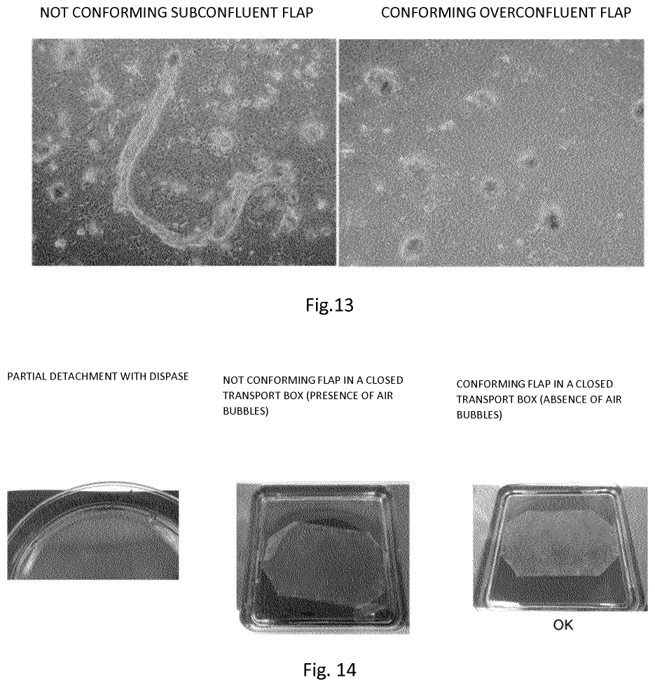

Unlike the production process of the epidermis on plastic supports, the keratinocytes cultivated under these conditions do not have to reach full confluence, but the subconfluence to proceed with the preparation of this for transport (FIG. 15).

In the context of the present invention, the expression “flap of genetically modified cells on fibrin substrate” includes flap of cells that were grown on a fibrine substrate or on feeder cells grown on a fibrine substrate.

Fibrin provides growth support to keratinocytes, both in the transport phase and before detaching from the support, and also in the first transplant phases, thus securing a high proliferative/regenerative potential of the keratinocytes. This prevents an accelerated differentiation process due to contact loss, found in the epidermis flap derived from plastic growth (Table 3). During the transport phases, post separation of the flap (including cells, for example, epidermis, and fibrin) and during the first transplant phases, cells, particularly keratinocytes, will complete their growth and begin the in vivo layering/differentiation process.

Despite the greater flexibility and handling of fibrin in the transport and transplant phase, it is still necessary to perform the compliance checks before the release of the flap. As shown in FIG. 15, holes in the fibrin or disomogeneity in the keratinocyte or feeder plating make the flap non-conforming to release.

Fibrin is an ideal support for the growth of keratinocytes because it represents a compact and solid biodegradable biological matrix that ensures a great deal of maneuverability during preparation and transport phases.

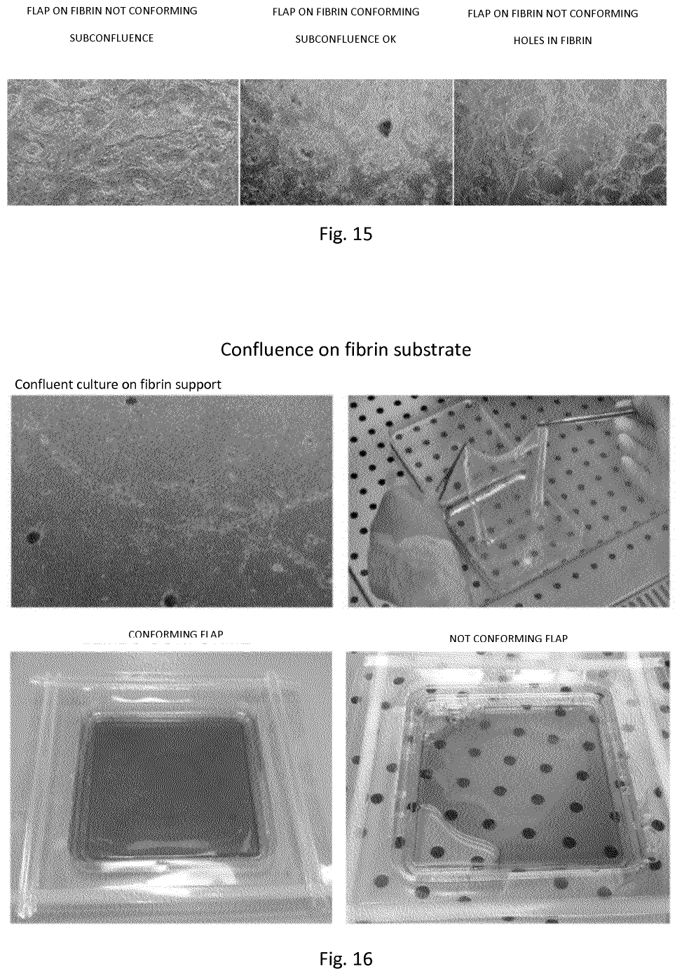

The fibrin flap obtained is washed with a solution containing DMEM and L-Glutamine. Then, by means of sterile pliers, it is detached from the holder and placed in the transport container (FIG. 16), where the transport medium will be added. The container is then sealed ensuring that no air bubble is present. The presence of bubbles would render the flap release not adequate for transplants (FIG. 16).

Unlike the flap derived from plastic growth, the fracture of the flap on the fibrin during the transport phases is a very rare event. The fibrin, once transplanted on the receiving bed, is subjected to a slow and natural degradation in loco due to the fibrinolysis process which allows the direct contact of the genetically modified epidermal flap with the underneath derma. In this way, a natural process of terminal differentiation and stratification is assured.

The transplant of genetically modified epidermis on fibrin support also guarantees the attachment of a greater number of chlonogenic cells and of staminal cells, as evidenced by the CFE derived from flaps cultivated on plastic support and flaps cultivated on fibrin support and a stability of 36 h (Table 5). As can be seen from Table 5, control of the process carried on isolated samples of flaps cultivated on plastic show indeed a reduction of clonogenicity in two different transplants (Table 5). This renders the cultivation on plastic less flexible and manageable for carrying out transplants in different European centers.

TABLE 5
comparative colony forming efficiency of cells isolated from the plastic
and fibrin supports in two different patients. Patient 0101 is the patient
of the publication Bauer J. W. et al. 2014 ⁽³⁷⁾. Lots 0201-0204 refer
to transplants on the patient herein disclosed.

| | CFE drug | CFE drug | CFE drug |
|---|---|---|---|
| Lots | substance | product plastic | product fibrin |
| 0101 | 15% | 1% | — |
| 0201 | 34% | 2% | 20.70% |
| 0202 | 28.50% | — | 35.40% |
| 0203 | 26.80% | — | 22.50% |
| 0204 | 11.50% | — | 17.20% |

In addition, the performance and biological stability of the product are superior if compared to flap resulting from growth on plastic support.

In addition to this, the reduced risk of microbiological contamination due to the small number of manipulations required to set up the flap and the reduced risk of breakages in transport and in the collection in the operating room, should also be taken into account.

The present invention thus provides cellular flaps from different cell types using the same procedure without any particular modifications. In addition, this method allows to obtain a large number of flaps quickly and without the need to use expensive cutluring plates.

The terms “expression vector” or “vector” refer to a nucleic acid that transduces, transforms, or infects a host cell, thereby causing the cell to produce nucleic acids and/or proteins other than those that are native to the cell, or to express nucleic acids and/or proteins in a manner that is not native to the cell.

The term “endogenous” refers to a molecule (e.g., a nucleic acid or a polypeptide) or process that occurs naturally, e.g., in a non-recombinant host cell.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the terms “operon” and “single transcription unit” are used interchangeably to refer to two or more contiguous coding regions (nucleotide sequences that encode a gene product such as an RNA or a protein) that are coordinately regulated by one or more controlling elements (e.g., a promoter). As used herein, the term “gene product” refers to RNA encoded by DNA (or vice versa) or protein that is encoded by an RNA or DNA, where a gene will typically comprise one or more nucleotide sequences that encode a protein, and may also include introns and other non-coding nucleotide sequences.

The term “heterologous nucleic acid” as used herein refers to a nucleic acid wherein at least one of the following is true: (a) the nucleic acid is foreign (“exogenous”) to (that is, not naturally found in) a given host cell; (b) the nucleic acid comprises a nucleotide sequence that is naturally found in (that is, is “endogenous to”) a given host cell, but the nucleotide sequence is produced in an unnatural (for example, greater than expected or greater than naturally found) amount in the cell; (c) the nucleic acid comprises a nucleotide sequence that differs in sequence from an endogenous nucleotide sequence, but the nucleotide sequence encodes the same protein (having the same or substantially the same amino acid sequence) and is produced in an unnatural (for example, greater than expected or greater than naturally found) amount in the cell; or (d) the nucleic acid comprises two or more nucleotide sequences that are not found in the same relationship to each other in nature (for example, the nucleic acid is recombinant).

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below).

Thus, e.g., the term “recombinant” polynucleotide or nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. The term “transformation” or “genetic modification” refers to a permanent or transient genetic change induced in a cell following introduction of new nucleic acid. Thus, a “genetically modified cell” is a host cell into which a new (e.g., exogenous; heterologous) nucleic acid has been introduced. Genetic change (“modification”) can be accomplished either by incorporation of the new DNA into the genome of the host cell, or by transient or stable maintenance of the new DNA as an episomal element. In eukaryotic cells, a permanent genetic change is generally achieved by introduction of the DNA into the genome of the cell. In prokaryotic cells, a permanent genetic change can be introduced into the chromosome or via extrachromosomal elements such as plasmids and expression vectors, which may contain one or more selectable markers to aid in their maintenance in the recombinant host cell. The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “operably linked” refers to a juxtaposition wherein the components so described are in a relationship permitting them to function in their intended manner. For instance, a promoter is operably linked to a nucleotide sequence if the promoter affects the transcription or expression of the nucleotide sequence.

A “host cell,” as used herein, denotes an in vitro eukaryotic cell (e.g., a yeast cell), which eukaryotic cell can be, or has been, used as a recipient for a nucleic acid, and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject eukaryotic host cell is a genetically modified eukaryotic host cell, by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A polynucleotide or polypeptide has a certain percent “sequence identity” to another polynucleotide orpolypeptide, meaning that, when aligned, that percentage ofbases or amino acids are the same, and in the same relative position, when comparing the two sequences.

In the present invention “at least 75% identity” means that the identity may be at least 75% or 80%, or 85% or 90% or 95% or 100% sequence identity to referred sequences. This applies to all the mentioned % of identity. Preferably, the % of identity relates to the full length of the referred sequence.

Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a protein” includes a plurality of such proteins, and so forth.

The term “functional variant” of a protein describes a protein that has a polypeptide sequence that is at least 70%, 75%, 80%, 85%, 90%, 95% or 99% identical to any one of the protein described herein. The “functional variant” protein may retain amino acids residues that are recognized as conserved for the protein, and may have non-conserved amino acid residues substituted or found to be of a different amino acid, or amino acid(s) inserted or deleted, but which does not affect or has insignificant effect its enzymatic activity as compared to the enzyme described herein. The “functional variant” protein has an activity that is identical or essentially identical to the activity of the protein described herein. The “functional variant” protein may be found in nature or be an engineered mutant thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

In the method above disclosed, thawed keratinocytes cells and feeder cells may be plated at the same time. Alternatively, it is possible to plate feeder cells and after 2 h-24 h thawing the transduced keratinocytes.

In the context of the present invention “IU” refers to “International Unit”.

In a preferred embodiment of the present invention, the genetically modified cells are cells that have been transduced with a retroviral vector carrying the cDNA of (or the nucleotide sequence encoding for) the beta-3 chain of laminin 5. However, results similar to those herein shown were obtained with similar products (e.g. retroviral vectors carrying different genes involved in EB). The retroviral vector may e.g. be an alpharetroviral vector, a gammaretroviral vector, a lentiviral vector or a spumaretroviral vector.

In the context of the present invention the “feeder cells” or “feeder” are cells preferably obtained according to the method disclosed in Rheinwald J G, Green H. 1975. Serial cultivation of strains of human epidermal keratinocytes: the formation of keratinizing colonies from single cells. Cell 6:331-343.

They correspond to a clone of murine cells isolated in the laboratory of. prof. Green H. (Rheinwald, J. et al 1975).

With the term “flap of cells” it is intended preferably a sheet of epithelial cells, comprising cells in a single layer or in multilayer able to recreate an epidermis ex vivo.

According to the present invention the fibrin substrate (or fibrin support) is preferably a fibrin gel which is obtainable by admixing fibrinogen and thrombin, thus obtaining a fibrinogen and thrombin composition or solution.

The step of detachment of the flap from the support in the method according to the present invention is preferably carried out by mechanical methods, e.g. using pliers or forceps. However, any method known by the skilled man may be used.

In the context of the present invention “similar to a sheet” is preferably intended as an intact cell sheet.

The term “comprises” when referred to the fibrin substrate can also be intended as “obtainable by admixing”.

The expression “genetically modified cells” includes cells comprising a heterologous nucleic acid, for example which were transduced or transfected with one or more nucleic acid.

Said heterologous nucleic acid is preferably a gene or a cDNA (or a nucleotide sequence encoding for a polypeptide) selected from the group consisting of: beta-3 chain of laminin 5, collagen 7, collagen 17 or combination thereof.

The starting cell may be a cell which expresses lower levels or doesn't express the heterologous nucleic acid as defined above. It can be transduced or transfected with a construct that will be integrated in the cell genome in place of the target endogenous gene or in different region, where said construct comprises a heterologous sequence of the gene of interest and in some cases also a selectable marker which allows to select the obtained genetically modified cells. Alternatively, the genetically modified cell may not comprise a sequence (also partial) of a particular nucleic acid encoding a specific protein or peptide, for example obtained by deletion of a genic sequence.

The washing solution used in the above method is preferably “Dulbecco's modified eagle medium (DMEM)”, supplemented with L-glutamine. The transport medium used in the above method is preferably “Dulbecco's modified eagle medium (DMEM)”, supplemented with L-glutamine. Preferably, the collagen VII is characterized by the sequence as disclosed in in the NCBI Data Bank with the Accession no.: NM_000094.3 (Col7A1). Its cDNA sequence is:

(SEQ ID NO: 1)
ATGACGCTGCGGCTTCTGGTGGCCGCGCTCTGCGCCGGGATCCTGGCAGA
GGCGCCCCGAGTGCGAGCCCAGCACAGGGAGAGAGTGACCTGCACGCGCC
TTTACGCCGCTGACATTGTGTTCTTACTGGATGGCTCCTCATCCATTGGC
CGCAGCAATTTCCGCGAGGTCCGCAGCTTTCTCGAAGGGCTGGTGCTGCC
TTTCTCTGGAGCAGCCAGTGCACAGGGTGTGCGCTTTGCCACAGTGCAGT
ACAGCGATGACCCACGGACAGAGTTCGGCCTGGATGCACTTGGCTCTGGG
GGTGATGTGATCCGCGCCATCCGTGAGCTTAGCTACAAGGGGGGCAACAC
TCGCACAGGGGCTGCAATTCTCCATGTGGCTGACCATGTCTTCCTGCCCC
AGCTGGCCCGACCTGGTGTCCCCAAGGTCTGCATCCTGATCACAGACGGG
AAGTCCCAGGACCTGGTGGACACAGCTGCCCAAAGGCTGAAGGGGCAGGG
GGTCAAGCTATTTGCTGTGGGGATCAAGAATGCTGACCCTGAGGAGCTGA
AGCGAGTTGCCTCACAGCCCACCAGTGACTTCTTCTTCTTCGTCAATGAC
TTCAGCATCTTGAGGACACTACTGCCCCTCGTTTCCCGGAGAGTGTGCAC
GACTGCTGGTGGCGTGCCTGTGACCCGACCTCCGGATGACTCGACCTCTG
CTCCACGAGACCTGGTGCTGTCTGAGCCAAGCAGCCAATCCTTGAGAGTA
CAGTGGACAGCGGCCAGTGGCCCTGTGACTGGCTACAAGGTCCAGTACAC
TCCTCTGACGGGGCTGGGACAGCCACTGCCGAGTGAGCGGCAGGAGGTGA
ACGTCCCAGCTGGTGAGACCAGTGTGCGGCTGCGGGGTCTCCGGCCACTG
ACCGAGTACCAAGTGACTGTGATTGCCCTCTACGCCAACAGCATCGGGGA
GGCTGTGAGCGGGACAGCTCGGACCACTGCCCTAGAAGGGCCGGAACTGA
CCATCCAGAATACCACAGCCCACAGCCTCCTGGTGGCCTGGCGGAGTGTG
CCAGGTGCCACTGGCTACCGTGTGACATGGCGGGTCCTCAGTGGTGGGCC
CACACAGCAGCAGGAGCTGGGCCCTGGGCAGGGTTCAGTGTTGCTGCGTG
ACTTGGAGCCTGGCACGGACTATGAGGTGACCGTGAGCACCCTATTTGGC
CGCAGTGTGGGGCCCGCCACTTCCCTGATGGCTCGCACTGACGCTTCTGT
TGAGCAGACCCTGCGCCCGGTCATCCTGGGCCCCACATCCATCCTCCTTT
CCTGGAACTTGGTGCCTGAGGCCCGTGGCTACCGGTTGGAATGGCGGCGT
GAGACTGGCTTGGAGCCACCGCAGAAGGTGGTACTGCCCTCTGATGTGAC
CCGCTACCAGTTGGATGGGCTGCAGCCGGGCACTGAGTACCGCCTCACAC
TCTACACTCTGCTGGAGGGCCACGAGGTGGCCACCCCTGCAACCGTGGTT
CCCACTGGACCAGAGCTGCCTGTGAGCCCTGTAACAGACCTGCAAGCCAC
CGAGCTGCCCGGGCAGCGGGTGCGAGTGTCCTGGAGCCCAGTCCCTGGTG
CCACCCAGTACCGCATCATTGTGCGCAGCACCCAGGGGGTTGAGCGGACC
CTGGTGCTTCCTGGGAGTCAGACAGCATTCGACTTGGATGACGTTCAGGC
TGGGCTTAGCTACACTGTGCGGGTGTCTGCTCGAGTGGGTCCCCGTGAGG
GCAGTGCCAGTGTCCTCACTGTCCGCCGGGAGCCGGAAACTCCACTTGCT
GTTCCAGGGCTGCGGGTTGTGGTGTCAGATGCAACGCGAGTGAGGGTGGC
CTGGGGACCCGTCCCTGGAGCCAGTGGATTTCGGATTAGCTGGAGCACAG
GCAGTGGTCCGGAGTCCAGCCAGACACTGCCCCCAGACTCTACTGCCACA
GACATCACAGGGCTGCAGCCTGGAACCACCTACCAGGTGGCTGTGTCGGT
ACTGCGAGGCAGAGAGGAGGGCCCTGCTGCAGTCATCGTGGCTCGAACGG
ACCCACTGGGCCCAGTGAGGACGGTCCATGTGACTCAGGCCAGCAGCTCA
TCTGTCACCATTACCTGGACCAGGGTTCCTGGCGCCACAGGATACAGGGT
TTCCTGGCACTCAGCCCACGGCCCAGAGAAATCCCAGTTGGTTTCTGGGG
AGGCCACGGTGGCTGAGCTGGATGGACTGGAGCCAGATACTGAGTATACG
GTGCATGTGAGGGCCCATGTGGCTGGCGTGGATGGGCCCCCTGCCTCTGT
GGTTGTGAGGACTGCCCCTGAGCCTGTGGGTCGTGTGTCGAGGCTGCAGA
TCCTCAATGCTTCCAGCGACGTTCTACGGATCACCTGGGTAGGGGTCACT
GGAGCCACAGCTTACAGACTGGCCTGGGGCCGGAGTGAAGGCGGCCCCAT
GAGGCACCAGATACTCCCAGGAAACACAGACTCTGCAGAGATCCGGGGTC
TCGAAGGTGGAGTCAGCTACTCAGTGCGAGTGACTGCACTTGTCGGGGAC
CGCGAGGGCACACCTGTCTCCATTGTTGTCACTACGCCGCCTGAGGCTCC
GCCAGCCCTGGGGACGCTTCACGTGGTGCAGCGCGGGGAGCACTCGCTGA
GGCTGCGCTGGGAGCCGGTGCCCAGAGCGCAGGGCTTCCTTCTGCACTGG
CAACCTGAGGGTGGCCAGGAACAGTCCCGGGTCCTGGGGCCCGAGCTCAG
CAGCTATCACCTGGACGGGCTGGAGCCAGCGACACAGTACCGCGTGAGGC
TGAGTGTCCTAGGGCCAGCTGGAGAAGGGCCCTCTGCAGAGGTGACTGCG
CGCACTGAGTCACCTCGTGTTCCAAGCATTGAACTACGTGTGGTGGACAC
CTCGATCGACTCGGTGACTTTGGCCTGGACTCCAGTGTCCAGGGCATCCA
GCTACATCCTATCCTGGCGGCCACTCAGAGGCCCTGGCCAGGAAGTGCCT
GGGTCCCCGCAGACACTTCCAGGGATCTCAAGCTCCCAGCGGGTGACAGG
GCTAGAGCCTGGCGTCTCTTACATCTTCTCCCTGACGCCTGTCCTGGATG
GTGTGCGGGGTCCTGAGGCATCTGTCACACAGACGCCAGTGTGCCCCCGT
GGCCTGGCGGATGTGGTGTTCCTACCACATGCCACTCAAGACAATGCTCA
CCGTGCGGAGGCTACGAGGAGGGTCCTGGAGCGTCTGGTGTTGGCACTTG
GGCCTCTTGGGCCACAGGCAGTTCAGGTTGGCCTGCTGTCTTACAGTCAT
CGGCCCTCCCCACTGTTCCCACTGAATGGCTCCCATGACCTTGGCATTAT
CTTGCAAAGGATCCGTGACATGCCCTACATGGACCCAAGTGGGAACAACC
TGGGCACAGCCGTGGTCACAGCTCACAGATACATGTTGGCACCAGATGCT
CCTGGGCGCCGCCAGCACGTACCAGGGGTGATGGTTCTGCTAGTGGATGA
ACCCTTGAGAGGTGACATATTCAGCCCCATCCGTGAGGCCCAGGCTTCTG
GGCTTAATGTGGTGATGTTGGGAATGGCTGGAGCGGACCCAGAGCAGCTG
CGTCGCTTGGCGCCGGGTATGGACTCTGTCCAGACCTTCTTCGCCGTGGA
TGATGGGCCAAGCCTGGACCAGGCAGTCAGTGGTCTGGCCACAGCCCTGT
GTCAGGCATCCTTCACTACTCAGCCCCGGCCAGAGCCCTGCCCAGTGTAT
TGTCCAAAGGGCCAGAAGGGGGAACCTGGAGAGATGGGCCTGAGAGGACA
AGTTGGGCCTCCTGGCGACCCTGGCCTCCCGGGCAGGACCGGTGCTCCCG
GCCCCCAGGGGCCCCCTGGAAGTGCCACTGCCAAGGGCGAGAGGGGCTTC
CCTGGAGCAGATGGGCGTCCAGGCAGCCCTGGCCGCGCCGGGAATCCTGG
GACCCCTGGAGCCCCTGGCCTAAAGGGCTCTCCAGGGTTGCCTGGCCCTC
GTGGGGACCCGGGAGAGCGAGGACCTCGAGGCCCAAAGGGGGAGCCGGGG
GCTCCCGGACAAGTCATCGGAGGTGAAGGACCTGGGCTTCCTGGGCGGAA
AGGGGACCCTGGACCATCGGGCCCCCCTGGACCTCGTGGACCACTGGGGG
ACCCAGGACCCCGTGGCCCCCCAGGGCTTCCTGGAACAGCCATGAAGGGT
GACAAAGGCGATCGTGGGGAGCGGGGTCCCCCTGGACCAGGTGAAGGTGG
CATTGCTCCTGGGGAGCCTGGGCTGCCGGGTCTTCCCGGAAGCCCTGGAC
CCCAAGGCCCCGTTGGCCCCCCTGGAAAGAAAGGAGAAAAAGGTGACTCT
GAGGATGGAGCTCCAGGCCTCCCAGGACAACCTGGGTCTCCGGGTGAGCA
GGGCCCACGGGGACCTCCTGGAGCTATTGGCCCCAAAGGTGACCGGGGCT
TTCCAGGGCCCCTGGGTGAGGCTGGAGAGAAGGGCGAACGTGGACCCCCA
GGCCCAGCGGGATCCCGGGGGCTGCCAGGGGTTGCTGGACGTCCTGGAGC
CAAGGGTCCTGAAGGGCCACCAGGACCCACTGGCCGCCAAGGAGAGAAGG
GGGAGCCTGGTCGCCCTGGGGACCCTGCAGTGGTGGGACCTGCTGTTGCT
GGACCCAAAGGAGAAAAGGGAGATGTGGGGCCCGCTGGGCCCAGAGGAGC
TACCGGAGTCCAAGGGGAACGGGGCCCACCCGGCTTGGTTCTTCCTGGAG
ACCCTGGCCCCAAGGGAGACCCTGGAGACCGGGGTCCCATTGGCCTTACT
GGCAGAGCAGGACCCCCAGGTGACTCAGGGCCTCCTGGAGAGAAGGGAGA
CCCTGGGCGGCCTGGCCCCCCAGGACCTGTTGGCCCCCGAGGACGAGATG
GTGAAGTTGGAGAGAAAGGTGACGAGGGTCCTCCGGGTGACCCGGGTTTG
CCTGGAAAAGCAGGCGAGCGTGGCCTTCGGGGGGCACCTGGAGTTCGGGG
GCCTGTGGGTGAAAAGGGAGACCAGGGAGATCCTGGAGAGGATGGACGAA
ATGGCAGCCCTGGATCATCTGGACCCAAGGGTGACCGTGGGGAGCCGGGT
CCCCCAGGACCCCCGGGACGGCTGGTAGACACAGGACCTGGAGCCAGAGA
GAAGGGAGAGCCTGGGGACCGCGGACAAGAGGGTCCTCGAGGGCCCAAGG
GTGATCCTGGCCTCCCTGGAGCCCCTGGGGAAAGGGGCATTGAAGGGTTT
CGGGGACCCCCAGGCCCACAGGGGGACCCAGGTGTCCGAGGCCCAGCAGG
AGAAAAGGGTGACCGGGGTCCCCCTGGGCTGGATGGCCGGAGCGGACTGG
ATGGGAAACCAGGAGCCGCTGGGCCCTCTGGGCCGAATGGTGCTGCAGGC
AAAGCTGGGGACCCAGGGAGAGACGGGCTTCCAGGCCTCCGTGGAGAACA
GGGCCTCCCTGGCCCCTCTGGTCCCCCTGGATTACCGGGAAAGCCAGGCG
AGGATGGCAAACCTGGCCTGAATGGAAAAAACGGAGAACCTGGGGACCCT
GGAGAAGACGGGAGGAAGGGAGAGAAAGGAGATTCAGGCGCCTCTGGGAG
AGAAGGTCGTGATGGCCCCAAGGGTGAGCGTGGAGCTCCTGGTATCCTTG
GACCCCAGGGGCCTCCAGGCCTCCCAGGGCCAGTGGGCCCTCCTGGCCAG
GGTTTTCCTGGTGTCCCAGGAGGCACGGGCCCCAAGGGTGACCGTGGGGA
GACTGGATCCAAAGGGGAGCAGGGCCTCCCTGGAGAGCGTGGCCTGCGAG
GAGAGCCTGGAAGTGTGCCGAATGTGGATCGGTTGCTGGAAACTGCTGGC
ATCAAGGCATCTGCCCTGCGGGAGATCGTGGAGACCTGGGATGAGAGCTC
TGGTAGCTTCCTGCCTGTGCCCGAACGGCGTCGAGGCCCCAAGGGGGACT
CAGGCGAACAGGGCCCCCCAGGCAAGGAGGGCCCCATCGGCTTTCCTGGA
GAACGCGGGCTGAAGGGCGACCGTGGAGACCCTGGCCCTCAGGGGCCACC
TGGTCTGGCCCTTGGGGAGAGGGGCCCCCCCGGGCCTTCCGGCCTTGCCG
GGGAGCCTGGAAAGCCTGGTATTCCCGGGCTCCCAGGCAGGGCTGGGGGT
GTGGGAGAGGCAGGAAGGCCAGGAGAGAGGGGAGAACGGGGAGAGAAAGG
AGAACGTGGAGAACAGGGCAGAGATGGCCCTCCTGGACTCCCTGGAACCC
CTGGGCCCCCCGGACCCCCTGGCCCCAAGGTGTCTGTGGATGAGCCAGGT
CCTGGACTCTCTGGAGAACAGGGACCCCCTGGACTCAAGGGTGCTAAGGG
GGAGCCGGGCAGCAATGGTGACCAAGGTCCCAAAGGAGACAGGGGTGTGC
CAGGCATCAAAGGAGACCGGGGAGAGCCTGGACCGAGGGGTCAGGACGGC
AACCCGGGTCTACCAGGAGAGCGTGGTATGGCTGGGCCTGAAGGGAAGCC
GGGTCTGCAGGGTCCAAGAGGCCCCCCTGGCCCAGTGGGTGGTCATGGAG
ACCCTGGACCACCTGGTGCCCCGGGTCTTGCTGGCCCTGCAGGACCCCAA
GGACCTTCTGGCCTGAAGGGGGAGCCTGGAGAGACAGGACCTCCAGGACG
GGGCCTGACTGGACCTACTGGAGCTGTGGGACTTCCTGGACCCCCCGGCC
CTTCAGGCCTTGTGGGTCCACAGGGGTCTCCAGGTTTGCCTGGACAAGTG
GGGGAGACAGGGAAGCCGGGAGCCCCAGGTCGAGATGGTGCCAGTGGAAA
AGATGGAGACAGAGGGAGCCCTGGTGTGCCAGGGTCACCAGGTCTGCCTG
GCCCTGTCGGACCTAAAGGAGAACCTGGCCCCACGGGGGCCCCTGGACAG
GCTGTGGTCGGGCTCCCTGGAGCAAAGGGAGAGAAGGGAGCCCCTGGAGG
CCTTGCTGGAGACCTGGTGGGTGAGCCGGGAGCCAAAGGTGACCGAGGAC
TGCCAGGGCCGCGAGGCGAGAAGGGTGAAGCTGGCCGTGCAGGGGAGCCC
GGAGACCCTGGGGAAGATGGTCAGAAAGGGGCTCCAGGACCCAAAGGTTT
CAAGGGTGACCCAGGAGTCGGGGTCCCGGGCTCCCCTGGGCCTCCTGGCC
CTCCAGGTGTGAAGGGAGATCTGGGCCTCCCTGGCCTGCCCGGTGCTCCT
GGTGTTGTTGGGTTCCCGGGTCAGACAGGCCCTCGAGGAGAGATGGGTCA
GCCAGGCCCTAGTGGAGAGCGGGGTCTGGCAGGCCCCCCAGGGAGAGAAG
GAATCCCAGGACCCCTGGGGCCACCTGGACCACCGGGGTCAGTGGGACCA
CCTGGGGCCTCTGGACTCAAAGGAGACAAGGGAGACCCTGGAGTAGGGCT
GCCTGGGCCCCGAGGCGAGCGTGGGGAGCCAGGCATCCGGGGTGAAGATG
GCCGCCCCGGCCAGGAGGGACCCCGAGGACTCACGGGGCCCCCTGGCAGC
AGGGGAGAGCGTGGGGAGAAGGGTGATGTTGGGAGTGCAGGACTAAAGGG
TGACAAGGGAGACTCAGCTGTGATCCTGGGGCCTCCAGGCCCACGGGGTG
CCAAGGGGGACATGGGTGAACGAGGGCCTCGGGGCTTGGATGGTGACAAA
GGACCTCGGGGAGACAATGGGGACCCTGGTGACAAGGGCAGCAAGGGAGA
GCCTGGTGACAAGGGCTCAGCCGGGTTGCCAGGACTGCGTGGACTCCTGG
GACCCCAGGGTCAACCTGGTGCAGCAGGGATCCCTGGTGACCCGGGATCC
CCAGGAAAGGATGGAGTGCCTGGTATCCGAGGAGAAAAAGGAGATGTTGG
CTTCATGGGTCCCCGGGGCCTCAAGGGTGAACGGGGAGTGAAGGGAGCCT
GTGGCCTTGATGGAGAGAAGGGAGACAAGGGAGAAGCTGGTCCCCCAGGC
CGCCCCGGGCTGGCAGGACACAAAGGAGAGATGGGGGAGCCTGGTGTGCC
GGGCCAGTCGGGGGCCCCTGGCAAGGAGGGCCTGATCGGTCCCAAGGGTG
ACCGAGGCTTTGACGGGCAGCCAGGCCCCAAGGGTGACCAGGGCGAGAAA
GGGGAGCGGGGAACCCCAGGAATTGGGGGCTTCCCAGGCCCCAGTGGAAA
TGATGGCTCTGCTGGTCCCCCAGGGCCACCTGGCAGTGTTGGTCCCAGAG
GCCCCGAAGGACTTCAGGGCCAGAAGGGTGAGCGAGGTCCCCCCGGAGAG
AGAGTGGTGGGGGCTCCTGGGGTCCCTGGAGCTCCTGGCGAGAGAGGGGA
GCAGGGGCGGCCAGGGCCTGCCGGTCCTCGAGGCGAGAAGGGAGAAGCTG
CACTGACGGAGGATGACATCCGGGGCTTTGTGCGCCAAGAGATGAGTCAG
CACTGTGCCTGCCAGGGCCAGTTCATCGCATCTGGATCACGACCCCTCCC
TAGTTATGCTGCAGACACTGCCGGCTCCCAGCTCCATGCTGTGCCTGTGC
TCCGCGTCTCTCATGCAGAGGAGGAAGAGCGGGTACCCCCTGAGGATGAT
GAGTACTCTGAATACTCCGAGTATTCTGTGGAGGAGTACCAGGACCCTGA
AGCTCCTTGGGATAGTGATGACCCCTGTTCCCTGCCACTGGATGAGGGCT
CCTGCACTGCCTACACCCTGCGCTGGTACCATCGGGCTGTGACAGGCAGC
ACAGAGGCCTGTCACCCTTTTGTCTATGGTGGCTGTGGAGGGAATGCCAA
CCGTTTTGGGACCCGTGAGGCCTGCGAGCGCCGCTGCCCACCCCGGGTGG
TCCAGAGCCAGGGGACAGGTACTGCCCAGGACTGA

Its protein sequence is:

(SEQ ID NO: 2)
MTLRLLVAALCAGILAEAPRVRAQHRERVTCTRLYAADIVFLLDGSSSIG
RSNFREVRSFLEGLVLPFSGAASAQGVRFATVQYSDDPRTEFGLDALGSG
GDVIRAIRELSYKGGNTRTGAAILHVADHVFLPQLARPGVPKVCILITDG
KSQDLVDTAAQRLKGQGVKLFAVGIKNADPEELKRVASQPTSDFFFFVND
FSILRTLLPLVSRRVCTTAGGVPVTRPPDDSTSAPRDLVLSEPSSQSLRV
QWTAASGPVTGYKVQYTPLTGLGQPLPSERQEVNVPAGETSVRLRGLRPL
TEYQVTVIALYANSIGEAVSGTARTTALEGPELTIQNTTAHSLLVAWRSV
PGATGYRVTWRVLSGGPTQQQELGPGQGSVLLRDLEPGTDYEVTVSTLFG
RSVGPATSLMARTDASVEQTLRPVILGPTSILLSWNLVPEARGYRLEWRR
ETGLEPPQKVVLPSDVTRYQLDGLQPGTEYRLTLYTLLEGHEVATPATVV
PTGPELPVSPVTDLQATELPGQRVRVSWSPVPGATQYRIIVRSTQGVERT
LVLPGSQTAFDLDDVQAGLSYTVRVSARVGPREGSASVLTVRREPETPLA
VPGLRVVVSDATRVRVAWGPVPGASGFRISWSTGSGPESSQTLPPDSTAT
DITGLQPGTTYQVAVSVLRGREEGPAAVIVARTDPLGPVRTVHVTQASSS
SVTITWTRVPGATGYRVSWHSAHGPEKSQLVSGEATVAELDGLEPDTEYT
VHVRAHVAGVDGPPASVVVRTAPEPVGRVSRLQILNASSDVLRITWVGVT
GATAYRLAWGRSEGGPMRHQILPGNTDSAEIRGLEGGVSYSVRVTALVGD
REGTPVSIVVTTPPEAPPALGTLHVVQRGEHSLRLRWEPVPRAQGFLLHW
QPEGGQEQSRVLGPELSSYHLDGLEPATQYRVRLSVLGPAGEGPSAEVTA
RTESPRVPSIELRVVDTSIDSVTLAWTPVSRASSYILSWRPLRGPGQEVP
GSPQTLPGISSSQRVTGLEPGVSYIFSLTPVLDGVRGPEASVTQTPVCPR
GLADVVFLPHATQDNAHRAEATRRVLERLVLALGPLGPQAVQVGLLSYSH
RPSPLFPLNGSHDLGIILQRIRDMPYMDPSGNNLGTAVVTAHRYMLAPDA
PGRRQHVPGVMVLLVDEPLRGDIFSPIREAQASGLNVVMLGMAGADPEQL
RRLAPGMDSVQTFFAVDDGPSLDQAVSGLATALCQASFTTQPRPEPCPVY
CPKGQKGEPGEMGLRGQVGPPGDPGLPGRTGAPGPQGPPGSATAKGERGF
PGADGRPGSPGRAGNPGTPGAPGLKGSPGLPGPRGDPGERGPRGPKGEPG
APGQVIGGEGPGLPGRKGDPGPSGPPGPRGPLGDPGPRGPPGLPGTAMKG
DKGDRGERGPPGPGEGGIAPGEPGLPGLPGSPGPQGPVGPPGKKGEKGDS
EDGAPGLPGQPGSPGEQGPRGPPGAIGPKGDRGFPGPLGEAGEKGERGPP
GPAGSRGLPGVAGRPGAKGPEGPPGPTGRQGEKGEPGRPGDPAVVGPAVA
GPKGEKGDVGPAGPRGATGVQGERGPPGLVLPGDPGPKGDPGDRGPIGLT
GRAGPPGDSGPPGEKGDPGRPGPPGPVGPRGRDGEVGEKGDEGPPGDPGL
PGKAGERGLRGAPGVRGPVGEKGDQGDPGEDGRNGSPGSSGPKGDRGEPG
PPGPPGRLVDTGPGAREKGEPGDRGQEGPRGPKGDPGLPGAPGERGIEGF
RGPPGPQGDPGVRGPAGEKGDRGPPGLDGRSGLDGKPGAAGPSGPNGAAG
KAGDPGRDGLPGLRGEQGLPGPSGPPGLPGKPGEDGKPGLNGKNGEPGDP
GEDGRKGEKGDSGASGREGRDGPKGERGAPGILGPQGPPGLPGPVGPPGQ
GFPGVPGGTGPKGDRGETGSKGEQGLPGERGLRGEPGSVPNVDRLLETAG
IKASALREIVETWDESSGSFLPVPERRRGPKGDSGEQGPPGKEGPIGFPG
ERGLKGDRGDPGPQGPPGLALGERGPPGPSGLAGEPGKPGIPGLPGRAGG
VGEAGRPGERGERGEKGERGEQGRDGPPGLPGTPGPPGPPGPKVSVDEPG
PGLSGEQGPPGLKGAKGEPGSNGDQGPKGDRGVPGIKGDRGEPGPRGQDG
NPGLPGERGMAGPEGKPGLQGPRGPPGPVGGHGDPGPPGAPGLAGPAGPQ
GPSGLKGEPGETGPPGRGLTGPTGAVGLPGPPGPSGLVGPQGSPGLPGQV
GETGKPGAPGRDGASGKDGDRGSPGVPGSPGLPGPVGPKGEPGPTGAPGQ
AVVGLPGAKGEKGAPGGLAGDLVGEPGAKGDRGLPGPRGEKGEAGRAGEP
GDPGEDGQKGAPGPKGFKGDPGVGVPGSPGPPGPPGVKGDLGLPGLPGAP
GVVGFPGQTGPRGEMGQPGPSGERGLAGPPGREGIPGPLGPPGPPGSVGP
PGASGLKGDKGDPGVGLPGPRGERGEPGIRGEDGRPGQEGPRGLTGPPGS
RGERGEKGDVGSAGLKGDKGDSAVILGPPGPRGAKGDMGERGPRGLDGDK
GPRGDNGDPGDKGSKGEPGDKGSAGLPGLRGLLGPQGQPGAAGIPGDPGS
PGKDGVPGIRGEKGDVGFMGPRGLKGERGVKGACGLDGEKGDKGEAGPPG
RPGLAGHKGEMGEPGVPGQSGAPGKEGLIGPKGDRGFDGQPGPKGDQGEK
GERGTPGIGGFPGPSGNDGSAGPPGPPGSVGPRGPEGLQGQKGERGPPGE
RVVGAPGVPGAPGERGEQGRPGPAGPRGEKGEAALTEDDIRGFVRQEMSQ
HCACQGQFIASGSRPLPSYAADTAGSQLHAVPVLRVSHAEEEERVPPEDD
EYSEYSEYSVEEYQDPEAPWDSDDPCSLPLDEGSCTAYTLRWYHRAVTGS
TEACHPFVYGGCGGNANRFGTREACERRCPPRVVQSQGTGTAQD

Preferably, collagen XVII is characterized by the sequence disclosed in the NCBI Data Bank with the Accession no: NM_000494.3 (COL17A1). Its cDNA sequence is:

(SEQ ID NO: 3)
ATGGATGTAACCAAGAAAAACAAACGAGATGGAACTGAAGTCACTGAGAG
AATTGTCACTGAAACAGTAACCACAAGACTTACATCCTTACCACCAAAAG
GCGGGACCAGCAATGGCTATGCTAAAACAGCCTCTCTTGGTGGAGGGAGC
CGGCTGGAGAAACAAAGCCTGACTCATGGCAGCAGCGGCTACATAAACTC
AACTGGAAGCACACGAGGCCATGCCTCCACCTCTAGTTACAGGAGGGCTC
ACTCACCTGCCTCCACTCTGCCCAACTCCCCAGGCTCAACCTTTGAAAGG
AAAACTCACGTTACCCGCCATGCGTATGAAGGGAGCTCCAGTGGCAACTC
TTCTCCGGAGTACCCTCGGAAGGAATTTGCATCTTCTTCAACCAGAGGAC
GGAGTCAAACACGAGAGAGTGAAATTCGAGTTCGACTGCAGAGTGCGTCC
CCATCCACCCGATGGACAGAATTGGATGATGTTAAGCGTTTGCTCAAGGG
GAGTCGATCGGCAAGTGTGAGCCCCACCCGGAATTCCTCCAACACACTCC
CCATCCCCAAGAAAGGCACTGTGGAGACCAAAATTGTGACAGCGAGCTCC
CAGTCGGTGTCAGGCACCTACGATGCAACGATCCTGGATGCCAACCTTCC
CTCCCATGTGTGGTCCTCCACCCTGCCCGCGGGGTCCTCCATGGGGACCT
ATCACAACAACATGACAACCCAGAGCTCATCCCTCCTCAACACCAATGCC
TACTCTGCGGGATCAGTCTTCGGAGTTCCAAACAACATGGCGTCCTGCTC
ACCCACTTTGCACCCTGGACTCAGCACATCCTCCTCAGTGTTTGGCATGC
AGAACAATCTGGCCCCCAGCTTGACCACCCTGTCCCATGGCACCACCACC
ACTTCCACAGCATATGGGGTGAAGAAAAACATGCCCCAGAGTCCTGCGGC
TGTGAACACTGGCGTTTCCACCTCCGCCGCCTGCACCACAAGTGTGCAGA
GCGATGACCTTTTGCACAAGGACTGCAAGTTCCTGATCCTAGAGAAAGAC
AACACACCTGCCAAGAAGGAGATGGAGCTGCTCATCATGACCAAGGACAG
CGGGAAGGTCTTTACAGCCTCCCCTGCCAGCATCGCTGCAACTTCTTTTT
CAGAAGACACCCTAAAAAAAGAAAAGCAAGCTGCCTACAATGCTGACTCA
GGCCTAAAAGCCGAAGCTAATGGAGACCTGAAGACTGTGTCCACAAAGGG
CAAGACCACCACTGCAGATATCCACAGCTACGGCAGCAGTGGTGGTGGTG
GCAGTGGAGGAGGTGGCGGTGTTGGTGGCGCTGGCGGCGGCCCTTGGGGA
CCAGCGCCAGCCTGGTGCCCCTGCGGCTCCTGCTGCAGCTGGTGGAAGTG
GCTGCTGGGCCTGCTGCTCACCTGGCTGCTACTCCTGGGGCTGCTCTTCG
GCCTCATTGCTCTGGCGGAGGAGGTGAGGAAGCTGAAGGCGCGTGTGGAT
GAGCTGGAGAGGATCAGGAGGAGCATACTGCCCTATGGGGACAGCATGGA
TAGAATAGAAAAGGACCGCCTCCAGGGCATGGCACCCGCGGCGGGAGCAG
ACCTGGACAAAATTGGGCTGCACAGTGACAGCCAGGAGGAGCTCTGGATG
TTCGTGAGGAAGAAGCTAATGATGGAACAGGAAAATGGAAATCTCCGAGG
AAGCCCTGGCCCTAAAGGTGACATGGGAAGTCCAGGCCCTAAAGGAGATC
GAGGGTTCCCTGGGACTCCAGGTATCCCTGGGCCCTTGGGCCACCCAGGT
CCACAAGGACCAAAGGGTCAAAAAGGCAGCGTGGGAGATCCTGGCATGGA
AGGCCCCATGGGCCAGAGAGGGCGAGAAGGCCCCATGGGACCTCGTGGTG
AGGCAGGGCCTCCTGGATCTGGAGAGAAAGGGGAAAGAGGGGCTGCTGGT
GAACCAGGTCCTCATGGCCCACCTGGTGTCCCAGGTTCTGTGGGTCCCAA
AGGTTCCAGCGGCTCTCCTGGCCCACAGGGCCCTCCAGGTCCTGTAGGTC
TCCAAGGGCTCCGAGGTGAAGTAGGACTTCCTGGTGTCAAAGGTGACAAA
GGACCAATGGGACCACCAGGACCCAAAGGTGACCAGGGTGAGAAAGGACC
TCGAGGCCTCACAGGCGAGCCTGGCATGAGAGGTTTGCCTGGTGCTGTTG
GTGAGCCCGGGGCTAAAGGAGCAATGGGTCCTGCTGGCCCAGACGGACAC
CAAGGCCCAAGAGGTGAACAAGGTCTTACTGGGATGCCTGGAATCCGTGG
CCCACCAGGACCTTCTGGAGACCCAGGAAAGCCAGGTCTCACAGGACCCC
AGGGACCTCAGGGACTTCCCGGTACCCCTGGCCGACCAGGAATAAAAGGT
GAACCAGGAGCTCCAGGCAAGATCGTGACTTCGGAGGGGTCATCGATGCT
CACTGTCCCAGGCCCCCCAGGACCTCCTGGAGCCATGGGACCCCCAGGAC
CTCCAGGTGCCCCAGGCCCTGCCGGCCCAGCTGGTCTCCCAGGACATCAA
GAAGTTCTTAATTTACAAGGTCCCCCAGGCCCACCCGGCCCACGCGGGCC
ACCAGGGCCTTCCATTCCAGGCCCACCAGGACCCCGAGGCCCACCAGGGG
AGGGTTTGCCAGGCCCACCAGGCCCACCAGGATCGTTCCTGTCCAACTCA
GAAACCTTCCTCTCCGGCCCCCCAGGCCCACCTGGCCCCCCAGGTCCCAA
GGGAGACCAAGGTCCCCCAGGCCCCAGAGGACACCAAGGCGAGCAAGGCC
TCCCAGGTTTCTCAACCTCAGGGTCCAGTTCTTTCGGACTCAACCTTCAG
GGACCACCAGGCCCACCTGGCCCCCAGGGACCCAAAGGTGACAAAGGTGA
TCCAGGTGTTCCAGGGGCTCTTGGCATTCCTAGTGGTCCTTCTGAAGGGG
GATCATCAAGTACCATGTACGTGTCAGGCCCGCCAGGGCCCCCTGGGCCC
CCTGGGCCTCCGGGCTCTATCAGCAGCTCTGGCCAGGAGATTCAGCAGTA
CATCTCTGAGTACATGCAGAGTGACAGTATTAGATCTTACCTATCCGGAG
TTCAGGGTCCCCCAGGCCCACCTGGTCCCCCAGGACCTGTCACCACCATC
ACAGGCGAGACTTTCGACTACTCAGAGCTGGCAAGCCACGTTGTGAGCTA
CTTACGGACTTCGGGGTACGGTGTCAGCTTGTTCTCGTCCTCCATCTCTT
CTGAAGACATTCTGGCTGTGCTGCAGCGGGATGACGTGCGTCAGTACCTA
CGTCAGTACTTGATGGGCCCTCGGGGTCCGCCAGGGCCACCAGGAGCCAG
TGGAGATGGGTCCCTCCTGTCTTTGGACTATGCAGAGCTGAGTAGTCGCA
TTCTCAGCTACATGTCGAGTTCTGGGATCAGCATTGGGCTTCCTGGTCCC
CCGGGGCCCCCTGGCTTGCCGGGAACCTCCTATGAGGAGCTCCTCTCCTT
GCTGCGAGGGTCTGAATTCAGAGGCATCGTTGGACCCCCAGGTCCCCCGG
GTCCACCAGGGATCCCAGGCAATGTGTGGTCCAGCATCAGCGTGGAGGAC
CTCTCGTCTTACTTACATACTGCCGGCTTGTCATTCATCCCAGGCCCTCC
AGGACCTCCTGGTCCCCCAGGGCCTCGAGGGCCCCCGGGTGTCTCAGGAG
CCCTGGCAACCTATGCAGCTGAAAACAGCGACAGCTTCCGGAGCGAGCTG
ATCAGCTACCTCACAAGTCCTGATGTGCGCAGCTTCATTGTTGGCCCCCC
AGGCCCTCCTGGGCCGCAGGGACCCCCTGGGGACAGCCGCCTCCTGTCCA
CGGATGCCTCCCACAGTCGGGGTAGCAGCTCCTCCTCACACAGCTCATCT
GTCAGGCGGGGCAGCTCCTACAGCTCTTCCATGAGCACAGGAGGAGGTGG
TGCAGGCTCCCTGGGTGCAGGCGGTGCCTTTGGTGAAGCTGCAGGAGACA
GGGGTCCCTATGGCACTGACATCGGCCCAGGCGGAGGCTATGGGGCAGCA
GCAGAAGGCGGCATGTATGCTGGCAATGGCGGACTATTGGGAGCTGACTT
TGCTGGAGATCTGGATTACAATGAGCTGGCTGTGAGGGTGTCAGAGAGCA
TGCAGCGTCAGGGCCTACTGCAAGGGATGGCCTACACTGTCCAGGGCCCA
CCAGGCCAGCCTGGGCCACAGGGGCCACCCGGCATCAGCAAGGTCTTCTC
TGCCTACAGCAACGTGACTGCGGACCTCATGGACTTCTTCCAAACTTATG
GAGCCATTCAAGGACCCCCTGGGCAAAAAGGAGAGATGGGCACTCCAGGA
CCCAAAGGTGACAGGGGCCCTGCTGGGCCACCAGGTCATCCTGGGCCACC
TGGCCCTCGAGGACACAAGGGAGAAAAAGGAGACAAAGGTGACCAAGTCT
ATGCTGGGCGGAGAAGGAGAAGAAGTATTGCTGTCAAGCCGTGA

Its protein sequence is:

(SEQ ID NO: 4)
MDVTKKNKRDGTEVTERIVTETVTTRLTSLPPKGGTSNGYAKTASLGGGS
RLEKQSLTHGSSGYINSTGSTRGHASTSSYRRAHSPASTLPNSPGSTFER
KTHVTRHAYEGSSSGNSSPEYPRKEFASSSTRGRSQTRESEIRVRLQSAS
PSTRWTELDDVKRLLKGSRSASVSPTRNSSNTLPIPKKGTVETKIVTASS
QSVSGTYDATILDANLPSHVWSSTLPAGSSMGTYHNNMTTQSSSLLNTNA
YSAGSVFGVPNNMASCSPTLHPGLSTSSSVFGMQNNLAPSLTTLSHGTTT
TSTAYGVKKNMPQSPAAVNTGVSTSAACTTSVQSDDLLHKDCKFLILEKD
NTPAKKEMELLIMTKDSGKVFTASPASIAATSFSEDTLKKEKQAAYNADS
GLKAEANGDLKTVSTKGKTTTADIHSYGSSGGGGSGGGGGVGGAGGGPWG
PAPAWCPCGSCCSWWKWLLGLLLTWLLLLGLLFGLIALAEEVRKLKARVD
ELERIRRSILPYGDSMDRIEKDRLQGMAPAAGADLDKIGLHSDSQEELWM
FVRKKLMMEQENGNLRGSPGPKGDMGSPGPKGDRGFPGTPGIPGPLGHPG
PQGPKGQKGSVGDPGMEGPMGQRGREGPMGPRGEAGPPGSGEKGERGAAG
EPGPHGPPGVPGSVGPKGSSGSPGPQGPPGPVGLQGLRGEVGLPGVKGDK
GPMGPPGPKGDQGEKGPRGLTGEPGMRGLPGAVGEPGAKGAMGPAGPDGH
QGPRGEQGLTGMPGIRGPPGPSGDPGKPGLTGPQGPQGLPGTPGRPGIKG
EPGAPGKIVTSEGSSMLTVPGPPGPPGAMGPPGPPGAPGPAGPAGLPGHQ
EVLNLQGPPGPPGPRGPPGPSIPGPPGPRGPPGEGLPGPPGPPGSFLSNS
ETFLSGPPGPPGPPGPKGDQGPPGPRGHQGEQGLPGFSTSGSSSFGLNLQ
GPPGPPGPQGPKGDKGDPGVPGALGIPSGPSEGGSSSTMYVSGPPGPPGP
PGPPGSISSSGQEIQQYISEYMQSDSIRSYLSGVQGPPGPPGPPGPVTTI
TGETFDYSELASHVVSYLRTSGYGVSLFSSSISSEDILAVLQRDDVRQYL
RQYLMGPRGPPGPPGASGDGSLLSLDYAELSSRILSYMSSSGISIGLPGP
PGPPGLPGTSYEELLSLLRGSEFRGIVGPPGPPGPPGIPGNVWSSISVED
LSSYLHTAGLSFIPGPPGPPGPPGPRGPPGVSGALATYAAENSDSFRSEL
ISYLTSPDVRSFIVGPPGPPGPQGPPGDSRLLSTDASHSRGSSSSSHSSS
VRRGSSYSSSMSTGGGGAGSLGAGGAFGEAAGDRGPYGTDIGPGGGYGAA
AEGGMYAGNGGLLGADFAGDLDYNELAVRVSESMQRQGLLQGMAYTVQGP
PGQPGPQGPPGISKVFSAYSNVTADLMDFFQTYGAIQGPPGQKGEMGTPG
PKGDRGPAGPPGHPGPPGPRGHKGEKGDKGDQVYAGRRRRRSIAVKP

Preferably, the beta-3 chain of laminin 5 is characterized by the sequence disclosed in the NCBI Data Bank with the Accession no.:NM_000228-Q13751 (LAMB3). Its cDNA sequence is:

(SEQ ID NO: 5)
ATGAGACCATTCTTCCTCTTGTGTTTTGCCCTGCCTGGCCTCCTGCATGC
CCAACAAGCCTGCTCCCGTGGGGCCTGCTATCCACCTGTTGGGGACCTGC
TTGTTGGGAGGACCCGGTTTCTCCGAGCTTCATCTACCTGTGGACTGACC
AAGCCTGAGACCTACTGCACCCAGTATGGCGAGTGGCAGATGAAATGCTG
CAAGTGTGACTCCAGGCAGCCTCACAACTACTACAGTCACCGAGTAGAGA
ATGTGGCTTCATCCTCCGGCCCCATGCGCTGGTGGCAGTCACAGAATGAT
GTGAACCCTGTCTCTCTGCAGCTGGACCTGGACAGGAGATTCCAGCTTCA
AGAAGTCATGATGGAGTTCCAGGGGCCCATGCCCGCCGGCATGCTGATTG
AGCGCTCCTCAGACTTCGGTAAGACCTGGCGAGTGTACCAGTACCTGGCT
GCCGACTGCACCTCCACCTTCCCTCGGGTCCGCCAGGGTCGGCCTCAGAG
CTGGCAGGATGTTCGGTGCCAGTCCCTGCCTCAGAGGCCTAATGCACGCC
TAAATGGGGGGAAGGTCCAACTTAACCTTATGGATTTAGTGTCTGGGATT
CCAGCAACTCAAAGTCAAAAAATTCAAGAGGTGGGGGAGATCACAAACTT
GAGAGTCAATTTCACCAGGCTGGCCCCTGTGCCCCAAAGGGGCTACCACC
CTCCCAGCGCCTACTATGCTGTGTCCCAGCTCCGTCTGCAGGGGAGCTGC
TTCTGTCACGGCCATGCTGATCGCTGCGCACCCAAGCCTGGGGCCTCTGC
AGGCCCCTCCACCGCTGTGCAGGTCCACGATGTCTGTGTCTGCCAGCACA
ACACTGCCGGCCCAAATTGTGAGCGCTGTGCACCCTTCTACAACAACCGG
CCCTGGAGACCGGCGGAGGGCCAGGACGCCCATGAATGCCAAAGGTGCGA
CTGCAATGGGCACTCAGAGACATGTCACTTTGACCCCGCTGTGTTTGCCG
CCAGCCAGGGGGCATATGGAGGTGTGTGTGACAATTGCCGGGACCACACC
GAAGGCAAGAACTGTGAGCGGTGTCAGCTGCACTATTTCCGGAACCGGCG
CCCGGGAGCTTCCATTCAGGAGACCTGCATCTCCTGCGAGTGTGATCCGG
ATGGGGCAGTGCCAGGGGCTCCCTGTGACCCAGTGACCGGGCAGTGTGTG
TGCAAGGAGCATGTGCAGGGAGAGCGCTGTGACCTATGCAAGCCGGGCTT
CACTGGACTCACCTACGCCAACCCGCAGGGCTGCCACCGCTGTGACTGCA
ACATCCTGGGGTCCCGGAGGGACATGCCGTGTGACGAGGAGAGTGGGCGC
TGCCTTTGTCTGCCCAACGTGGTGGGTCCCAAATGTGACCAGTGTGCTCC
CTACCACTGGAAGCTGGCCAGTGGCCAGGGCTGTGAACCGTGTGCCTGCG
ACCCGCACAACTCCCTCAGCCCACAGTGCAACCAGTTCACAGGGCAGTGC
CCCTGTCGGGAAGGCTTTGGTGGCCTGATGTGCAGCGCTGCAGCCATCCG
CCAGTGTCCAGACCGGACCTATGGAGACGTGGCCACAGGATGCCGAGCCT
GTGACTGTGATTTCCGGGGAACAGAGGGCCCGGGCTGCGACAAGGCATCA
GGCCGCTGCCTCTGCCGCCCTGGCTTGACCGGGCCCCGCTGTGACCAGTG
CCAGCGAGGCTACTGTAATCGCTACCCGGTGTGCGTGGCCTGCCACCCTT
GCTTCCAGACCTATGATGCGGACCTCCGGGAGCAGGCCCTGCGCTTTGGT
AGACTCCGCAATGCCACCGCCAGCCTGTGGTCAGGGCCTGGGCTGGAGGA
CCGTGGCCTGGCCTCCCGGATCCTAGATGCAAAGAGTAAGATTGAGCAGA
TCCGAGCAGTTCTCAGCAGCCCCGCAGTCACAGAGCAGGAGGTGGCTCAG
GTGGCCAGTGCCATCCTCTCCCTCAGGCGAACTCTCCAGGGCCTGCAGCT
GGATCTGCCCCTGGAGGAGGAGACGTTGTCCCTTCCGAGAGACCTGGAGA
GTCTTGACAGAAGCTTCAATGGTCTCCTTACTATGTATCAGAGGAAGAGG
GAGCAGTTTGAAAAAATAAGCAGTGCTGATCCTTCAGGAGCCTTCCGGAT
GCTGAGCACAGCCTACGAGCAGTCAGCCCAGGCTGCTCAGCAGGTCTCCG
ACAGCTCGCGCCTTTTGGACCAGCTCAGGGACAGCCGGAGAGAGGCAGAG
AGGCTGGTGCGGCAGGCGGGAGGAGGAGGAGGCACCGGCAGCCCCAAGCT
TGTGGCCCTGAGGCTGGAGATGTCTTCGTTGCCTGACCTGACACCCACCT
TCAACAAGCTCTGTGGCAACTCCAGGCAGATGGCTTGCACCCCAATATCA
TGCCCTGGTGAGCTATGTCCCCAAGACAATGGCACAGCCTGTGGCTCCCG
CTGCAGGGGTGTCCTTCCCAGGGCCGGTGGGGCCTTCTTGATGGCGGGGC
AGGTGGCTGAGCAGCTGCGGGGCTTCAATGCCCAGCTCCAGCGGACCAGG
CAGATGATTAGGGCAGCCGAGGAATCTGCCTCACAGATTCAATCCAGTGC
CCAGCGCTTGGAGACCCAGGTGAGCGCCAGCCGCTCCCAGATGGAGGAAG
ATGTCAGACGCACACGGCTCCTAATCCAGCAGGTCCGGGACTTCCTAACA
GACCCCGACACTGATGCAGCCACTATCCAGGAGGTCAGCGAGGCCGTGCT
GGCCCTGTGGCTGCCCACAGACTCAGCTACTGTTCTGCAGAAGATGAATG
AGATCCAGGCCATTGCAGCCAGGCTCCCCAACGTGGACTTGGTGCTGTCC
CAGACCAAGCAGGACATTGCGCGTGCCCGCCGGTTGCAGGCTGAGGCTGA
GGAAGCCAGGAGCCGAGCCCATGCAGTGGAGGGCCAGGTGGAAGATGTGG
TTGGGAACCTGCGGCAGGGGACAGTGGCACTGCAGGAAGCTCAGGACACC
ATGCAAGGCACCAGCCGCTCCCTTCGGCTTATCCAGGACAGGGTTGCTGA
GGTTCAGCAGGTACTGCGGCCAGCAGAAAAGCTGGTGACAAGCATGACCA
AGCAGCTGGGTGACTTCTGGACACGGATGGAGGAGCTCCGCCACCAAGCC
CGGCAGCAGGGGGCAGAGGCAGTCCAGGCCCAGCAGCTTGCGGAAGGTGC
CAGCGAGCAGGCATTGAGTGCCCAAGAGGGATTTGAGAGAATAAAACAAA
AGTATGCTGAGTTGAAGGACCGGTTGGGTCAGAGTTCCATGCTGGGTGAG
CAGGGTGCCCGGATCCAGAGTGTGAAGACAGAGGCAGAGGAGCTGTTTGG
GGAGACCATGGAGATGATGGACAGGATGAAAGACATGGAGTTGGAGCTGC
TGCGGGGCAGCCAGGCCATCATGCTGCGCTCAGCGGACCTGACAGGACTG
GAGAAGCGTGTGGAGCAGATCCGTGACCACATCAATGGGCGCGTGCTCTA
CTATGCCACCTGCAAGTGA

Its protein sequence is:

(SEQ ID NO: 6)
MRPFFLLCFALPGLLHAQQACSRGACYPPVGDLLVGRTRFLRASSTCGLT
KPETYCTQYGEWQMKCCKCDSRQPHNYYSHRVENVASSSGPMRWWQSQND
VNPVSLQLDLDRRFQLQEVMMEFQGPMPAGMLIERSSDFGKTWRVYQYLA
ADCTSTFPRVRQGRPQSWQDVRCQSLPQRPNARLNGGKVQLNLMDLVSGI
PATQSQKIQEVGEITNLRVNFTRLAPVPQRGYHPPSAYYAVSQLRLQGSC
FCHGHADRCAPKPGASAGPSTAVQVHDVCVCQHNTAGPNCERCAPFYNNR
PWRPAEGQDAHECQRCDCNGHSETCHFDPAVFAASQGAYGGVCDNCRDHT
EGKNCERCQLHYFRNRRPGASIQETCISCECDPDGAVPGAPCDPVTGQCV
CKEHVQGERCDLCKPGFTGLTYANPQGCHRCDCNILGSRRDMPCDEESGR
CLCLPNVVGPKCDQCAPYHWKLASGQGCEPCACDPHNSLSPQCNQFTGQC
PCREGEGGLMCSAAAIRQCPDRTYGDVATGCRACDCDFRGTEGPGCDKAS
GRCLCRPGLTGPRCDQCQRGYCNRYPVCVACHPCFQTYDADLREQALRFG
RLRNATASLWSGPGLEDRGLASRILDAKSKIEQTRAVLSSPAVTEQEVAQ
VASAILSLRRTLQGLQLDLPLEEETLSLPRDLESLDRSFNGLLTMYQRKR
EQFEKISSADPSGAFRMLSTAYEQSAQAAQQVSDSSRLLDQLRDSRREAE
RLVRQAGGGGGTGSPKLVALRLEMSSLPDLTPTENKLCGNSRQMACTPIS
CPGELCPQDNGTACGSRCRGVLPRAGGAFLMAGQVAEQLRGENAQLQRTR
QMIRAAEESASQIQSSAQRLETQVSASRSQMEEDVRRTRLLIQQVRDFLT
DPDTDAATIQEVSEAVLALWLPTDSATVLQKMNEIQATAARLPNVDLVLS
QTKQDIARARRLQAEAEEARSRAHAVEGQVEDVVGNLRQGTVALQEAQDT
MQGTSRSLRLIQDRVAEVQQVLRPAEKLVTSMTKQLGDFWTRMEELRHQA
RQQGAEAVQAQQLAEGASEQALSAQEGFERIKQKYAELKDRLGQSSMLGE
QGARIQSVKTEAEELFGETMEMMDRMKDMELELLRGSQAIMLRSADLTGL
EKRVEQIRDHINGRVLYYATCK

Preferably, the LAMA3 is characterized by the sequence disclosed in the NCBI Data Bank with the Accession no.: NP_937762.1. Its cDNA sequence is:

(SEQ ID NO: 7)
ATGGCGGCGGCCGCGCGGCCTCGGGGTCGGGCACTGGGGCCAGTACTGCC
GCCGACGCCGCTGCTCCTGCTGGTACTGCGGGTGCTGCCAGCCTGCGGGG
CGACCGCTCGGGATCCCGGGGCCGCGGCCGGGCTCAGCCTTCACCCGACT
TACTTCAACCTGGCCGAGGCGGCGAGGATTTGGGCCACCGCCACCTGCGG
GGAGAGGGGACCCGGCGAGGGGAGGCCCCAGCCCGAGCTCTACTGCAAGT
TGGTCGGGGGCCCCACCGCCCCAGGCAGCGGCCACACCATCCAGGGCCAG
TTCTGTGACTATTGCAATTCTGAAGACCCCAGGAAAGCACATCCTGTCAC
CAATGCCATCGATGGATCTGAACGTTGGTGGCAAAGCCCTCCCCTGTCCT
CAGGCACACAGTACAACAGAGTCAACCTCACCTTGGATCTGGGGCAGCTC
TTCCATGTGGCCTATATTTTAATCAAATTTGCAAATTCTCCTCGCCCTGA
TCTTTGGGTCTTGGAAAGATCTGTAGACTTTGGAAGCACCTACTCACCAT
GGCAATATTTTGCTCATTCTAAAGTAGACTGTTTAAAAGAATTTGGGCGG
GAGGCAAATATGGCTGTCACCCGGGATGATGATGTACTTTGTGTTACTGA
ATATTCCCGTATTGTACCTTTGGAAAATGGTGAGGTTGTGGTGTCCTTGA
TAAACGGTCGTCCAGGTGCAAAAAATTTTACTTTCTCTCACACCCTGAGG
GAGTTTACCAAGGCAACAAACATCCGCTTGCGTTTTCTTAGAACCAATAC
GCTTCTTGGACACCTCATCTCCAAAGCCCAGCGAGATCCAACTGTCACTC
GGCGGTATTATTACAGCATAAAGGACATCAGCATTGGTGGGCAGTGTGTT
TGCAATGGCCATGCTGAAGTGTGCAATATAAACAATCCTGAAAAACTGTT
TCGGTGTGAATGCCAGCACCACACCTGTGGGGAGACGTGTGATCGCTGCT
GCACAGGGTACAATCAGAGGCGCTGGCGGCCCGCCGCTTGGGAGCAGAGC
CACGAGTGTGAAGCATGCAACTGCCACGGCCATGCCAGCAACTGTTACTA
TGATCCAGATGTTGAGCGGCAGCAGGCAAGCTTGAATACCCAGGGCATCT
ATGCTGGTGGAGGGGTCTGCATTAACTGTCAGCACAACACAGCTGGAGTA
AACTGTGAACAGTGTGCTAAGGGCTATTACCGCCCTTATGGGGTTCCAGT
GGATGCCCCTGATGGCTGCATCCCCTGCAGCTGTGACCCTGAGCATGCGG
ATGGCTGTGAACAGGGTTCAGGCCGCTGTCACTGCAAGCCAAATTTCCAC
GGAGACAACTGTGAGAAGTGTGCAATTGGATACTACAATTTCCCATTTTG
CTTGAGAATTCCCATTTTTCCTGTTTCTACACCAAGTTCAGAAGATCCAG
TAGCTGGAGATATAAAAGGGTGTGACTGTAATCTGGAAGGTGTTCTCCCT
GAAATATGTGATGCCCACGGACGGTGCCTGTGCCGCCCTGGGGTTGAGGG
CCCTCGATGTGATACCTGCCGCTCTGGTTTCTACTCATTCCCTATTTGCC
AAGCCTGCTGGTGTTCAGCCCTTGGATCCTACCAGATGCCCTGCAGCTCA
GTGACTGGACAGTGTGAATGTCGGCCAGGAGTTACAGGACAGCGGTGTGA
CAGGTGTCTCTCAGGAGCTTATGATTTCCCCCACTGCCAAGGTTCCAGCA
GTGCTTGTGACCCAGCTGGTACCATCAACTCCAATTTGGGGTATTGCCAA
TGCAAGCTTCATGTTGAAGGTCCTACTTGTAGCCGCTGCAAACTGTTATA
TTGGAATCTGGACAAAGAAAACCCCAGTGGATGTTCAGAATGCAAGTGCC
ATAAGGCGGGAACAGTGAGTGGAACTGGAGAGTGTAGGCAGGGAGATGGT
GACTGTCACTGCAAGTCCCATGTGGGTGGCGATTCCTGCGACACCTGTGA
AGATGGATATTTTGCTTTGGAAAAGAGCAATTACTTTGGGTGTCAAGGGT
GTCAGTGTGACATTGGTGGGGCATTGTCCTCCATGTGCAGTGGGCCCTCG
GGAGTGTGCCAGTGCCGAGAGCATGTCGTGGGAAAGGTGTGCCAGCGGCC
TGAAAACAACTACTATTTCCCAGATTTGCATCATATGAAGTATGAGATTG
AAGACGGCAGCACACCTAATGGGAGAGACCTTCGATTTGGATTTGATCCG
CTGGCATTTCCTGAGTTTAGCTGGAGAGGATATGCCCAAATGACCTCAGT
ACAGAATGATGTAAGAATAACATTGAATGTAGGGAAGTCAAGTGGCTCCT
TGTTTCGTGTTATTCTGAGATACGTTAACCCTGGAACTGAAGCAGTATCT
GGCCATATAACTATTTATCCATCCTGGGGTGCTGCTCAAAGCAAAGAGAT
CATCTTCCTGCCGAGTAAGGAGCCAGCCTTTGTCACTGTCCCTGGAAATG
GTTTTGCAGACCCATTTTCAATCACACCAGGAATATGGGTTGCTTGTATT
AAGGCAGAAGGAGTCCTTCTGGATTACCTGGTGCTGCTCCCCAGGGACTA
CTATGAAGCCTCTGTACTGCAGCTGCCAGTCACAGAACCATGTGCCTACG
CAGGACCTCCCCAAGAAAATTGCTTACTCTACCAGCATTTGCCAGTGACC
AGATTCCCCTGTACCCTGGCTTGTGAGGCCAGACACTTCCTGCTTGATGG
GGAGCCAAGACCCGTGGCAGTGAGGCAGCCCACACCTGCACACCCTGTCA
TGGTGGACCTCAGCGGGAGAGAGGTGGAATTGCATCTGCGGCTGCGCATC
CCACAGGTTGGCCACTACGTGGTTGTGGTCGAGTATTCCACGGAGGCAGC
TCAGCTGTTTGTGGTTGATGTGAATGTGAAGAGCTCCGGGTCTGTTCTGG
CAGGCCAGGTGAACATTTACAGCTGCAACTACAGTGTTCTCTGCCGGAGT
GCTGTGATTGATCACATGAGCCGCATCGCCATGTATGAGCTATTGGCAGA
TGCAGACATTCAGCTCAAGGGACACATGGCCCGATTCCTTCTGCATCAAG
TTTGTATCATACCTATTGAAGAATTCTCAGCTGAGTATGTGAGACCACAA
GTCCACTGCATTGCCAGTTATGGGCGATTTGTCAATCAAAGTGCCACCTG
TGTCTCCTTGGCCCATGAAACTCCTCCAACAGCATTAATTTTGGATGTTC
TAAGTGGCAGGCCTTTCCCTCACCTGCCCCAGCAGTCGTCACCTTCTGTT
GATGTTCTTCCTGGGGTCACCTTGAAGGCACCGCAGAATCAAGTGACCCT
GAGAGGACGTGTACCACACCTGGGCCGATACGTCTTTGTCATCCATTTTT
ACCAAGCAGCGCACCCGACGTTTCCCGCGCAGGTGTCGGTGGATGGCGGG
TGGCCACGGGCAGGCTCCTTCCATGCCTCTTTTTGCCCCCATGTGCTTGG
CTGCCGGGATCAAGTGATTGCCGAAGGCCAGATTGAGTTTGACATCTCAG
AGCCTGAAGTGGCCGCAACTGTGAAGGTTCCAGAAGGAAAGTCCTTGGTT
TTGGTCCGTGTTCTAGTGGTGCCTGCAGAAAACTATGACTACCAAATACT
TCACAAAAAATCCATGGACAAGTCACTCGAGTTTATCACCAATTGTGGAA
AAAACAGCTTTTACCTTGACCCCCAGACAGCCTCCAGATTCTGTAAGAAT
TCCGCCAGGTCCCTGGTGGCCTTTTACCACAAGGGCGCCCTGCCTTGTGA
GTGCCACCCCACTGGGGCCACCGGCCCTCACTGCAGCCCTGAGGGTGGGC
AGTGCCCATGCCAGCCCAACGTCATCGGGCGGCAGTGCACCCGCTGTGCA
ACAGGCCACTACGGATTCCCACGCTGCAAGCCGTGCAGCTGTGGTCGGCG
CCTTTGTGAAGAGATGACGGGGCAGTGCCGCTGCCCTCCCCGCACGGTCA
GGCCCCAGTGTGAGGTGTGTGAGACACACTCATTCAGCTTCCACCCCATG
GCCGGCTGCGAAGGCTGCAACTGTTCCAGGAGGGGCACCATCGAGGCTGC
CATGCCGGAGTGTGACCGGGACAGCGGGCAGTGCAGATGCAAGCCCAGAA
TCACAGGGCGGCAGTGTGACCGATGTGCTTCCGGGTTTTACCGCTTTCCT
GAGTGTGTTCCCTGCAATTGCAACAGAGATGGGACTGAGCCAGGAGTGTG
TGACCCAGGGACCGGGGCTTGCCTCTGCAAGGAAAATGTAGAAGGCACAG
AGTGTAATGTGTGTCGAGAAGGCTCATTCCATTTGGACCCAGCCAATCTC
AAGGGTTGTACCAGCTGTTTCTGTTTTGGAGTAAATAATCAATGTCACAG
CTCACATAAGCGAAGGACTAAGTTTGTGGATATGCTGGGCTGGCACCTGG
AGACAGCAGACAGAGTGGACATCCCTGTCTCTTTCAACCCAGGCAGCAAC
AGTATGGTGGCGGATCTCCAGGAGCTGCCCGCAACCATCCACAGCGCGTC
CTGGGTCGCACCCACCTCCTACCTGGGGGACAAGGTTTCTTCATATGGTG
GTTACCTCACTTACCAAGCCAAGTCCTTTGGCTTGCCTGGCGACATGGTT
CTTCTGGAAAAGAAGCCGGATGTACAGCTCACTGGTCAGCACATGTCCAT
CATCTATGAGGAGACAAACACCCCACGGCCAGACCGGCTGCATCATGGAC
GAGTGCACGTGGTCGAGGGAAACTTCAGACATGCCAGCAGCCGTGCCCCA
GTGTCTAGGGAGGAGCTGATGACAGTGCTGTCTAGACTGGCAGATGTGCG
CATCCAAGGCCTCTACTTCACAGAGACTCAAAGGCTCACCCTGAGCGAGG
TGGGGCTAGAGGAAGCCTCTGACACAGGAAGTGGGCGCATAGCACTTGCT
GTGGAAATCTGTGCCTGCCCCCCTGCCTACGCTGGTGACTCTTGTCAGGG
TTGTAGCCCTGGATACTATCGGGATCATAAAGGCTTGTATACCGGACGGT
GTGTTCCCTGCAATTGCAACGGACATTCAAATCAATGCCAGGATGGCTCA
GGCATATGTGTTAACTGTCAGCACAACACCGCGGGAGAGCACTGTGAACG
CTGCCAGGAGGGCTACTATGGCAACGCCGTCCACGGATCCTGCAGGGCCT
GCCCATGTCCTCACACTAACAGCTTTGCCACTGGCTGTGTGGTGAATGGG
GGAGACGTGCGGTGCTCCTGCAAAGCTGGGTACACAGGAACACAGTGTGA
AAGGTGTGCACCGGGATATTTCGGGAATCCCCAGAAATTCGGAGGTAGCT
GCCAACCATGCAGTTGTAACAGCAATGGCCAGCTGGGCAGCTGTCATCCC
CTGACTGGAGACTGCATAAACCAAGAACCCAAAGATAGCAGCCCTGCAGA
AGAATGTGATGATTGCGACAGCTGTGTGATGACCCTCCTGAACGACCTGG
CCACCATGGGCGAGCAGCTCCGCCTGGTCAAGTCTCAGCTGCAGGGCCTG
AGTGCCAGCGCAGGGCTTCTGGAGCAGATGAGGCACATGGAGACCCAGGC
CAAGGACCTGAGGAATCAGTTGCTCAACTACCGTTCTGCCATTTCAAATC
ATGGATCAAAAATAGAAGGCCTGGAAAGAGAACTGACTGATTTGAATCAA
GAATTTGAGACTTTGCAAGAAAAGGCTCAAGTAAATTCCAGAAAAGCACA
AACATTAAACAACAATGTTAATCGGGCAACACAAAGCGCAAAAGAACTGG
ATGTGAAGATTAAAAATGTCATCCGGAATGTGCACATGCTGAACCGGATA
AGGACCTGGCAGAAAACCCACCAGGGGGAGAACAATGGGCTTGCTAACAG
TATCCGGGATTCTTTAAATGAATACGAAGCCAAACTCAGTGACCTTCGTG
CTCGGCTGCAGGAGGCAGCTGCCCAAGCCAAGCAGGCAAATGGCTTGAAC
CAAGAAAACGAGAGAGCTTTGGGAGCCATTCAGAGACAAGTGAAAGAAAT
AAATTCCCTGCAGAGTGATTTCACCAAGTATCTAACCACTGCAGACTCAT
CTTTGTTGCAAACCAACATTGCGCTGCAGCTGATGGAGAAAAGCCAGAAG
GAATATGAAAAATTAGCTGCCAGTTTAAATGAAGCAAGACAAGAACTAAG
TGACAAAGTAAGAGAACTTTCCAGATCTGCTGGCAAAACATCCCTTGTGG
AGGAGGCAGAAAAGCACGCGCGGTCCTTACAAGAGCTGGCAAAGCAGCTG
GAAGAGATCAAGAGAAACGCCAGCGGGGATGAGCTGGTGCGCTGTGCTGT
GGATGCCGCCACCGCCTACGAGAACATCCTCAATGCCATCAAAGCGGCCG
AGGACGCAGCCAACAGGGCTGCCAGTGCATCTGAATCTGCCCTCCAGACA
GTGATAAAGGAAGATCTGCCAAGAAAAGCTAAAACCCTGAGTTCCAACAG
TGATAAACTGTTAAATGAAGCCAAGATGACACAAAAGAAGCTAAAGCAAG
AAGTCAGTCCAGCTCTCAACAACCTACAGCAAACCCTGAATATTGTGACA
GTTCAGAAAGAAGTGATAGACACCAATCTCACAACTCTCCGAGATGGTCT
TCATGGGATACAGAGAGGTGATATTGATGCTATGATCAGTAGTGCAAAGA
GCATGGTCAGAAAGGCCAACGACATCACAGATGAGGTTCTGGATGGGCTC
AACCCCATCCAGACAGATGTGGAAAGAATTAAGGACACCTATGGGAGGAC
ACAGAACGAAGACTTCAAAAAGGCTCTGACTGATGCAGATAACTCGGTGA
ATAAGTTAACCAACAAACTACCTGATCTTTGGCGCAAGATTGAAAGTATC
AACCAACAGCTGTTGCCCTTGGGAAACATCTCTGACAACATGGACAGAAT
ACGAGAACTAATTCAGCAGGCCAGAGATGCTGCCAGTAAGGTTGCTGTCC
CCATGAGGTTCAATGGTAAATCTGGAGTCGAAGTCCGACTGCCAAATGAC
CTGGAAGATTTGAAAGGATATACATCTCTGTCCTTGTTTCTCCAAAGGCC
CAACTCAAGAGAAAATGGGGGTACTGAGAATATGTTTGTGATGTACCTTG
GAAATAAAGATGCCTCCCGGGACTACATCGGCATGGCAGTTGTGGATGGC
CAGCTCACCTGTGTCTACAACCTGGGGGACCGTGAGGCTGAACTCCAAGT
GGACCAGATCTTGACCAAGAGTGAGACTAAGGAGGCAGTTATGGATCGGG
TGAAATTTCAGAGAATTTATCAGTTTGCAAGGCTTAATTACACCAAAGGA
GCCACATCCAGTAAACCAGAAACACCCGGAGTCTATGACATGGATGGTAG
AAATAGCAATACACTCCTTAATTTGGATCCTGAAAATGTTGTATTTTATG
TTGGAGGTTACCCACCTGATTTTAAACTTCCCAGTCGACTAAGTTTCCCT
CCATACAAAGGTTGTATTGAATTAGATGACCTCAATGAAAATGTTCTGAG
CTTGTACAACTTCAAAAAAACATTCAATCTCAACACAACTGAAGTGGAGC
CTTGTAGAAGGAGGAAGGAAGAGTCAGACAAAAATTATTTTGAAGGTACG
GGCTATGCTCGAGTTCCAACTCAACCACATGCTCCCATCCCAACCTTTGG
ACAGACAATTCAGACCACCGTGGATAGAGGCTTGCTGTTCTTTGCAGAAA
ACGGGGATCGCTTCATATCTCTAAATATAGAAGATGGCAAGCTCATGGTG
AGATACAAACTGAATTCAGAGCTACCAAAAGAGAGAGGAGTTGGAGACGC
CATAAACAACGGCAGAGACCATTCGATTCAGATCAAAATTGGAAAACTCC
AAAAGCGTATGTGGATAAATGTGGACGTTCAAAACACTATAATTGATGGT
GAAGTATTTGATTTCAGCACATATTATCTGGGAGGAATTCCAATTGCAAT
CAGGGAAAGATTTAACATTTCTACGCCTGCTTTCCGAGGCTGCATGAAAA
ATTTGAAGAAAACCAGTGGTGTCGTTAGATTGAATGATACTGTGGGAGTA
ACCAAAAAGTGCTCGGAAGACTGGAAGCTTGTGCGATCTGCCTCATTCTC
CAGAGGAGGACAATTGAGTTTCACTGATTTGGGCTTACCACCTACTGACC
ACCTCCAGGCCTCATTTGGATTTCAGACCTTTCAACCCAGTGGCATATTA
TTAGATCATCAGACATGGACAAGGAACCTGCAGGTCACTCTGGAAGATGG
TTACATTGAATTGAGCACCAGCGATAGCGGCGGCCCAATTTTTAAATCTC
CACAGACGTATATGGATGGTTTACTGCATTATGTATCTGTAATAAGCGAC
AACTCTGGACTACGGCTTCTCATCGATGACCAGCTTCTGAGAAATAGCAA
AAGGCTAAAACACATTTCAAGTTCCCGGCAGTCTCTGCGTCTGGGCGGGA
GCAATTTTGAGGGTTGTATTAGCAATGTTTTTGTCCAGAGGTTATCACTG
AGTCCTGAAGTCCTAGATTTGACCAGTAACTCTCTCAAGAGAGATGTGTC
CCTGGGAGGCTGCAGTTTAAACAAACCACCTTTTCTAATGTTGCTTAAAG
GTTCTACCAGGTTTAACAAGACCAAGACTTTTCGTATCAACCAGCTGTTG
CAGGACACACCAGTGGCCTCCCCAAGGAGCGTGAAGGTGTGGCAAGATGC
TTGCTCACCACTTCCCAAGACCCAGGCCAATCATGGAGCCCTCCAGTTTG
GGGACATTCCCACCAGCCACTTGCTATTCAAGCTTCCTCAGGAGCTGCTG
AAACCCAGGTCACAGTTTGCTGTGGACATGCAGACAACATCCTCCAGAGG
ACTGGTGTTTCACACGGGCACTAAGAACTCCTTTATGGCTCTTTATCTTT
CAAAAGGACGTCTGGTCTTTGCACTGGGGACAGATGGGAAAAAATTGAGG
ATCAAAAGCAAGGAGAAATGCAATGATGGGAAATGGCACACGGTGGTGTT
TGGCCATGATGGGGAAAAGGGGCGCTTGGTTGTGGATGGACTGAGGGCCC
GGGAGGGAAGTTTGCCTGGAAACTCCACCATCAGCATCAGAGCGCCAGTT
TACCTGGGATCACCTCCATCAGGGAAACCAAAGAGCCTCCCCACAAACAG
CTTTGTGGGATGCCTGAAGAACTTTCAGCTGGATTCAAAACCCTTGTATA
CCCCTTCTTCAAGCTTCGGGGTGTCTTCCTGCTTGGGTGGTCCTTTGGAG
AAAGGCATTTATTTCTCTGAAGAAGGAGGTCATGTCGTCTTGGCTCACTC
TGTATTGTTGGGGCCAGAATTTAAGCTTGTTTTCAGCATCCGCCCAAGAA
GTCTCACTGGGATCCTAATACACATCGGAAGTCAGCCCGGGAAGCACTTA
TGTGTTTACCTGGAGGCAGGAAAGGTCACGGCCTCTATGGACAGTGGGGC
AGGTGGGACCTCAACGTCGGTCACACCAAAGCAGTCTCTGTGTGATGGAC
AGTGGCACTCGGTGGCAGTCACCATAAAACAACACATCCTGCACCTGGAA
CTGGACACAGACAGTAGCTACACAGCTGGACAGATCCCCTTCCCACCTGC
CAGCACTCAAGAGCCACTACACCTTGGAGGTGCTCCAGCCAATTTGACGA
CACTGAGGATCCCTGTGTGGAAATCATTCTTTGGCTGTCTGAGGAATATT
CATGTCAATCACATCCCTGTCCCTGTCACTGAAGCCTTGGAAGTCCAGGG
GCCTGTCAGTCTGAATGGTTGTCCTGACCAGTAA

Its protein sequence is (3333 aa):

(SEQ ID NO: 8)
MAAAARPRGRALGPVLPPTPLLLLVLRVLPACGATARDPGAAAGLSLHPT
YFNLAEAARIWATATCGERGPGEGRPQPELYCKLVGGPTAPGSGHTIQGQ
FCDYCNSEDPRKAHPVTNAIDGSERWWQSPPLSSGTQYNRVNLTLDLGQL
FHVAYILIKFANSPRPDLWVLERSVDFGSTYSPWQYFAHSKVDCLKEFGR
EANMAVTRDDDVLCVTEYSRIVPLENGEVVVSLINGRPGAKNFTFSHTLR
EFTKATNIRLRFLRTNTLLGHLISKAQRDPTVTRRYYYSIKDISIGGQCV
CNGHAEVCNINNPEKLFRCECQHHTCGETCDRCCTGYNQRRWRPAAWEQS
HECEACNCHGHASNCYYDPDVERQQASLNTQGIYAGGGVCINCQHNTAGV
NCEQCAKGYYRPYGVPVDAPDGCIPCSCDPEHADGCEQGSGRCHCKPNFH
GDNCEKCAIGYYNFPFCLRIPIFPVSTPSSEDPVAGDIKGCDCNLEGVLP
EICDAHGRCLCRPGVEGPRCDTCRSGFYSFPICQACWCSALGSYQMPCSS
VTGQCECRPGVTGQRCDRCLSGAYDFPHCQGSSSACDPAGTINSNLGYCQ
CKLHVEGPTCSRCKLLYWNLDKENPSGCSECKCHKAGTVSGTGECRQGDG
DCHCKSHVGGDSCDTCEDGYFALEKSNYFGCQGCQCDIGGALSSMCSGPS
GVCQCREHVVGKVCQRPENNYYFPDLHHMKYEIEDGSTPNGRDLRFGFDP
LAFPEFSWRGYAQMTSVQNDVRITLNVGKSSGSLFRVILRYVNPGTEAVS
GHITIYPSWGAAQSKEIIFLPSKEPAFVTVPGNGFADPFSITPGIWVACI
KAEGVLLDYLVLLPRDYYEASVLQLPVTEPCAYAGPPQENCLLYQHLPVT
RFPCTLACEARHFLLDGEPRPVAVRQPTPAHPVMVDLSGREVELHLRLRI
PQVGHYVVVVEYSTEAAQLFVVDVNVKSSGSVLAGQVNIYSCNYSVLCRS
AVIDHMSRIAMYELLADADIQLKGHMARFLLHQVCIIPIEEFSAEYVRPQ
VHCIASYGRFVNQSATCVSLAHETPPTALILDVLSGRPFPHLPQQSSPSV
DVLPGVTLKAPQNQVTLRGRVPHLGRYVFVIHFYQAAHPTFPAQVSVDGG
WPRAGSFHASFCPHVLGCRDQVIAEGQIEFDISEPEVAATVKVPEGKSLV
LVRVLVVPAENYDYQILHKKSMDKSLEFITNCGKNSFYLDPQTASRFCKN
SARSLVAFYHKGALPCECHPTGATGPHCSPEGGQCPCQPNVIGRQCTRCA
TGHYGFPRCKPCSCGRRLCEEMTGQCRCPPRTVRPQCEVCETHSFSFHPM
AGCEGCNCSRRGTIEAAMPECDRDSGQCRCKPRITGRQCDRCASGFYRFP
ECVPCNCNRDGTEPGVCDPGTGACLCKENVEGTECNVCREGSFHLDPANL
KGCTSCFCFGVNNQCHSSHKRRTKFVDMLGWHLETADRVDIPVSFNPGSN
SMVADLQELPATIHSASWVAPTSYLGDKVSSYGGYLTYQAKSFGLPGDMV
LLEKKPDVQLTGQHMSIIYEETNTPRPDRLHHGRVHVVEGNFRHASSRAP
VSREELMTVLSRLADVRIQGLYFTETQRLTLSEVGLEEASDTGSGRIALA
VEICACPPAYAGDSCQGCSPGYYRDHKGLYTGRCVPCNCNGHSNQCQDGS
GICVNCQHNTAGEHCERCQEGYYGNAVHGSCRACPCPHTNSFATGCVVNG
GDVRCSCKAGYTGTQCERCAPGYFGNPQKFGGSCQPCSCNSNGQLGSCHP
LTGDCINQEPKDSSPAEECDDCDSCVMTLLNDLATMGEQLRLVKSQLQGL
SASAGLLEQMRHMETQAKDLRNQLLNYRSAISNHGSKIEGLERELTDLNQ
EFETLQEKAQVNSRKAQTLNNNVNRATQSAKELDVKIKNVIRNVHILLKQ
ISGTDGEGNNVPSGDFSREWAEAQRMMRELRNRNFGKHLREAEADKRESQ
LLLNRIRTWQKTHQGENNGLANSIRDSLNEYEAKLSDLRARLQEAAAQAK
QANGLNQENERALGAIQRQVKEINSLQSDFTKYLTTADSSLLQTNIALQL
MEKSQKEYEKLAASLNEARQELSDKVRELSRSAGKTSLVEEAEKHARSLQ
ELAKQLEEIKRNASGDELVRCAVDAATAYENILNAIKAAEDAANRAASAS
ESALQTVIKEDLPRKAKTLSSNSDKLLNEAKMTQKKLKQEVSPALNNLQQ
TLNIVTVQKEVIDTNLTTLRDGLHGIQRGDIDAMISSAKSMVRKANDITD
EVLDGLNPIQTDVERIKDTYGRTQNEDFKKALTDADNSVNKLTNKLPDLW
RKIESINQQLLPLGNISDNMDRIRELIQQARDAASKVAVPMRFNGKSGVE
VRLPNDLEDLKGYTSLSLFLQRPNSRENGGTENMFVMYLGNKDASRDYIG
MAVVDGQLTCVYNLGDREAELQVDQILTKSETKEAVMDRVKFQRIYQFAR
LNYTKGATSSKPETPGVYDMDGRNSNTLLNLDPENVVFYVGGYPPDFKLP
SRLSFPPYKGCIELDDLNENVLSLYNFKKTFNLNTTEVEPCRRRKEESDK
NYFEGTGYARVPTQPHAPIPTFGQTIQTTVDRGLLFFAENGDRFISLNIE
DGKLMVRYKLNSELPKERGVGDAINNGRDHSIQIKIGKLQKRMWINVDVQ
NTIIDGEVFDFSTYYLGGIPIAIRERFNISTPAFRGCMKNLKKTSGVVRL
NDTVGVTKKCSEDWKLVRSASFSRGGQLSFTDLGLPPTDHLQASFGFQTF
QPSGILLDHQTWTRNLQVTLEDGYIELSTSDSGGPIFKSPQTYMDGLLHY
VSVISDNSGLRLLIDDQLLRNSKRLKHISSSRQSLRLGGSNFEGCISNVF
VQRLSLSPEVLDLTSNSLKRDVSLGGCSLNKPPFLMLLKGSTRFNKTKTF
RINQLLQDTPVASPRSVKVWQDACSPLPKTQANHGALQFGDIPTSHLLFK
LPQELLKPRSQFAVDMQTTSSRGLVFHTGTKNSFMALYLSKGRLVFALGT
DGKKLRIKSKEKCNDGKWHTVVFGHDGEKGRLVVDGLRAREGSLPGNSTI
SIRAPVYLGSPPSGKPKSLPTNSFVGCLKNFQLDSKPLYTPSSSFGVSSC
LGGPLEKGIYFSEEGGHVVLAHSVLLGPEFKLVFSIRPRSLTGILIHIGS
QPGKHLCVYLEAGKVTASMDSGAGGTSTSVTPKQSLCDGQWHSVAVTIKQ
HILHLELDTDSSYTAGQIPFPPASTQEPLHLGGAPANLTTLRIPVWKSFF
GCLRNIHVNHIPVPVTEALEVQGPVSLNGCPDQ

Preferably, the LAMC2 is characterized by the sequence disclosed in the NCBI Data Bank with the Accession no.: NM_018891. Its cDNA sequence is:

(SEQ ID NO: 9)
ATGCCTGCGCTCTGGCTGGGCTGCTGCCTCTGCTTCTCGCTCCTCCTGCC
CGCAGCCCGGGCCACCTCCAGGAGGGAAGTCTGTGATTGCAATGGGAAGT
CCAGGCAGTGTATCTTTGATCGGGAACTTCACAGACAAACTGGTAATGGA
TTCCGCTGCCTCAACTGCAATGACAACACTGATGGCATTCACTGCGAGAA
GTGCAAGAATGGCTTTTACCGGCACAGAGAAAGGGACCGCTGTTTGCCCT
GCAATTGTAACTCCAAAGGTTCTCTTAGTGCTCGATGTGACAACTCCGGA
CGGTGCAGCTGTAAACCAGGTGTGACAGGAGCCAGATGCGACCGATGTCT
GCCAGGCTTCCACATGCTCACGGATGCGGGGTGCACCCAAGACCAGAGAC
TGCTAGACTCCAAGTGTGACTGTGACCCAGCTGGCATCGCAGGGCCCTGT
GACGCGGGCCGCTGTGTCTGCAAGCCAGCTGTCACTGGAGAACGCTGTGA
TAGGTGTCGATCAGGTTACTATAATCTGGATGGGGGGAACCCTGAGGGCT
GTACCCAGTGTTTCTGCTATGGGCATTCAGCCAGCTGCCGCAGCTCTGCA
GAATACAGTGTCCATAAGATCACCTCTACCTTTCATCAAGATGTTGATGG
CTGGAAGGCTGTCCAACGAAATGGGTCTCCTGCAAAGCTCCAATGGTCAC
AGCGCCATCAAGATGTGTTTAGCTCAGCCCAACGACTAGACCCTGTCTAT
TTTGTGGCTCCTGCCAAATTTCTTGGGAATCAACAGGTGAGCTATGGTCA
AAGCCTGTCCTTTGACTACCGTGTGGACAGAGGAGGCAGACACCCATCTG
CCCATGATGTGATTCTGGAAGGTGCTGGTCTACGGATCACAGCTCCCTTG
ATGCCACTTGGCAAGACACTGCCTTGTGGGCTCACCAAGACTTACACATT
CAGGTTAAATGAGCATCCAAGCAATAATTGGAGCCCCCAGCTGAGTTACT
TTGAGTATCGAAGGTTACTGCGGAATCTCACAGCCCTCCGCATCCGAGCT
ACATATGGAGAATACAGTACTGGGTACATTGACAATGTGACCCTGATTTC
AGCCCGCCCTGTCTCTGGAGCCCCAGCACCCTGGGTTGAACAGTGTATAT
GTCCTGTTGGGTACAAGGGGCAATTCTGCCAGGATTGTGCTTCTGGCTAC
AAGAGAGATTCAGCGAGACTGGGGCCTTTTGGCACCTGTATTCCTTGTAA
CTGTCAAGGGGGAGGGGCCTGTGATCCAGACACAGGAGATTGTTATTCAG
GGGATGAGAATCCTGACATTGAGTGTGCTGACTGCCCAATTGGTTTCTAC
AACGATCCGCACGACCCCCGCAGCTGCAAGCCATGTCCCTGTCATAACGG
GTTCAGCTGCTCAGTGATGCCGGAGACGGAGGAGGTGGTGTGCAATAACT
GCCCTCCCGGGGTCACCGGTGCCCGCTGTGAGCTCTGTGCTGATGGCTAC
TTTGGGGACCCCTTTGGTGAACATGGCCCAGTGAGGCCTTGTCAGCCCTG
TCAATGCAACAACAATGTGGACCCCAGTGCCTCTGGGAATTGTGACCGGC
TGACAGGCAGGTGTTTGAAGTGTATCCACAACACAGCCGGCATCTACTGC
GACCAGTGCAAAGCAGGCTACTTCGGGGACCCATTGGCTCCCAACCCAGC
AGACAAGTGTCGAGCTTGCAACTGTAACCCCATGGGCTCAGAGCCTGTAG
GATGTCGAAGTGATGGCACCTGTGTTTGCAAGCCAGGATTTGGTGGCCCC
AACTGTGAGCATGGAGCATTCAGCTGTCCAGCTTGCTATAATCAAGTGAA
GATTCAGATGGATCAGTTTATGCAGCAGCTTCAGAGAATGGAGGCCCTGA
TTTCAAAGGCTCAGGGTGGTGATGGAGTAGTACCTGATACAGAGCTGGAA
GGCAGGATGCAGCAGGCTGAGCAGGCCCTTCAGGACATTCTGAGAGATGC
CCAGATTTCAGAAGGTGCTAGCAGATCCCTTGGTCTCCAGTTGGCCAAGG
TGAGGAGCCAAGAGAACAGCTACCAGAGCCGCCTGGATGACCTCAAGATG
ACTGTGGAAAGAGTTCGGGCTCTGGGAAGTCAGTACCAGAACCGAGTTCG
GGATACTCACAGGCTCATCACTCAGATGCAGCTGAGCCTGGCAGAAAGTG
AAGCTTCCTTGGGAAACACTAACATTCCTGCCTCAGACCACTACGTGGGG
CCAAATGGCTTTAAAAGTCTGGCTCAGGAGGCCACAAGATTAGCAGAAAG
CCACGTTGAGTCAGCCAGTAACATGGAGCAACTGACAAGGGAAACTGAGG
ACTATTCCAAACAAGCCCTCTCACTGGTGCGCAAGGCCCTGCATGAAGGA
GTCGGAAGCGGAAGCGGTAGCCCGGACGGTGCTGTGGTGCAAGGGCTTGT
GGAAAAATTGGAGAAAACCAAGTCCCTGGCCCAGCAGTTGACAAGGGAGG
CCACTCAAGCGGAAATTGAAGCAGATAGGTCTTATCAGCACAGTCTCCGC
CTCCTGGATTCAGTGTCTCGGCTTCAGGGAGTCAGTGATCAGTCCTTTCA
GGTGGAAGAAGCAAAGAGGATCAAACAAAAAGCGGATTCACTCTCAAGCC
TGGTAACCAGGCATATGGATGAGTTCAAGCGTACACAGAAGAATCTGGGA
AACTGGAAAGAAGAAGCACAGCAGCTCTTACAGAATGGAAAAAGTGGGAG
AGAGAAATCAGATCAGCTGCTTTCCCGTGCCAATCTTGCTAAAAGCAGAG
CACAAGAAGCACTGAGTATGGGCAATGCCACTTTTTATGAAGTTGAGAGC
ATCCTTAAAAACCTCAGAGAGTTTGACCTGCAGGTGGACAACAGAAAAGC
AGAAGCTGAAGAAGCCATGAAGAGACTCTCCTACATCAGCCAGAAGGTTT
CAGATGCCAGTGACAAGACCCAGCAAGCAGAAAGAGCCCTGGGGAGCGCT
GCTGCTGATGCACAGAGGGCAAAGAATGGGGCCGGGGAGGCCCTGGAAAT
CTCCAGTGAGATTGAACAGGAGATTGGGAGTCTGAACTTGGAAGCCAATG
TGACAGCAGATGGAGCCTTGGCCATGGAAAAGGGACTGGCCTCTCTGAAG
AGTGAGATGAGGGAAGTGGAAGGAGAGCTGGAAAGGAAGGAGCTGGAGTT
TGACACGAATATGGATGCAGTACAGATGGTGATTACAGAAGCCCAGAAGG
TTGATACCAGAGCCAAGAACGCTGGGGTTACAATCCAAGACACACTCAAC
ACATTAGACGGCCTCCTGCATCTGATGGGTATGTGA

Its protein sequence is:

(SEQ ID NO: 10)
MPALWLGCCLCFSLLLPAARATSRREVCDCNGKSRQCIFDRELHRQTGNG
FRCLNCNDNTDGIHCEKCKNGFYRHRERDRCLPCNCNSKGSLSARCDNSG
RCSCKPGVTGARCDRCLPGFHMLTDAGCTQDQRLLDSKCDCDPAGIAGPC
DAGRCVCKPAVTGERCDRCRSGYYNLDGGNPEGCTQCFCYGHSASCRSSA
EYSVHKITSTFHQDVDGWKAVQRNGSPAKLQWSQRHQDVFSSAQRLDPVY
FVAPAKFLGNQQVSYGQSLSFDYRVDRGGRHPSAHDVILEGAGLRITAPL
MPLGKTLPCGLTKTYTFRLNEHPSNNWSPQLSYFEYRRLLRNLTALRIRA
TYGEYSTGYIDNVTLISARPVSGAPAPWVEQCICPVGYKGQFCQDCASGY
KRDSARLGPFGTCIPCNCQGGGACDPDTGDCYSGDENPDIECADCPIGFY
NDPHDPRSCKPCPCHNGFSCSVMPETEEVVCNNCPPGVTGARCELCADGY
FGDPFGEHGPVRPCQPCQCNNNVDPSASGNCDRLTGRCLKCIHNTAGIYC
DQCKAGYFGDPLAPNPADKCRACNCNPMGSEPVGCRSDGTCVCKPGFGGP
NCEHGAFSCPACYNQVKIQMDQFMQQLQRMEALISKAQGGDGVVPDTELE
GRMQQAEQALQDILRDAQISEGASRSLGLQLAKVRSQENSYQSRLDDLKM
TVERVRALGSQYQNRVRDTHRLITQMQLSLAESEASLGNTNIPASDHYVG
PNGFKSLAQEATRLAESHVESASNMEQLTRETEDYSKQALSLVRKALHEG
VGSGSGSPDGAVVQGLVEKLEKTKSLAQQLTREATQAEIEADRSYQHSLR
LLDSVSRLQGVSDQSFQVEEAKRIKQKADSLSSLVTRHMDEFKRTQKNLG
NWKEEAQQLLQNGKSGREKSDQLLSRANLAKSRAQEALSMGNATFYEVES
ILKNLREFDLQVDNRKAEAEEAMKRLSYISQKVSDASDKTQQAERALGSA
AADAQRAKNGAGEALEISSEIEQEIGSLNLEANVTADGALAMEKGLASLK
SEMREVEGELERKELEFDTNMDAVQMVITEAQKVDTRAKNAGVTIQDTLN
TLDGLLHLMGM

Included in the present invention are also nucleic acid sequences derived from the sequences shown below, e.g. functional fragments, mutants, derivatives, analogues, and sequences having a % of identity of at least 70% with the below sequences.

In the context of the present invention, the cDNA, the gene, the mRNA, the polynucleotide or the protein encoded therefrom herein mentioned comprise also their functional fragments, functional analogous, derivatives, variants, isoforms, orthologues or homologous, splicing variants, functional mutants, etc.

The term gene (or cDNA) herein also includes corresponding orthologous or homologous genes, isoforms, variants, allelic variants, functional derivatives, functional fragments thereof. The expression “protein” is intended to include also the corresponding protein encoded from a corresponding orthologous or homologous genes, functional mutants, functional derivatives, functional fragments or analogues, isoforms thereof.

In the context of the present invention, the term “polypeptide” or “protein” includes:

i. the whole protein, allelic variants and orthologs thereof;

ii. any synthetic, recombinant or proteolytic functional fragment;

iii. any functional equivalent, such as, for example, synthetic or recombinant functional analogues.

In the present invention “functional mutants” of the protein are mutants that may be generated by mutating one or more amino acids in their sequences and that maintain their activity. Indeed, the protein of the invention, if required, can be modified in vitro and/or in vivo, for example by glycosylation, myristoylation, amidation, carboxylation or phosphorylation, and may be obtained, for example, by synthetic or recombinant techniques known in the art. The term “derivative” as used herein in relation to a protein means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters and the like. As used herein, the term “derivatives” also refers to longer or shorter polypeptides having e.g. a percentage of identity of at least 41%, preferably at least 41.5%, 50%, 54.9%, 60%, 61.2%, 64.1%, 65%, 70% or 75%, more preferably of at least 85%, as an example of at least 90%, and even more preferably of at least 95% with the herein disclosed genes and sequences, or with an amino acid sequence of the correspondent region encoded from orthologous or homologous gene thereof. The term “analogue” as used herein referring to a protein means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. A “derivative” may be a nucleic acid molecule, as a DNA molecule, coding the polynucleotide as above defined, or a nucleic acid molecule comprising the polynucleotide as above defined, or a polynucleotide of complementary sequence. In the context of the present invention the term “derivatives” also refers to longer or shorter polynucleotides and/or polynucleotides having e.g. a percentage of identity of at least 41%, 50%, 60%, 65%, 70% or 75%, more preferably of at least 85%, as an example of at least 90%, and even more preferably of at least 95% or 100% with the sequences herein discloses or with their complementary sequence or with their DNA or RNA corresponding sequence. The term “derivatives” and the term “polynucleotide” also include modified synthetic oligonucleotides. The modified synthetic oligonucleotide are preferably LNA (Locked Nucleic Acid), phosphoro-thiolated oligos or methylated oligos, morpholinos, 2′-O-methyl, 2′-O-methoxyethyl oligonucleotides and cholesterol-conjugated 2′-O-methyl modified oligonucleotides (antagomirs). The term “derivative” may also include nucleotide analogues, i.e. a naturally occurring ribonucleotide or deoxyribonucleotide substituted by a non-naturally occurring nucleotide. The term “derivatives” also includes nucleic acids or polypeptides that may be generated by mutating one or more nucleotide or amino acid in their sequences, equivalents or precursor sequences. The term “derivatives” also includes at least one functional fragment of the polynucleotide. In the context of the present invention “functional” is intended for example as “maintaining their activity”. As used herein “fragments” refers to polynucleotides having preferably a length of at least 1000 nucleotides, 1100 nucleotide, 1200 nucleotides, 1300 nucleotides, 1400 nucleotides, 1500 nucleotides or to polypeptide having preferably a length of at least 50 aa, 100 aa, 150 aa, 200 aa, 250 aa, 300 aa., . . . . The term “polynucleotide” also refers to modified polynucleotides.

The term “functional fragment” or “functional derivative” may be understood as maintaining the same activity of the protein. “Derivatives” may be recombinant or synthetic. The term “derivative” as used herein in relation to a protein means a chemically modified protein or an analogue thereof, wherein at least one substituent is not present in the unmodified protein or an analogue thereof, i.e. a protein which has been covalently modified. Typical modifications are amides, carbohydrates, alkyl groups, acyl groups, esters and the like

In the context of the present invention, the stratified epithelium above described is preferably epidermis.

Fibrin guarantees a solid-biological substrate to the cells allowing their grown in order to obtain a flap of genetically modified cells adhered to said substrate.

Fibrin is a poorly soluble fraction produced by the specific hydrolysis carried out by the thrombin of the fibrinogen alpha A chain and B beta chain to release fibrinopeptides A and B.

Thrombin is a protease that can act on fibrinogen to produce fibrin. In the composition of the present invention, thrombin may be present in a catalytically effective amount to convert fibrinogen into fibrin. Fibrinogen and thrombin are preferably derived from humans but may also be derived from other animals such as monkey, pig, rat, dog, bovine, etc.

Fibrinogen and thrombin for use in the present invention may be commercially available products.

Preferably, the fibrinogen and thrombin composition of the present invention also includes calcium chloride (which may be in hydrate form), aprotinin, sodium chloride.

An example of the composition of the present invention (for 12 ml total, i.e. 6 ml of fibrinogen mixed with 6 ml of thrombin) consists of: Fibrinogen from 20 to 100 mg/ml, preferably from 20 to 50 mg/ml, more preferably from 20 to 40 mg/ml, even more preferably from 20 to 25 mg/ml;

Thrombin from 1 to 10 IU/ml, preferably from 3 to 8 IU/ml, more preferably from 2 to 4 IU/ml; Aprotinin 1100 IU/ml to 2000 IU/ml;

Buffer consisting ofNacl (1-11%) and CaCl₂ (1-1.5 mM).

Preferably, fibrin gels consist of fibrinogen (23.1 mg/ml) and thrombin (3.1 IU/ml) in NaCl (1%), CaCl₂ (1 mM) and Aprotinin (1,786 KIU/ml).

In a preferred form of the invention, the physiological solution (NaCl 0.9%) is used in the preparation of aprotinin. 10% NaCl is preferably used in the buffer to dissolve fibrinogen and thrombin.

Aprotinin and/or sodium chloride, etc., may be added to the fibrinogen before mixing the composition with the thrombin.

Sodium chloride can be added to the thrombin before mixing the composition with fibrinogen. Before releasing the fibrin gels, they are subjected to conformity controls as per Table 6.

TABLE 6
Features for fibrin gel releasing

| Parameters | Features |
|---|---|
| Transparence | Opacity absence |
| Uniformity | Uniform fibrin gel |
| Structural integrity | Hole absence |

The fibrin composition (including fibrinogen and thrombin) as described above may be used to coat a surface of the support for the preparation of cell flaps. The support may be of any type known to the art expert, provided that the cells can be cultivated on it. Support examples include untreated petri dish plates for cell cultures. Other support examples are culture plates or plates having 6 to 96 wells characterized by being able to facilitate fibrin detachment. Non-limiting examples of support material are: glass, modified glass, polystyrene, ceramic, polymethacrylate and cell culture plates, provided that the material is capable of promoting fibrin detachment. The above-described composition comprising fibrinogen and thrombin is applied to a surface of the support and left at room temperature for 10-15 minutes or until complete polymerization. The support thus obtained can be stored under sterile conditions at 4° C.

The fibrin composition as above defined preferably comprises aprotinin from 1100 KIU/ml to 2000 KIU/ml.

The term “confluence” in the context of the present invention indicates preferably the state in which the cells have such a density that there is no space among them and can be evaluated by the microscope.

The term “subconfluence” in the context of the present invention indicates preferably the state in which the optionally genetically modified cells, e.g. epithelial cells, have such density that are still partially surrounded by feeder cells, that state may be evaluated by the microscope.

Examples of genetically modified cells that can be cultivated include, but are not limited to, cardiac cells, skeletal cells, mature skeletal muscle cells, smooth muscle cells, corneal epithelial cells, epithelial cells of the oral mucosa and epidermal cells. Preferably, said cells are corneal epithelial cells, epithelial cells of the oral mucous and epidermal cells, more preferably dermal cells. The cells can be derived from humans or animals. Cells can be genetically modified and then cultured directly from the source, like an animal, or can be cultured cells of a cell line stabilized or not.

Preferably the cells are cells derived from a biopsy and genetically modified in order to correct the low or absent expression of specific genes, in particular of genes involved in the EB, as beta-3 chain of laminin 5, collagen 7 or collagen 17.

Cell culture can be carried out by any method or under any condition provided that the culture is conducted on the surface of the fibrin-coated support.

Once subconfluence is reached, the culture medium is removed and the resulting cellular flap can be washed and detached from the support using, for example, pliers.

Any method known to the art expert for genetic modification of cells can be used in the present invention. In a preferred aspect of the invention, the genetically modified cells described herein are characterized by the fact that exogenous nucleic acid has been introduced by the use of a viral vector, for example in the form of a viral expression construct, more preferably a Retroviral vector.

Alternatively, the genetically modified cells described herein are characterized by the fact that exogenous nucleic acid is or comprises a construct of non-viral expression.

Preferably, in the vector as described above, the polynucleotide (or exogenous nucleic acid) is under the control of a promoter capable of expressing said polynucleotide efficiently.

The polynucleotide sequence in the vector is operatively linked to an appropriate expression control sequence (promoter) to direct the synthesis of the mRNA. Examples of promoters include the immediate promoter of early cytomegalovirus (CMV) genes, thymidine kinase HSV, early and late SV40, LTRs from retrovirus, preferably derived from murine leukemia virus (MLV). The vectors may also contain one or more selectable gene markers.

As used herein, the term “genetically modified cell” refers to a host cell that has been transduced, transformed or transfected with the polynucleotide or with the vector as described above.

As examples of suitable host cells, bacterial cells, fungal and yeast cells, insect cells, plant cells, animal cells, preferably human cells, and more preferably cells from biopsies of the skin, can be cited.

The introduction of the polynucleotide or vector previously described in the host cell may be carried out using methods known to the art expert, such as calcium phosphate transfection, DEAE-dextran mediated transfection, electroporation, lipofection, microinjection, viral infection, thermal shock, cell fusion, . . . .

The invention will be now illustrated by means of non-limiting examples referring to the following figures.

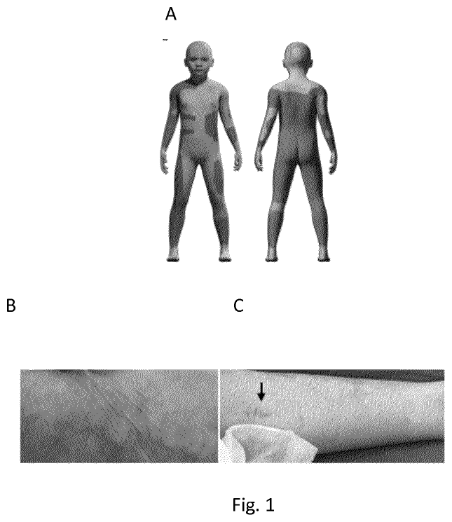

FIG. 1. Regeneration of the transgenic epidermis.

a, Schematic representation of the clinical picture. The denuded skin is indicated in red, while blistering areas are indicated in green. Flesh-colored areas indicate non-blistering skin. Transgenic grafts were applied on both red and green areas. Restoration of H's entire epidermis was obtained, with the exception of very few areas on the right thigh, buttocks, upper shoulders/neck and left axilla (altogether ≤2% of TBSA). b, Normal skin functionality and elasticity. c, Absence of blister formation at sites where some of post-graft biopsies were taken (arrow).

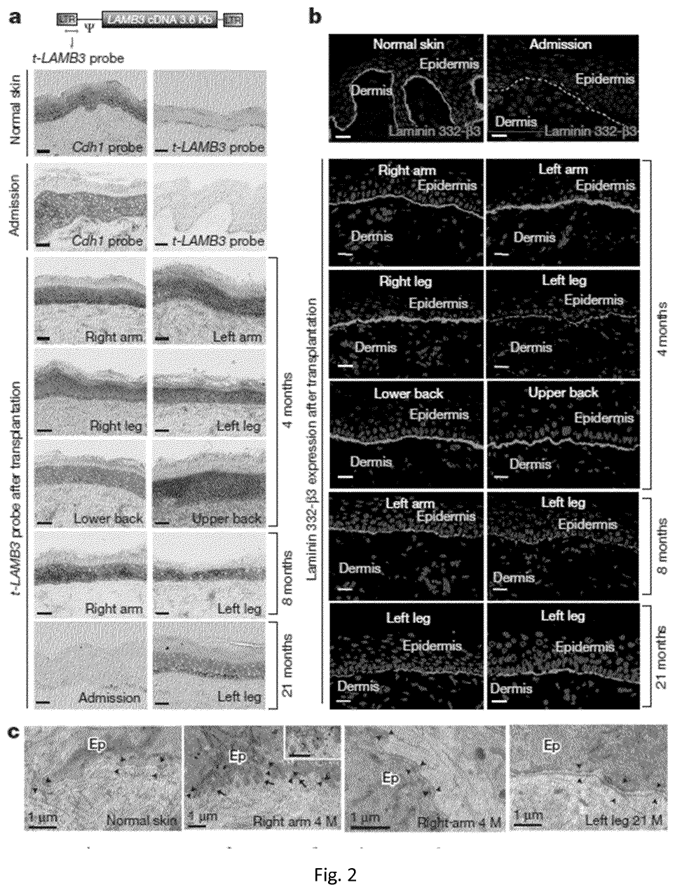

FIG. 2. Restoration of a normal epidermal-dermal junction.

Skin sections were prepared from normal skin, H′ affected (admission) and transgenic skin at 4, 8 and 21 months follow-up. a, In situ hybridization was performed using a transgene-specific probe (t-LAMB3) on 10-μm-thick sections. E-cadherin-specific probe (Cdh1) was used as a control. Scale bars, 40 μm. b, Immunofluorescence of laminin 332-β3 was performed with 6F12 moAbs on 7-μm-thick sections. DAPI (blue) marks nuclei. Dotted line marks the epidermal-dermal junction. Scale bars, 20 μm. c, Electron-microscopy was performed on 70-nm-thick skin sections. A regular basement membrane (arrows) and normal hemidesmosomes (arrowheads, higher magnification in the inset) are evident in H′ transgenic skin. Scale bars, 1 m.

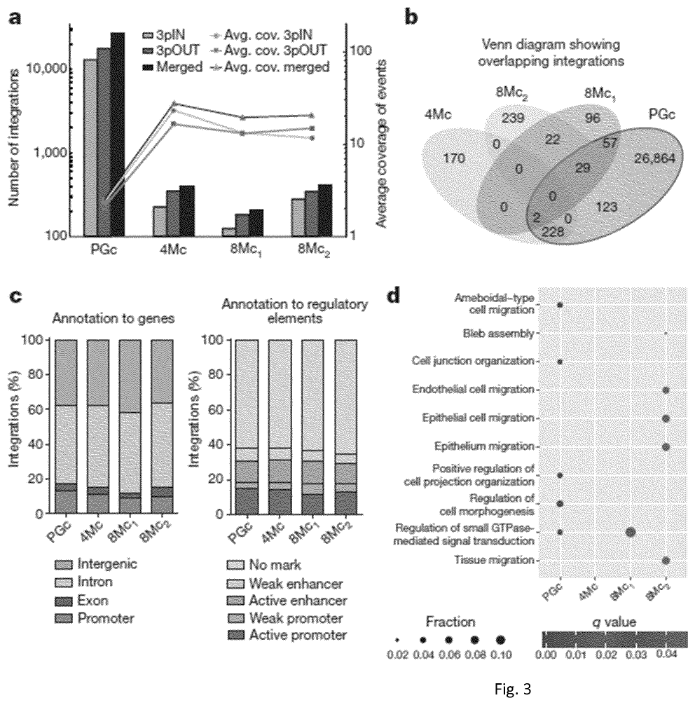

FIG. 3. Integration profile of transgenic epidermis.

a, Integrations were identified in libraries obtained using two LTR-primers (3pIN, light grey bars; 3pOUT, dark grey bars) and in the merged set (black bars). Lines (secondary axis) depict the average integration coverage, calculated after removal of PCR duplicates. b, Venn diagram of the number of shared integrations across samples. c, percentage of integrations mapped to: promoters, exons, introns, and intergenic regions (left); epigenetically defined active and weak promoters and enhancers, or genomic regions with no histone marks (right); (p-value>0.05; Pearson's Chi-squared test). d, Dot plot of the top 5 enriched GO Biological Process terms for each sample. Dot colour indicates statistical significance of the enrichment (q-value); dot size represents the fraction of genes annotated to each term.

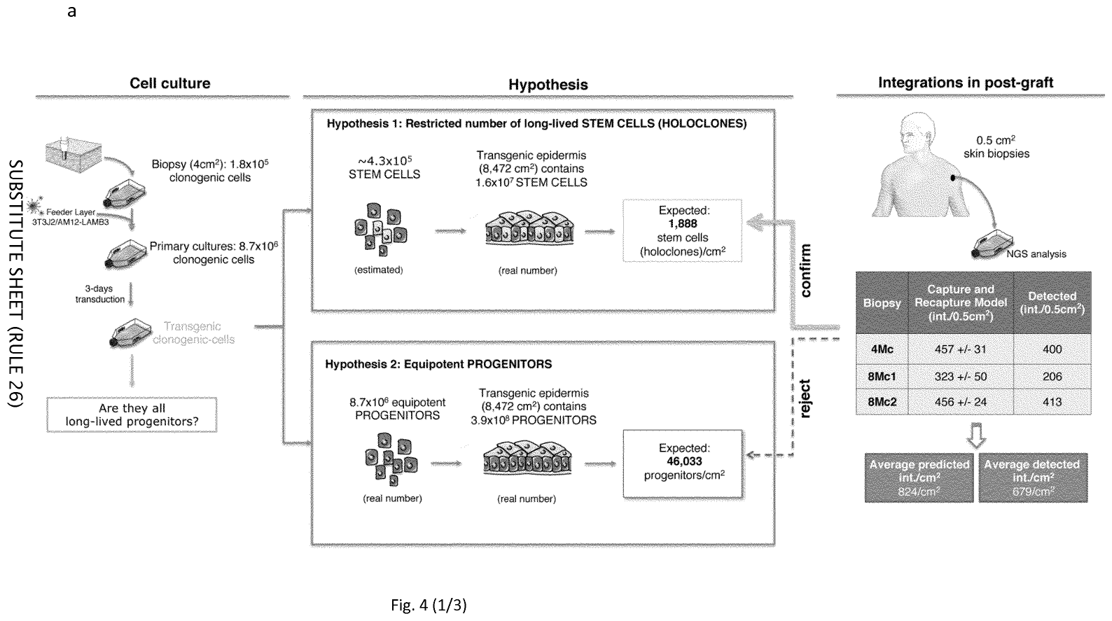

FIG. 4. Integration profile of stem and TA cells.

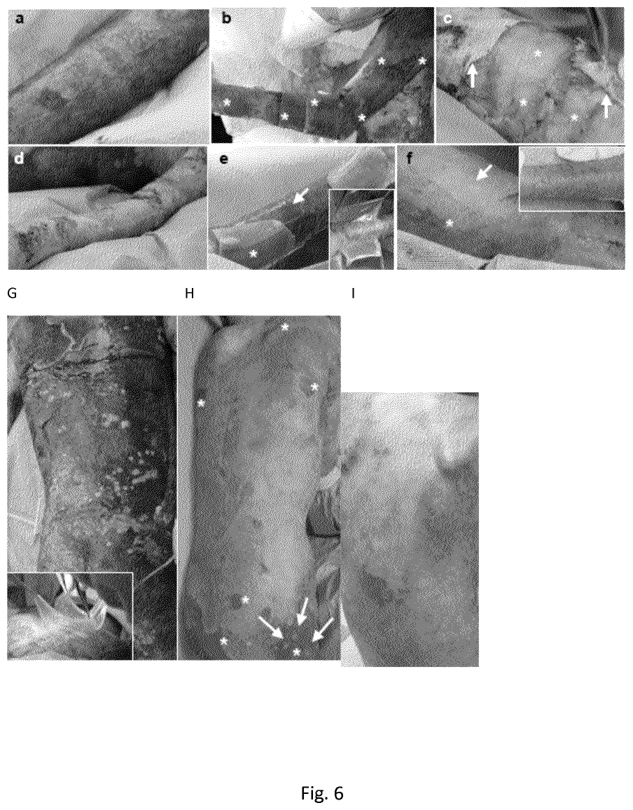

a, Clonogenic progenitors (blue cells) contained the original skin biopsy and in 8,742 cm² of transgenic epidermis are indicated. Stem cells, detected as holoclones (pink cells), were identified by clonal analysis (Methods and FIG. 9). The number of holoclones contained in the primary culture has been estimated. The schematic model posits the existence of specific long-lived stem cells generating pools of short-lived progenitors (Hypothesis 1) or a population of equipotent epidermal progenitors (Hypothesis 2). The number of integrations predicted by the Chapman-Wilson capture and re-capture model and formally detected by NGS analysis in 4Mc, 8Mc₁ and 8Mc₂ (right part of the panel) is consistent with the number of transplanted holoclones, hence fosters Hypothesis 1. b, Percentage of holoclone integrations recovered in the PGc bulk population. c, Holoclone integrations mapped to: promoters, exons, and introns, and intergenic regions (left); epigenetically defined active and weak promoters and enhancers, or genomic regions with no histone marks (right). d, The PGc pie chart (grey segment) shows that 91% of mero/paraclones did not contain the same integrations detected in the corresponding holoclones (each indicated by different blue segments). The 4Mc and 8Mc₁ pie charts (grey segments) show that such percentage decreased to 37% and 13%, respectively.

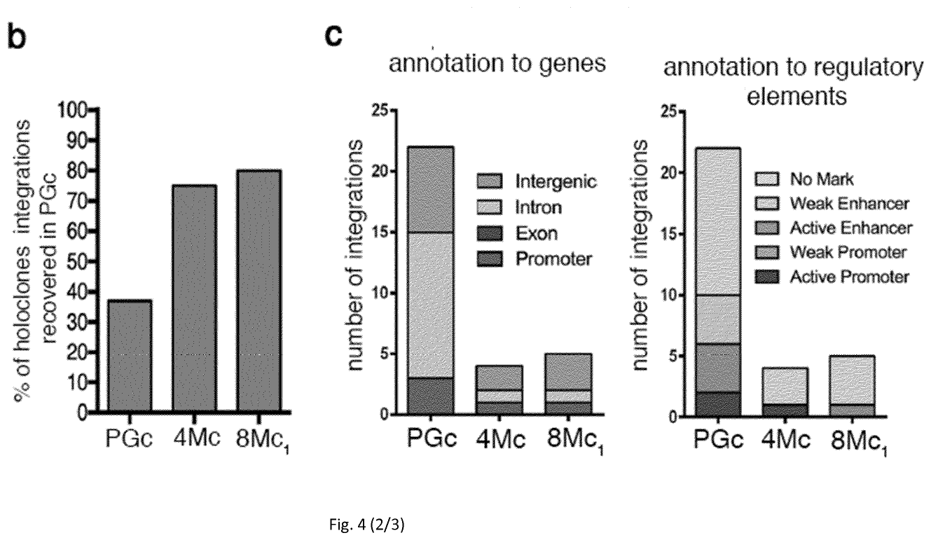

FIG. 5. Schematic representation of combined ex vivo cell and gene therapy.

The scheme shows the entire procedure, from skin biopsy to transplantation and follow up. Total number ofkeratinocytes, the corresponding clonogenic fraction and days of cultivation are shown for each passage. All analyses performed at each follow-up are indicated. Immunofluorescence (IF), in situ hybridization (ISH) and transmission electron microscopy (TEM) were performed on randomly taken 0.2-0.4 mm2 punch biopsies. Genome-wide analysis (NGS) was performed on Pre-Graft cultures (PGc) and on primary cultures initiated from ˜0.5 cm2 biopsies taken from the left leg (4Mc and 8Mc2) and the right arm (8Mc1). Clonal analysis and tracing were performed on PGc, 4Mc and 8Mc1

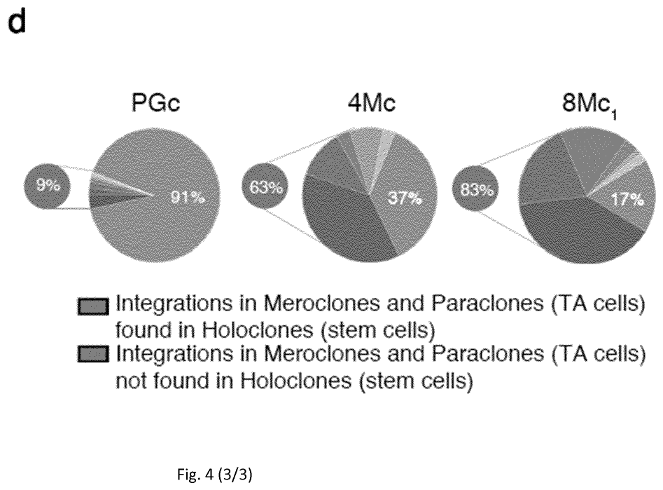

FIG. 6. Regeneration of the epidermis by transduced keratinocyte cultures.

a, Preparation of a dermal wound bed at the time of transplantation. b, Transplantation on the left arm of plastic-cultured epidermal grafts, mounted on a non-adhering gauze (asterisks). c, The engrafted epidermis (asterisks) is evident upon removal of the gauze (arrows), 10 days after grafting. d, Regenerated epidermis on the left arm at 1 month. e,f, Transplantation (e) and engraftment (f) of both plastic-cultured (asterisk) and fibrin-cultured (arrow and inset in e) grafts on the left leg. f (inset), Complete epidermal regeneration is evident at 1 month. g, The back of H was covered by fibrincultured grafts (inset). h, Complete epidermal regeneration was observed at 1 month, with the exception of some areas marked by the asterisks. Islands of epidermis were observed inside those denuded areas (arrows). i, Within 4 months, the regenerated epidermis surrounding the open lesions and the epidermal islands detected within those open lesions spread and covered the denuded areas.

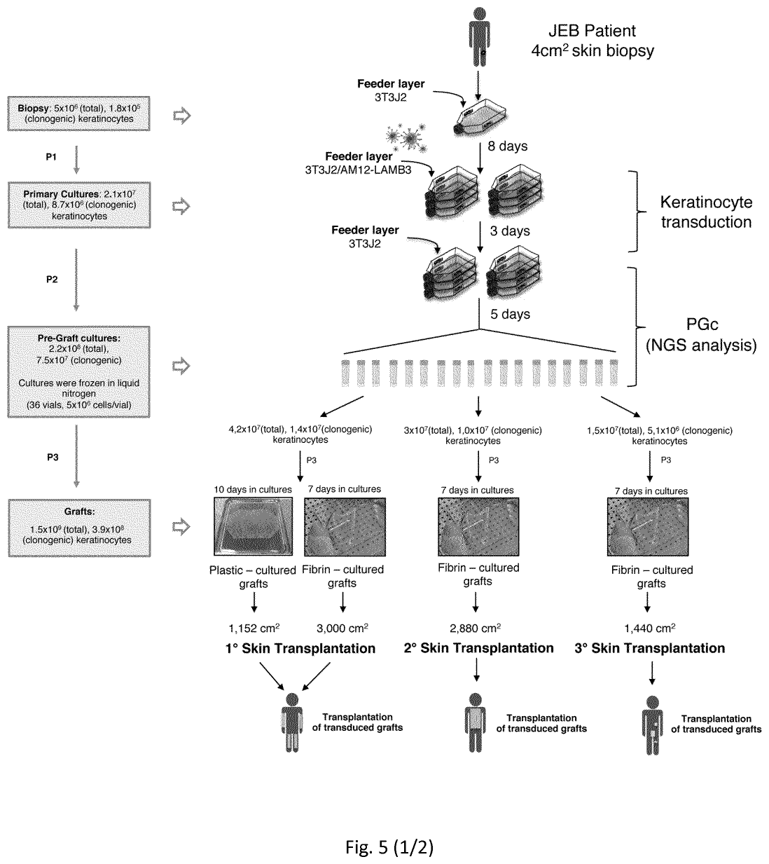

FIG. 7. Restoration of a normal dermal-epidermal junction.

a, Hematoxylin/Eosin staining of skin sections (7 μm thick) prepared from normal skin and from H at admission and at 4, 8 and 21-months follow-up. Black arrows show ruptures at the epidermal-dermal junction. Scale bar, 20 μm. b, Sections (7 μm thick) from normal skin, H's skin at admission and 21 months after transplantation were immunostained using laminin 332-α3, laminin 332-γ2, α6 integrin and β4 integrin antibodies. c, Adhesion of cohesive cultured epidermal sheets. Left panel: spontaneous detachment (arrows) of confluent laminin 332-β3 null H's keratinocyte cultures. Right panel: genetically corrected H's cultures remained firmly attached to the substrate. As with normal control cells, their detachment would require prolonged enzymatic treatment.

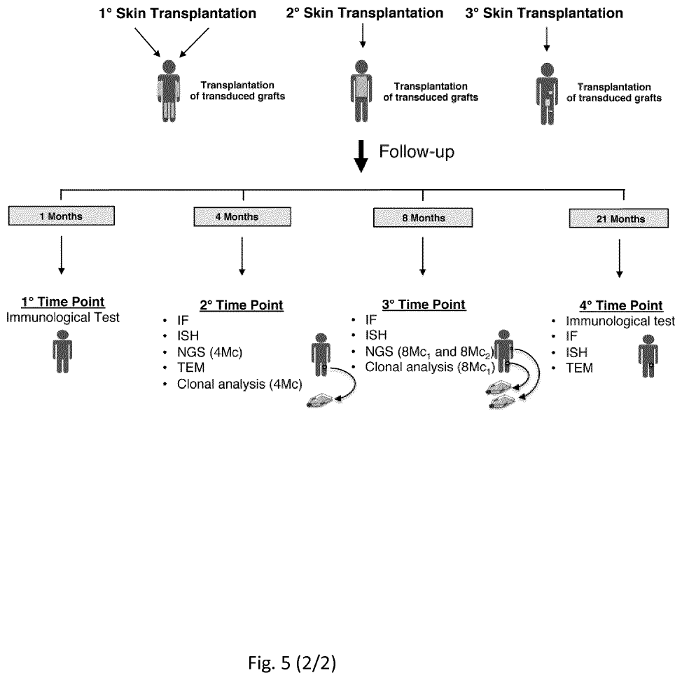

FIG. 8. Indirect immunofluorescence analysis.

To verify the absence of a humoral immune response to the transgene product, indirect immunofluorescence was performed by staining for antibasement membrane IgG auto-antibodies on monkey esophagus sections a, and normal human split skin (NH-SS) sections b, using H's plasma taken 21 months after transplantation. c, Positive control NH-SS sections (C+) were immunostained with an anti-human laminin-332 antibody (anti-GB3). Arrows denote the expected localization of the laminin 332 labelling. d and e, A healthy donor's plasma was used as negative control (C (−)) both in monkey esophagus (d) and normal skin sections (e). White arrows denote the expected localization of the laminin 332 labelling. To verify the absence of a humoral immune response to the transgene product, indirect immunofluorescence was performed by staining for antibasement membrane IgG auto-antibodies on monkey esophagus sections a, and normal human split skin (NH-SS) sections b, using H's plasma taken 21 months after transplantation. c, Positive control NH-SS sections (C+) were immunostained with an anti-human laminin-332 antibody (anti-GB3). Arrows denote the expected localization of the laminin 332 labelling. d and e, A healthy donor's plasma was used as negative control (C (−)) both in monkey esophagus (d) and normal skin sections (e). White arrows denote the expected localization of the laminin 332 labelling.

FIG. 9. Clonal analysis scheme

Sub-confluent cultures were trypsinized, serially diluted and inoculated (0.5 cell/well) onto 96-multiwell plates containing irradiated 3T3-J2 cells. After 7 d of cultivation, single clones were identified under an inverted microscope, trypsinized, transferred to 2 dishes and cultivated. One dish (¼ of the clone) was fixed 12 d later and stained with Rhodamine B for the classification of clonal type. The clonal type was determined by the percentage of aborted colonies formed by the progeny of the founding cell. The clone was scored as holoclone when 0-5% of colonies were terminal. When 95-100% of colonies were terminal (or when no colonies formed), the clone was classified as paraclone. When the amount of terminal colonies was between 5% and 95%, the clone was classified as meroclone. The second dish (¾ of the clone) was used for integration PGanalysis after 7 d of cultivation.

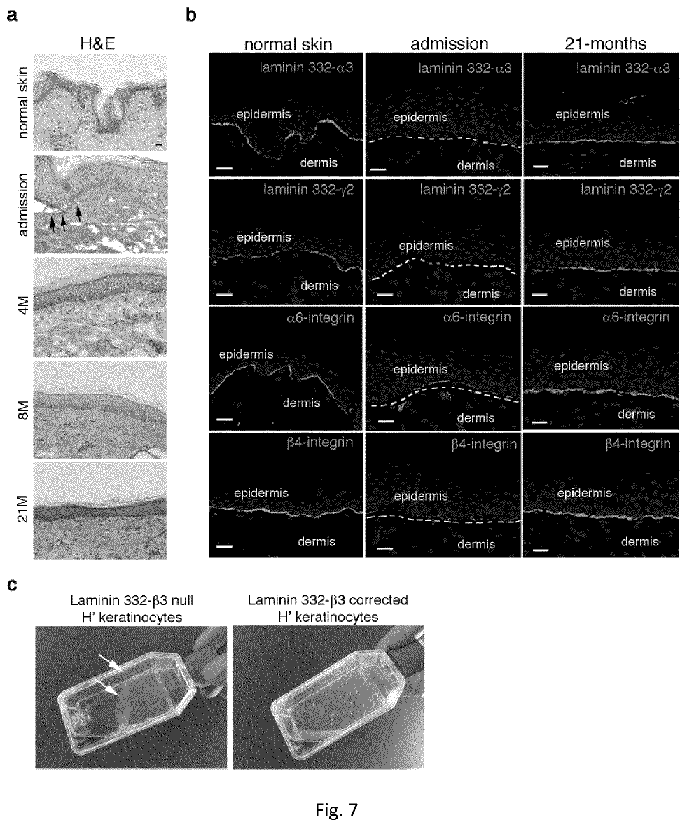

FIG. 10. Determination of provirus copy number.

Quantitative PCR (qPCR) was performed on genomic DNA of pre-graft cultures (PGc), primary cultures generated at 4 months (4Mc) and 8 months (8Mc1, 8Mc2) follow-up and selected holoclones (PRE.G_H1, PRE.G_H10, FU4m_H1-11, PRE.G_H7). All values are represented as the mean of 2 independent qPCR+SEM.

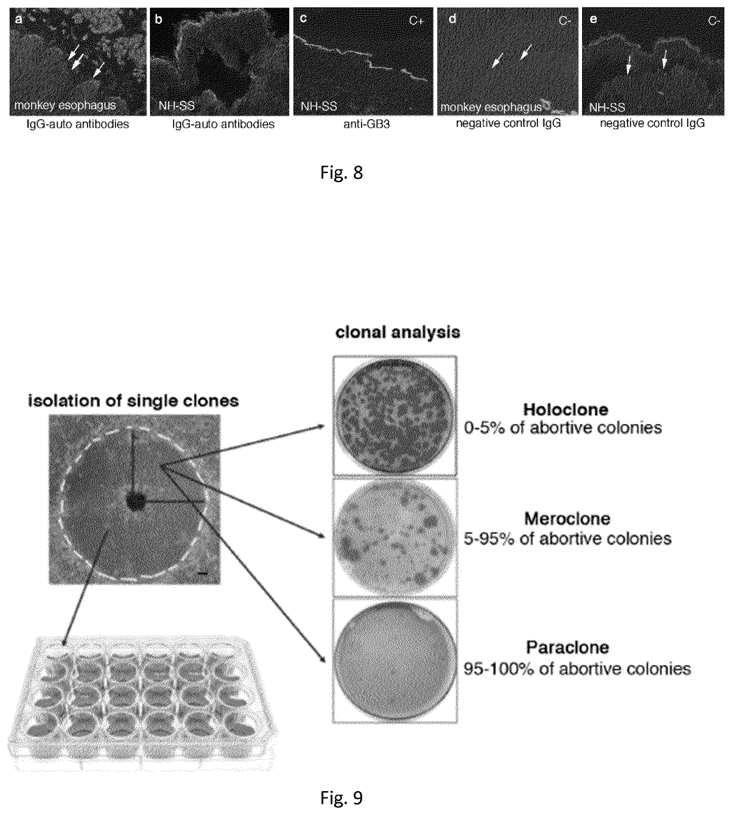

FIG. 11. Schematic model of holoclone tracing in the regenerated H's epidermis.

Transgenic epidermal cultures (PGc) contain of a mixed population of clonogenic basal stem cells (blue) and TA progenitors (grey). Upon engraftment and initial epidermal regeneration, both stem and TA cells can proliferate and eventually generate suprabasal terminally differentiated cells. Upon epidermal renewal (4 and 8 months), the short-lived TA progenitors (grey) are progressively lost. The long-lived stem cells then generate new pools of TA progenitors (now blue basal cells), which will produce terminally differentiated cells (suprabasal blue cells).

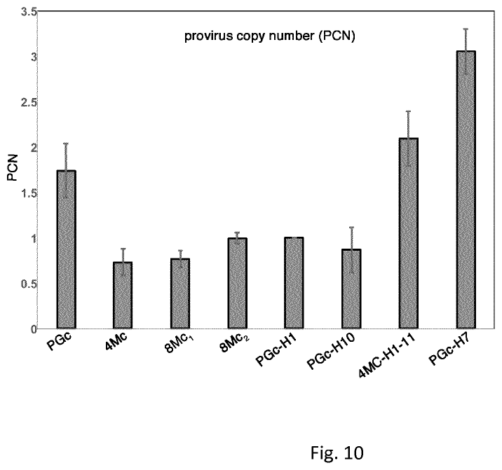

FIG. 12. Clinical Data

During hospitalization, H's inflammatory and nutritional status was documented by blood concentration of a, C-reactive protein (CRP) and b, albumin. The time course of biopsy sampling (marked by “B”) and epidermal culture transplantation is given by the arrows. The linear regressions visualize the trend of pre graft (dotted) and post graft (black line) progressions. The red line within the CRP time course demonstrates the CRP-limit, which is considered as a criterion for severe inflammation. These data demonstrate the critical situation ofH at admission and before transplantation and the improvement of his general status upon epidermal regeneration.

FIG. 13. Representative pictures of cultured keratinocytes grown on plastic. The image on the Right is representative of the flap prior to detachment and assembly for transport.

FIG. 14. Representative images of the flap detachment with Dispase II and two preparations of the flaps made from plastic. The center image shows a flap not conforming to the release due to the presence of air bubbles, while the photo on the right represents the image of a flap conforming to the release.

FIG. 15. Representative images of the confluences reached by growing keratinocytes on fibrin supports at the time of detachment and preparation for transport.

FIG. 16. Representative images of the preparation of the genetically modified epidermis flap.

### EXAMPLES

### Materials and Methods

### Patient, Clinical Course, Surgical, and Post-Operative Procedures.

Since birth, H repeatedly developed blisters, upon minor trauma, on the back, the limbs and the flanks, which occasionally caused chronic wounds persisting up to one year. Six weeks before the actual exacerbation, his condition deteriorated with the development of massive skin lesions. One day prior to admission, he developed fever followed by massive epidermal loss. He was admitted to a tertiary care hospital where topical wound care was performed using absorbable foam dressings (Mepilex, Mölnlycke Healthcare, Erkrath, Germany). As the patient appeared septic with elevated infection parameters, he initiated systemic antibiotic treatment with meropenem and vancomycin. Severe electrolyte imbalances required parenteral substitution of sodium, potassium, and magnesium. Swabs revealed Staphylococcus aureus and Pseudomonas aeruginosa. Due to the large wound area and further deterioration of his clinical condition, H was transferred to the paediatric burn centre of the Ruhr-University 4 days later. At admission, he suffered complete epidermal loss on ˜60% of total body surface area (TBSA), affecting all limbs, the back and the flanks. H was febrile, cachectic, with a total body weight of 17 kg (below 3^{rd} percentile), had signs of poor perfusion and C-reactive protein (CRP) was 150 mg/L. Antibiotic treatment was continued according to microbiologic assessment with flucloxacilline and ceftazidime. Retrospectively, the diagnosis of staphylococcal scalded skin syndrome was suspected due to flaky desquamations appearing 10 d after the symptoms began and Staphylococcus aureus was found on swabs. The iscorEB clinician score²⁹ was rated at 47. We initiated aggressive nutritional therapy by nasogastric tube (1100-1300 kcal/d) and additional parenteral nutrition (700 kcal/d kcal/kg/d, glucose 4 g/kg/d, amino acids 3 g/kg/d, fat 1.5 g/kg/d) according to his nutritional demands calculated using the Galveston formula. A necessary intake of about 1800 kcal/d was determined. Vitamins and trace elements were substituted as needed since zinc, selenium, and other trace elements were below the detection threshold. Beta-adrenergic blockade with propranolol was also started, as with severe burns³⁰. Due to bleeding during dressing changes and on-going loss of body fluids from the widespread skin erosions, the transfusion of 300 ml packed red blood cells was required every 7 to 12 days to keep the Hb value above 6-7 g/dl, and 20 g albumin were substituted once per week to keep albumin levels above 2.0 g/dl. Patient care was performed in accordance with the epidermolysis bullosa treatment guidelines³¹. H was bathed in povidone-iodine (PVP) solution or rinsed with polyhexanide-biguanide solution (PHMB) under general anaesthesia, first on a daily basis and subsequently every other day. We also employed several topical wound dressings and topic antimicrobials, including PHMB-gel and PVP ointment, without any significant impact on wound healing. However, wounds became cleaner and Staphylococcus aureus were no longer detectable for several weeks. H had persistent systemic inflammatory response syndrome (SIRS) with spiking fevers, wasting, and high values of acute-phase proteins (CRP, ferritin). He had chronic pain necessitating comprehensive drug management using fentanyl, dronabinol, gabapentin, amitryptiline and NSAIDs. Antibiotic treatment was continued according to swabs taken once weekly; swabs revealed intermittent wound infection with Pseudomonas aeruginosa and in the course Enterobacter cloacae, Enterococcusfaecalis and again Staphylococcus aureus. Treatment was changed biweekly omitting glycopeptides, carbapenemes and other drugs of last resort using mainly ceftazidime, cefepime, ampicilline, flucloxacilline, and tobramycin. Due to his life-threatening condition, we performed an unsuccessful allotransplantation of split-thickness skin grafts taken from his father. Despite an initial engraftment, complete graft loss occurred 14 days post-transplantation. Treatment attempts with Suprathel (Polymedics Innovation GmbH, Denkendorf, Germany), amnion, and glycerol preserved donor skin (Glyaderm, Euro Tissue Bank, Beverwijk, Netherlands) were unsuccessful as well. Further treatment attempts were judged to be futile by several experts in this field. After 5 weeks at the intensive care unit, H no longer tolerated nutrition via nasogastric or duodenal tube and began to vomit after small amounts of food. Due to massive hepatosplenomegaly, a PEG or PEJ was not feasible. A Broviac catheter was implanted and total parenteral nutrition was begun (1500 kcal/d, glucose 14 g/kg/d, amino acids 4 g/kg/d, fat 2 g/kg/d). Following an attempt of increased fat administration via parenteral nutrition, H developed a pancreatitis that resolved after omitting fat from the parenteral nutrition for a few days. With this nutritional regimen H's weight remained stable and blood glucose below 150 mg/dl was obtained without insulin administration. At this point, palliative care seemed the only remaining option. Because of the very poor short-term prognosis, we decided to start an experimental therapy approach using autologous epidermal stem cell-mediated combined ex-vivo cell and gene therapy (see Ethics Statement). Transgenic grafts were prepared, free of charge, under Good Manufacturing Practices (GMP) standards by Holostem Terapie Avanzate S.r.l. at the the Centre for Regenerative Medicine “Stefano Ferrari”, University of Modena and Reggio Emilia, Modena, Italy. On Oct. 19, 2015, we performed the first transplantation of transgenic cultures on the 4 limbs (and part of the flanks). At that time, H suffered complete epidermal loss on ˜80% of his body and still needed transfusion of 300 ml packed red blood cells every 7 to 12 days and 20 g albumin once per week to keep the albumin level above 2.0 g/dl. He continued suffering from spiking fevers, wasting, and high values for acute-phase proteins (CRP, Ferritin). Wounds were colonized with Staphylococcus aureus and Escherichia coli. Perioperative antibiotic therapy was performed with flucloxacilline, ceftazidime and ciprofloxacine. Under general anaesthesia, a careful and thorough disinfection with octenidine dihydrochloride (Schuelke & Mayr, Norderstedt, Germany) and surgical debridement of all limbs and flanks was performed, both with copper sponges and surgical knife. The debrided areas demonstrated a good perfusion with intact dermis. After achieving haemostasis using epinephrine soaked gauze, all debrided areas were washed thoroughly with saline to prevent epinephrine contact with cultured grafts. Grafts were carefully transplanted on the denuded, debrided areas and covered with Adaptic, a non-adhering dressing (Systagenix Wound Management, Gargrave, UK) and sterile dressing. Post-operatively, as total immobilization was recommended after the transplantation, H was maintained under continuous isoflurane sedation for 12 days using the AnaConDa system (SedanaMedical, Uppsala, Sweden). A catheter related blood-stream infection was successfully treated with vancomycin and meropeneme. Despite the use of clonidine and propofol, H developed a severe delirium after the isoflurane sedation, which was solved by levomepromazine. Engraftment was evaluated at 8-14 days. Epidermal regeneration was evaluated at 1 month (see text). Following the first transplantation, regular weekly transfusion of red blood cells and infusion of albumin was no longer necessary. The general condition improved and enteral nutrition became feasible again with the patient tolerating up to 400 kcal/d via nasogastric tube complementing the parenteral nutrition (1500 kcal/d, glucose 14 g/kg/d, amino acids 4 g/kg/d, fat 2 g/kg/d)³². On Nov. 23, 2015, a second transplantation was performed on the dorsum, the buttocks (and small areas on the shoulders and the left hand). These wounds were colonized with Staphyloccus epidermidis and Enterococcus faecium at the time of transplantation. Antibiotic treatment was done with vancomycin and ceftazidime due to suspected infection of the Broviac catheter. However, due to the high risk and severe side effects of long-term sedation, H was not sedated after the second transplantation. All dressings at the back and the buttocks had to be removed due to infection with Enterococcus faecium four days after transplantation. Topical antimicrobial therapy using polihexanide was started. On the dorsum, the graft healed in the following four weeks despite the early infection, and a stable skin without blister formation appeared (see text). Four weeks after the second transplantation, the CRP values remained below 100 mg/L and the patient was no longer febrile (FIG. 12). Complete enteral nutrition became feasible again. The affected body surface area remained below 10% TBSA. On January 2016, we performed a third procedure in a similar fashion covering the remaining defects on flanks, thorax, right thigh, right hand, and shoulders. These wounds were colonized with Staphylococcus epidermidis. The transplanted cells engrafted well. The patient could be withdrawn from his analgesics. The Broviac catheter was removed and the patient was discharged 7½ months after admission. At this time, he still had minor defects on the right thigh and the buttocks (FIG. 1 and FIG. 6). The iscorEB clinical score was 12. The transplanted skin was clinically stable and not forming blisters. The child returned back to regular elementary school on March 2016.

Cell lines. 3T3-J2 cells and Aml2-LAMB3 amphotropic packaging cells were grown as described below^{33,34}. A retroviral vector expressing the 3.6-kb full-length laminin 332 LAMB3 cDNA under the control of the MLV LTR was constructed in the MFG backbone³⁴ and integrated by transinfection in the amphotropic Gp+envAml2 packaging cell line³⁵ (additional details below). A master cell bank of a high-titre packaging clone Aml2-LAMB3 was made under GMP/GLP standards by a qualified contractor (Molmed S.p.A, Milan, Italy) according to the ICH guidelines.

### 3T3J2 Cell Line

Mouse 3T3-J2 cells were a gift from Prof. Howard Green, Harvard Medical School (Boston, Mass., USA). A clinical grade 3T3-J2 cell bank was established under GMP standards by a qualified contractor (EUFETS, GmbH, Idar-Oberstein, Germany), according to the ICH guidelines. GMP-certified 3T3-J2 cells have been authorized for clinical use by national and European regulatory authorities and cultured in Dulbecco's modified Eagle's medium (DMEM) supplemented with 10% irradiated calf serum, glutamine (4 mM) and penicillin-streptomycin (50 IU/ml).

### MFG-LAMB3-Packaging Cell Line

A retroviral vector expressing the full-length 3.6-kb LAMB3 cDNA under the control of the MLV LTR was constructed by cloning a 3.6-kb of LAMB3 cDNA (Gene Bank Accession # Q13751) into MFG-backbone¹³. A 5′ fragment of LAMB3 cDNA (563 bp) from the ATG to Stul site was obtained by PCR using as template the LB3SN plasmid³³. The PCR product was cloned into NcoI and BamHI sites of MFG-vector. The second fragment of LAMB3 cDNA (3050 bp) was obtained from LB3SN by enzyme digestion from Stul to XmnI and cloned into MGF-vector into Stul site. The entire cDNA of LAMB3 was fully sequenced. The Aml2-MGFLAMB3 producer cell lines were generated by transinfection in the amphotropic Gp+envAml2 packaging cell line³⁵. Briefly, plasmid DNA was introduced into the GP+E86 ecotropic packaging cell line³⁵ by standard calcium phosphate transfection. Forty-eighth ours after transfection, supernatant was harvested and used to infect the amphotropic packaging cell line GP+envAml2 ATCC n^{o} CRL 9641¹³ for 16 h in the presence of 8 ug/ml Polybrene. Infected Aml2 cells were clonally selected in HXM medium supplemented with 10% FCS, and containing 0.8 mg/ml G418 and 0.2 mg/mlhygromycin B (Sigma). Single colonies were screened for human LAMB3 production by immunofluorescence using an antibody specific for LAMB3 6F12 monoclonal antibody (from Dr. Patricia Rousselle, CNRS, Lyon) and for viral titer. The resulting producer cell lines showed a viral titer of 2×106 colony-forming units (cfu). A master cell bank of a high-titer packaging clone (Aml2-LAMB3 2/8) was made under GMP standards by a qualified contractor (Molmed S.p.A, Milan, Italy) according to the ICH guidelines and cultured in DMEM supplemented with 10% irradiated fetal bovine serum, glutamine (2 mM), and penicillin-streptomycin (50 IU/ml). All certifications, quality and safety tests (including detection on viruses and other micro-organisms both in vitro and in vivo) were performed under GMP standards for both cell lines.

Generation of Genetically Corrected Epidermal Sheets and Graft Preparation.

Primary cultures were initiated from a 4-cm² skin biopsy taken from a non-blistering area of inguinal region (informed consent was obtained). The entire cultivation and graft preparation procedures are detailed below. Briefly, sub confluent primary cells were plated (1 0.33×10⁴ cells/cm²) onto a feeder-layer (8×10⁴ cells/cm²) composed of lethally irradiated 3T3-J2 cells and producer GP+envAml2-LAMB3 cells³⁶ (a 1:2 mixture) in keratinocytes growth medium (KGM)³³. Sub-confluent transduced cultures were pooled, re-suspended in KGM supplemented with 10% glycerol, aliquoted, and frozen in liquid nitrogen (36 vials, 5.1×10⁶ cells/vial). At each step, efficiency of colony formation (CFE) by keratinocytes was determined, fixing colonies with 3.7% formaldehyde 12 days later and staining them with 1% Rhodamine B³⁶.

For the preparation of plastic-cultured grafts, transduced keratinocytes were thawed and plated (1×10⁴ cells/cm²) on 100 mm culture dishes containing lethally irradiated 3T3-J2 cells and grown to confluence in KGM with no penicillin-streptomycin. Grafts were then detached with Dispase II, 2.5 mg/ml (Roche Diagnostics S.p.a.) and mounted basal side up on sterile non-adhering gauze (Adaptic, Systagenix Wound Management, Gargrave, UK). For fibrin-cultured grafts, fibrin gels were prepared in 144 cm² plates (Greiner, Stuttgart, Germany) as described³⁶⁻³⁸. Fibrin gels consisted of fibrinogen (23.1 mg/ml) and thrombin (3.1 IU/ml) in NaCl (1%), CaCl₂ (1 mM) and Aprotinin (1786 KIU/ml).

Fibrin is produced by the inventor consists of two fibrinogen reagents (23.1 mg/ml) and thrombin (3.1 IU/ml) produced by Kedrion (commercial name Kolfib). The production process preferably involves three phases:

1. Preparation of fibrinogen solution and thrombin

2. Preparation of fibrin support

3. Fibrin compliance test

1. A thrombin (kedrion) vial containing 625 IU or 1250 IU of thrombin is reconstituted in 10 ml of buffer consisting of NaCl (1.1%) and CaCl₂ (1 mM). The entire content is then transferred to a 50 ML tube to which other 10ML buffers will be added. If the starting vial contained 625 UI of thrombin, a 1:5 dilution of the reconstituted solution was made. If the starting vial contained 1250 UI of thrombin, a dilution of 1:10 of the reconstituted solution was made. The solution is prepared at room temperature and examined to ensure that there are no solubilized thrombin solutions. A 120 mg or 240 mg fibrinogen was solubilized in 2.59 ML or 5.184 ML buffer containing NaCl (1%) and CaCl₂ (1 mM) and aprotinin (1786 KIU/ml). The reconstituted solution is incubated at 36.5° C. for 30 to 60 minutes to complete the solubilization.

2. The fibrin gel is prepared in a 144 cm² support in untreated plates for cell culture. To obtain a 100 mm thick gel, 6 ML of thrombin solution and 6 ML of fibrinogen solution are mixed to obtain a homogeneous mixture. The plates thus prepared are left at room temperature for 10-15 min until full polymerization and then stored at 4° C. for up to one month.

3. Before releasing the fibrin gel are subjected to compliance checks.

Transduced keratinocytes were thawed and plated (1×10⁴ cells/cm²) on lethally irradiated 3T3-J2 cells onto the fibrin gels and grown as above. Grafts were washed twice in DMEM containing 4 mM glutamine, and placed in sterile, biocompatible, non-gas-permeable polyethylene boxes containing DMEM and 4 mM glutamine. Boxes were closed, thermo-sealed and packaged into a sealed, sterile transparent plastic bag for transportation to the hospital.

Cell Culture and Medium.

Transgenic cultured epidermal grafts were prepared under GMP standards by Holostem Terapie Avanzate S.r.l. at the Centre for Regenerative Medicine “Stefano Ferrari”, University of Modena and Reggio Emilia, Modena, Italy. Briefly, a 4-cm2 skin biopsy was minced and trypsinized (0.05% trypsin and 0.01% EDTA) at 37° C. for 3 h. Cells were collected every 30 min, plated (2.7×104 cells/cm2) on lethally irradiated 3T3-J2 cells (2.66×104 cells/cm2) and cultured in 5% CO2 and humidified atmosphere in keratinocyte growth medium (KGM):DMEM and Ham's F12 media (2:1 mixture) containing irradiated fetal bovine serum (10%), insulin (5 μg/ml), adenine (0.18 mM), hydrocortisone (0.4 μg/ml), cholera toxin (0.1 nM), triiodothyronine (2 nM), glutamine (4 mM), epidermal growth factor (10 ng/ml), and penicillin-streptomycin (50 IU/ml). Sub-confluent primary cultures were trypsinized (0.05% trypsin and 0.01% EDTA) at 37° C. for 15-20 minutes and seeded (1.33×104 cells/cm²) onto a feeder-layer (8×104 cells/cm²) composed of lethally irradiated 3T3-J2 cells and producer GP+envAml2-LAMB3 cells¹² (a 1:2 mixture) in KGM. After 3 days of cultivation, cells were collected and cultured in KGM onto a regular 3T3-J2 feeder-layer. Sub-confluent transduced cultures were pooled, re-suspended in KGM supplemented with 10% glycerol, aliquoted, and frozen in liquid nitrogen (36 vials, 5×106 cells/vial). At each step, efficiency of colony formation (CFE) by keratinocytes was determined by plating 1000 cells, fixing colonies with 3.7% formaldehyde 12 days later and staining them with 1% Rhodamine B.

Clonal Analysis and DNA Analysis.

Clonal analysis was performed as described⁷ and shown in FIG. 9. Sub-confluent epidermal cultures were trypsinized, serially diluted and plated in 96 wells plates (0.5 cells/well). After 7 d of cultivation, single clones were identified under an inverted microscope and trypsinized. A quarter of the clone was cultured for 12 days onto a 100 mm (indicator) dish, which was then fixed and stained with Rhodamine B for the classification of clonal type³⁹. The remaining part of the clone (¾) was cultivated on 24-multiwell plates for genomic DNA extraction and further analysis (FIG. 9).

Library Preparation and Sequencing.

Illumina barcoded libraries were obtained from 3 independent pre-graft cultures (PGc, generated by 3 vials, each containing ˜220,000 clonogenic keratinocytes) and 3 post-graft cultures (4Mc, 8Mc₁, and 8Mc₂). For each sample, 2 tubes with 500 ng of genomic DNA were sheared in 100 μl of water applying 3 sonication cycles of 15 sec/each in a Bioruptor (Diagenode) to obtain fragments of 300-500 bp. Fragmented DNA was recovered through purification with 0.8 volumes of Agencourt AMPure XP beads, two washing steps with 80% ethanol, and elution in Tris-HCl 10 mM. Repair of DNA ends and A-tailing of blunt ends were both performed using Agilent SureSelectx^{T} reagents (Agilent Technologies), according to manual specifications, followed by purification with 1.2 volumes of AMPure XP beads. A custom universal adapter was generated by annealing <Phos-TAGTCCCTTAAGCGGAG-C3> (SEQ ID NO:11) oligo and <GTAATACGACTCACTATAGGGCNNNNNNCTCCGCTTAAGGGACTAT> (SEQ ID NO:12) oligo on a thermocycler from 95° C. to 21° C., with decrease of 1° C./min in a 10 mM Tris-HCl, 50 mM NaCl buffer. Ligation of universal adapter to A-tailed DNA was carried out in a reaction volume of 30 μl with 400 U of T4 DNA ligase (New England Biolabs) with respective T4 DNA ligase buffer 1× and 35 pmol of dsDNA universal adapter and incubated at 23° C. for 1 h, at 20° C. for 1 h, and finally heat inactivated at 65° C. for 20 min. Each ligation product was purified with 1.2 volumes of AMPure XP beads as described above. Eluate of each reaction was split in 3 different tubes to perform independent PCR reaction in order to mitigate reaction-specific complexity reduction. Each tube was amplified by PCR with a combination of I7-index primers (701/702/703), to multiplex samples on the same Illumina sequencing lane, and of two 15 LTR-primers (501/502) to barcode specific enrichments of MLV-LTR sequences (Table 7).

TABLE 7
List of I7-index primers and
I5 LTR-primers used for library preparation.

| Primer | | |
|---|---|---|
| set | Primer name | Primer sequence |
| I7 | Linker_primer_701_N | CAAGCAGAAGACGGCATACGAGATCGAGTA |
| | | ATGTGACTGGAGTTCAGACGTGTGCTCTTC |
| | | CGATCTGTAATACGACTCACTATAGGGC |
| | | (SEQ ID NO: 13) |
| | Linker_primer_702_N | CAAGCAGAAGACGGCATACGAGATTCTCCG |
| | | GAGTGACTGGAGTTCAGACGTGTGCTCTTC |
| | | CGATCTGTAATACGACTCACTATAGGGC |
| | | (SEQ ID NO: 14) |
| | Linker_primer_703_N | CAAGCAGAAGACGGCATACGAGATAATGAG |
| | | CGGTGACTGGAGTTCAGACGTGTGCTCTTC |
| | | CGATCTGTAATACGACTCACTATAGGGC |
| | | (SEQ ID NO: 15) |
| I5 | MuLV_LTR-3pIN_501_N | AATGATACGGCGACCACCGAGATCTACACT |
| | | ATAGCCTACACTCTTTCCCTACACGACGCT |
| | | CTTCCGATCTGACTTGTGGTCTCGCTGTTC |
| | | CTTGG |
| | | (SEQ ID NO: 16) |
| | MuLV_LTR-3pOUT_502_N | AATGATACGGCGACCACCGAGATCTACACA |
| | | TAGAGGCACACTCTTTCCCTACACGACGCT |
| | | CTTCCGATCTGGGTCTCCTCTGAGTGATTG |
| | | ACTACC |
| | | (SEQ ID NO: 17) |

PCR reaction was carried out in a final volume of 25 μL, with 20 pmoles of each primer and Phusion High-Fidelity master mix 1× (New England Biolabs). PCR products were purified with 0.8 AMPure XP beads and all amplification products from the same sample (2 fragmentations, 3 PCR reactions) were pooled and quantified on Bioanalyzer 2100 high sensitivity chip. Paired-end 125 bp sequencing was performed on Illumina HiSeq2500 (V4 chemistry). Illumina barcodes on the whole Illumina lanes were combined to maintain a minimum hamming-distance of at least 3 nucleotides. Extraction and de-multiplexing of reads was obtained using CASAVA software (v. 1.8.2) applying a maximum barcode mismatch of 1 nucleotide and considering the dual indexing of 17-15 sequences. Reads were processed using the bioinformatics pipeline described in details below. Briefly, reads were first inspected with cutadapt⁴⁰ to verify specific enrichments, then trimmed using FASTX-Toolkit (http://hannonlab.cshl.edu/fastx_toolkit/) and bbduk2 (http://jgi.doe.gov/data-and-tools/bbtools/) to remove adaptors and primers, and mapped to the human genome reference sequence GRCh37/hgl9 using BWA MEM⁴¹ with default parameters and the −M flag. Finally, the start coordinate of the alignment was used as the putative integration site.

### Bioinformatics Analysis of Sequencing Data.

To process the sequencing reads we assembled a custom bioinformatics pipeline composed of standard tools for NGS data analysis. In particular, we first used cutadapt (v1.14; https://cutadapt.readthedocs.io/en/stable/)⁴⁰ to verify the presence, in read pairs, of specific sequences indicative of a successful enrichment. Specifically, in the read harboring the 15 LTR-primer sequence (read 1), we searched for the primer sequence and, at its 3′-end, for the remainder LTR sequence. Instead, in the read harboring the 17 indexing primer (read 2), we searched for the presence of the common adapter sequence preceding the 6 indexing bases. Pairs containing both sequences were retained for analysis after trimming the 15 primer and the remainder LTR sequence in read 1 and the common adapter sequence in read 2. Then, we used FASTX-Toolkit (http://hannonlab.cshl.edu/fastx_toolkit/) to remove from read 2 the first 6 indexing bases, utilized as de-duplicator component during de-multiplexing. Since half of the amplification products are expected to be non-informative in the detection of the insertion site, given the identity of the two LTRs of the MLV genome, we applied bbduk2 (http://jgi.doe.gov/data-and-tools/bbtools/) to identify and remove read pairs representing inward-facing LTR primer enrichment events. In bbduk2 we set the kmer length to 27 (k=27) and the edit distance and the maxbadkmers parameters both to 1. Reads were aligned on the human genome reference sequence GRCh37/hgl9 using BWA MEM⁴¹ with default parameters and the −M flag (to include multiple-mapping signature in the BAM file). Read pairs sharing the same mapping coordinates and the same de-duplicator component were labeled as PCR duplicates and removed. Aligned read pairs were further filtered to retain only those mapping at a distance comprised between 150 and 600 bp (corresponding to the expected library insert size), allowing a maximum of 1 bp soft-clip (unaligned) on all ends, with the exception of the 5′ end of read 2 where we allowed 20 bp soft clip since it contains the 18 bp untrimmed common adapter sequence. Finally, we retained read 1 sequences with a minimum mapping quality of 40 and extracted and counted the alignment coordinates of their first base, representing the putative insertion site. Insertion sites within 10 bp from one another were treated as a single insertion, their counts summed using BEDTools (v2.15; http://bedtools.readthedocs.io/en/latest/content/bedtools-suite.html)⁴², and the summed count assigned to left coordinate. When intersecting insertion sites across samples, we considered overlapping those insertion events closer than 30 bp.

Genomic and Functional Annotation of Integration Events.

Annotation of integration sites to gene features was performed using the ChlPseeker R package⁴⁰. Insertion sites were mapped to promoters (defined as 5 kb regions upstream of the transcription start site), exons, and introns of RefSeq genes, and intergenic regions. Functional enrichment in GO Biological Processes of genes harboring an integration site was performed using the clusterProfiler R package⁴⁰, setting a q-value threshold of 0.05 for statistical significance. Annotation of integration sites to epigenetically defined transcriptional regulatory elements was performed with the BEDTools suite⁴² using publicly available ChIP-seq data of histone modifications (H3K4me3, H3K4mel, and H3K27ac) in human keratinocyte progenitors (GSE64328)⁴⁰.

Linear Amplification-Mediated (LAM) PCR, NGS on Holoclones, PCR on Mero/Paraclones and Integration Site Analysis.

100 ng of DNA of transduced keratinocytes was used as template for LAM-PCR. LAM-PCR product was initiated with a 50-cycle linear PCR and digested with 2 enzymes simultaneously without splitting the DNA amount using 1 μl MseI (5 U/μl) and 1 μl PstI (5U/μl) (Thermo Fisher, Waltham, US) and ligation of a MseI restriction site-complementary linker cassette. LAM-PCR was digested with 2 enzymes simultaneously without splitting the DNA amount. The second enzyme PstI was introduced to eliminate the undesired 5′LTR-LAMB3 sequences. The first exponential biotinylated PCR product was captured via magnetic beads and reamplified by a nested second PCR. LAM-PCR primers for MLV-LAMB3 used are in table 8. For the initial LAM-PCR, the 5′-biotinylated oligonucleotide complementary to the 3′-LTR sequence (5′-GGTACCCGTGTATCCAATAA-3′) (SEQ ID NO:18) was used for the linear amplification step. The 2 sequential exponential amplification steps were performed with nested oligonucleotides complementary to the 3′-LTR sequence (5′-GACTTGTGGTCTCGCTGTTCCTTGG-3′) (SEQ ID NO:19); (5′-GGTCTCCTCTGAGTGATTGACTACC-3′) (SEQ ID NO:20), each coupled with the oligonucleotides complementary to the linker cassette (Table 8).

TABLE 8
List of primers used for LAM-PCR on holoclones.

| Primer name | Primer sequence |
|---|---|
| MLV 3′LTRIin_biotin | GGTACCCGTGTATCCAATAA |
| | (SEQ ID NO: 21) |
| MLV 3′LTR_biotin | GACTTGTGGTCTCGCTGTTCCTTGG |
| | (SEQ ID NO: 22) |
| LCrv | GTAATACGACTCACTATAGGGC |
| | (SEQ ID NO: 23) |
| MLV 3′LTR nested | GGTCTCCTCTGAGTGATTGACTACC |
| | (SEQ ID NO: 24) |
| LCrv | AGGGCTCCGCTTAAGGGAC |
| | (SEQ ID NO: 25) |
| LC1 TAlinkerMse(+) | GTAATACGACTCACTATAGGGCTCC |
| | GCTTAAGGGAC |
| | (SEQ ID NO: 26) |
| LC2 TAlinkerMse(−) | TAGTCCCTTAAGCGGAG |
| | (SEQ ID NO: 27) |

LAM-PCR amplicons were either separated on 2% standard agarose gels (Biozym, Hessisch Oldendorf, Germany) and the excised bands cloned into the StrataClone PCR Cloning Kit (Agilent Technologies, Santa Clara), PCR-purified using High Pure PCR Product Purification Kit (Roche, Basel, Switzerland), shotgun cloned, and sequenced by Sanger, or used as unpurified PCR product as template for NGS library preparation. The fragments were end-repaired, adaptor-ligated, nick-repaired and purified by using the Ion Plus Fragment Library Kit (Life Technologies, Carlsbad, US). The template preparation and the sequencing run on the machine were also performed according to the protocols of Life Technologies. A mean vertical coverage was planned to reach at least 2000 reads. Data were analyzed as described below.

Screening of the integration sites of the meroclones and paraclones was done by PCR using a combination of the FW primer MLV 3′LTR control F (5′-GGACCTGAAATGACCCTGTG-3′) (SEQ ID NO:28) of the LTR and a specific reverse primer (Table 9)

TABLE 9
List of primers used for PCR on
meroclones and paraclones in PGc, 4Mc, and 8Mc₁.

| Culture | Primer name | Primer sequence |
|---|---|---|
| PGc | MLV 3′LTR | GGACCTGAAATGACCCTGTG |
| | control F | (SEQ ID NO: 29) |
| | Chr.5a | ACCCACAGCTCCTGTCTCAT |
| | | (SEQ ID NO: 30) |
| | Chr.2a | TTCTTTCAGTCTGGTGGGGTG |
| | | (SEQ ID NO: 31) |
| | Chr.4a | TGGTGGTGGAGTATCTGGAG |
| | | (SEQ ID NO: 32) |
| | Chr.4b | GTGGTGGTGGAGTATCTGGAG |
| | | (SEQ ID NO: 33) |
| | Chr.19a | CTCACCATCATGAGGAGCAA |
| | | (SEQ ID NO: 34) |
| | Chr.19b | CTCACCATCATGAGGAGCAA |
| | | (SEQ ID NO: 35) |
| | Chr.5b | GAGCAATTTGAGGGTCAGAGA |
| | | (SEQ ID NO: 36) |
| | Chr.17c | GAAATCAAGATTGTATCACGTTCC |
| | | (SEQ ID NO: 37) |
| | Chr.16 | CTGCACACATGCCCTCTTT |
| | | (SEQ ID NO: 38) |
| | Chr.2b | TCCCAGGAACTTTGTTCAGA |
| | | (SEQ ID NO: 39) |
| | Chr.3 | CCCTAAGGAGCTCCAACTGA |
| | | (SEQ ID NO: 40) |
| | Chr.Y | CTGAGGATGGTGGCAGAAAT |
| | | (SEQ ID NO: 41) |
| | Chr.6 | GCCAATTAACACTCGTTCACC |
| | | (SEQ ID NO: 42) |
| | Chr.14b | GGCTCCCAGGTATGTTCTCA |
| | | (SEQ ID NO: 43) |
| 4Mc | Chr.1 | CCTGATGTTCTGTCCCCCTA |
| | | (SEQ ID NO: 44) |
| | Chr.9a | GCATGCACAACAGCTCAAAC |
| | | (SEQ ID NO: 45) |
| | Chr.14a | GCCTCCATTTGGAGAGAAAAT |
| | | (SEQ ID NO: 46) |
| | Chr.15a | CCTCCTCCTCTTCCCTTGAT |
| | | (SEQ ID NO: 47) |
| 8Mc₁ | Chr.8 | CGGCAACCACTTTAAAGGAC |
| | | (SEQ ID NO: 48) |
| | Chr.9b | GCCTCACTTTCTTTCTCTGTAAATG |
| | | (SEQ ID NO: 49) |
| | Chr.17a | GGCTCACTGCAACCTTCATC |
| | | (SEQ ID NO: 50) |
| | Chr.X | CTGGAGCTGGGTGAGATAAAG |
| | | (SEQ ID NO: 51) |
| | Chr.5c | GGAATGGGGCATAAGAGACA |
| | | (SEQ ID NO: 52) |
| | Chr.17d | TTGAGATAGTCTTACGCTGTCACC |
| | | (SEQ ID NO: 53) |

in the proximity of the integration site. Genomic DNA from the holoclones was used as positive controls.

Calculation of the Expected Number of Integrations.

The expected number of integrations (i.e., the expected population size) in PGc, 4Mc, 8Mc1, and 8Mc2 samples was calculated in R applying a capture-recapture model based on the Chapman's estimate and its confidence intervals¹⁵ (Chapman, D. G. & University of California, B. Some properties of the hypergeometric distribution with applications to zoological sample censuses. (University of California Press, 1951)).

$$\hat{N} = \frac{( {n}_{1} + 1 ) ( {n}_{2} + 1 )}{{n}_{11} + 1} - 1$$

$$\hat{N} \pm {Z}_{1 - α / 2} \sqrt{\frac{( {n}_{1} + 1 ) ( {n}_{2} + 1 ) {n}_{21} {n}_{12}}{{( {n}_{11} + 1 )}^{2} ( {n}_{11} + 2 )} \vert}$$

where

\hat{N}

is the estimated number of integrations, n₁ is the number of integrations found in the 3pIN library, n₂ those found in the 3pOUT library, n₁₁ the number of overlapping integrations, n₁₂ and n₂₁ the insertion respectively exclusive of 3pIN and 3pOUT, respectively, and Z_{1-α/2}=2.56

for α=0.01.

Provirus Copy Number (PCN)

TaqMan PCR analysis was performed with TaqMan Universal PCR Master Mix and vector-specific LAMB3 and GAPDH probes (LAMB3: Hs00165078_m1; GAPDH: Hs03929097_g1, Applied Biosystems). The amplicon for LAMB3 was located between adjacent exons to recognize only provirus LAMB3. Reactions were performed with ABI Prism 7900 Sequence Detection System (Applied Biosystems), using 10 ng of genomic DNA. The relative quantity that relates the PCR signal of the target provirus was normalized to the level of GAPDH (internal control gene) in the same genomic DNA by using the 2^{-ΔΔCT} quantification.

Immunofluorescence (IF), Transmission Electron Microscopy (TEM) and Hematoxylin/Eosin Staining.

These procedures are detailed below. H's skin biopsies were taken after the parent's informed consent at 4, 8, and 21 months follow-up. The following antibodies were used for IF: mouse 6F12 monoclonal antibody to laminin 332-β3, laminin 332-α3 BM165 mAb (both from Dr. Patricia Rousselle, CNRS, Lyon), laminin 332-γ2 D4B5 mAb (Chemicon), α6 integrin 450-30A mAb and β4 integrin 450-9D mAb (Thermo Fisher Scientific). Alexa Fluor 488 goat anti-mouse (Life Technologies) conjugated secondary antibodies were used for detection. Cell nuclei were stained with DAPI. The following vector-specific primers were used for ISH: 5′-Sp6-AGTAACGCCATTTTGCAAGG-3′ (Tm 60° C.) (SEQ ID NO:54) and 5′-T7-AACAGAAGCGAGAAGCGAAC-3′ (SEQ ID NO:55) (Tm 58° C.)^{36,43}

### Immunofluorescence on Skin Section and Cells.

Normal skin biopsies were obtained as anonymized surgical waste, typically from abdominoplasties or mammoplasty reduction and used as normal control. Ethical approval for obtaining the tissue, patient information sheets, and consent forms have been obtained and approved by our institutions (Comitato Etico Provinciale, Prot. N^{o} 2894/C.E.). H's skin biopsies were taken randomly from his body upon agreement patient information sheets and consent forms. Skin biopsies were washed in PBS, embedded in Killik-OCT (Bio-Optica) and frozen. Immunofluorescence was performed on 7 m skin sections (fixed in PFA 3%, permeabilized with PBS/triton 0.2% for 15 min at r.t. and blocked 1 h at r.t with BSA 2% in PBS/triton 0.2%) using antibodies (described into methods section) in BSA 2% in PBS/triton 0.2% and added to skin sections for 30 min at 37° C. Sections were washed 3 times in PBS/triton 0.1% and incubated with Alexa Fluor 488 goat anti-mouse (Life Technologies), diluted 1:2,000 in BSA 2%, PBS/triton 0.2% for 30 min at 37° C. Cell nuclei were stained with DAPI. Glasses were then mounted with Dako Mounting medium and fluorescent signals were monitored under a Zeiss confocal microscope LSM510meta with a Zeiss EC Plan-Neofluar 40×/1.3 oil immersion objective.

To assess the percentage of transduced colonies, 10,000 cells from the sub-confluent transduced PGc pool were plated on a chamber slide and cultivated for 5 days as above. Chamber slides were fixed in methanol 100% for 10 min at −20° C. and immunofluorescence analysis was performed as above. Laminin 332-β positive colonies were counted under a Zeiss Microscope AXIO ImagerA1 with EC-Plan Neofluar 20×/0.5 objective.

In Situ Hybridization.

In situ hybridization (ISH) was performed on 10 μm skin sections. DIG-RNA probe synthesis was performed according to the manufacturer's instructions (Roche, DIG Labelling MIX). Primer pairs with Sp6/T7 promoter sequences (MWG Biotech) were used to obtain DNA templates for in vitro transcription. The following vector-specific primers were used: 5′-Sp6-AGTAACGCCATTTTGCAAGG-3′ (SEQ ID NO:56) (Tm 60° C.) and 5′-T7-AACAGAAGCGAGAAGCGAAC-3′ (SEQ ID NO:57) (Tm 58° C.)^{11,12}. OCT sections were fixed in PFA 4% and permeabilized with proteinase K 5 μg/ml and post-fixed in PFA 4%. Sections were then incubated in hybridization solution (50% formamide, 4×SSC, Yeast RNA 500 μg/ml, lx Denhard's solution, 2 mM EDTA, 10% dextran sulfate in DEPC treated water) at 37° C. for 1 h. DIG-probes were diluted in pre-heated hybridization solution at 80° C. for 2 min and added to the slice for 20 h at 37° C. Sections were washed, blocked in Antibody buffer (1% blocking reagent from Roche in PBS tween 0.1%) containing 10% sheep serum for 1 h at RT. Anti-DIG antibody 1:200 was diluted in the same blocking solution and added to the slide for 4 h at room temperature. Signals were developed with BM-Purple solution ON at RT until signal reached the desired intensity. Slices were then mounted in 70% glycerol and visualized with Zeiss Cell Observer microscope with EC-Plan Neofluar 20×/0.5 objective.

Statistical Analyses and Data Visualization.

Statistical analyses were implemented in R (v3.3.1, http://www.r-project.org/). FIG. 3d was generated using the ggplot2 R package (v2.2.1, https://cran.r-project.org/web/packages/ggplot2/index.html).

Results

The patient

In June 2015, a 7-year-old child (referred to as “H”) was admitted to the Burn Unit of the Children's Hospital, Ruhr-University, Bochum, Germany. He carried a homozygous acceptor splice site mutation (C1977-1G>A, IVS 14-1G>A) within intron 14 of LAMB3. Since birth, H developed blisters all over his body, particularly on limbs, back and flanks. His condition severely deteriorated six weeks before admission, due to infection with Staphylococcus aureus and Pseudomonas aeruginosa. Shortly after admission, H suffered complete epidermal loss on ˜60% of the total body surface area (TBSA). During the following weeks, all therapeutic approaches failed and H's short-term prognosis was unfavourable (Methods). After the parents' informed consent, the regional regulatory authorities and the ethical review board of the Ruhr-University authorised the compassionate use of combined ex vivo cell and gene therapy. At the time of the first surgery, H had complete epidermal loss on ˜80% TBSA (FIG. 1a).

Regeneration of a Functional Epidermis by Transgenic Epidermal Cultures

On Sep. 21 2015, a 4-cm² biopsy, taken from a non-blistering area of H's left inguinal region, was used to establish primary keratinocyte cultures, which were then transduced with a retroviral vector (RV) expressing the full-length LAMB3 cDNA under the control of the Moloney leukaemia virus (MLV) long terminal repeat¹³ (Methods, FIG. 5). Sequentially, 0.85 m² transgenic epidermal grafts, enough to cover all H's denuded body surface, were applied on a properly prepared dermal wound bed (FIG. 6a). All limbs, the entire back (including flanks) and some of the remaining denuded areas were grafted on Oct. 19 2015, Nov. 23 2015, and Jan. 26 2016, respectively.

Previously, transgenic epidermal sheets were cultivated on plastic, enzymatically detached from the vessel and mounted on a non-adhering gauze¹⁰⁻¹². Keratinocyte cultivation on a fibrin substrate—currently used to treat massive skin and ocular burns^{6,8,9}—eliminates cumbersome procedures for graft preparation and transplantation and avoids epidermal shrinking, allowing the production of larger grafts using the same number of clonogenic cells needed to produce plastic-cultured grafts. Since degradation of fibrin after transplantation, which is critical to allow cell engraftment, was never assessed in a JEB wound bed, at the first surgery we compared plastic- and fibrin-cultured grafts (Methods).

The left arm received plastic-cultured grafts (FIG. 6b, asterisks). Upon removal of the non-adhering gauze (10 days post-grafting, FIG. 6c, arrows), epidermal engraftment was evident (asterisks). Epidermal regeneration, evaluated at 1 month, was stable and complete (FIG. 6d). The left leg received both plastic- and fibrin-cultured grafts (FIG. 6e, asterisk and arrow, respectively), both of which showed full engraftment at 10 days (FIG. 6f, asterisk and arrow, respectively) and complete epidermal regeneration at 1 month (FIG. 6f, inset). Similar data were obtained on the other limbs. Thus, on Nov. 23 2015, H's denuded back (FIG. 6g) received only fibrin-cultured grafts (inset). As shown in FIG. 6h, virtually complete epidermal regeneration was observed at 1 month, with the exception of some areas (asterisks), some of which contained islands of newly formed epidermis (arrows). Over the following weeks, the regenerated epidermis surrounding the open lesions and those epidermal islands spread and covered most of the denuded areas (FIG. 6i). On Jan. 26 2016, we transplanted the remaining defects on flanks, thorax, right thigh, right hand and shoulders. Epidermal regeneration was attained in most of those areas.

Thus, ˜80% of H's TBSA was restored by the transgenic epidermis. During the 21 months follow-up (over 20 epidermal renewing cycles), the regenerated epidermis firmly adhered to the underlying dermis, even after induced mechanical stress (FIG. 1b), healed normally and did not form blisters, also in areas where follow-up biopsies were taken (FIG. 1c, arrow). H was discharged in February 2016 and is currently leading a normal social life. His epidermis is currently stable, robust, does not blister, itch, or require ointment or medications.

Ten punch biopsies were randomly taken, 4, 8 and 21 months after grafting. The epidermis had normal morphology and we could not detect blisters, erosions or epidermal detachment from the underlying dermis (Data FIG. 7a). In situ hybridization using a vector specific t-LAMB3 probe showed that the regenerated epidermis consisted only of transgenic keratinocytes (FIG. 2a). At admission, laminin 332-β3 was barely detectable in H's skin (FIG. 2b). In contrast, control and transgenic epidermis expressed virtually identical amounts of laminin 332-β3, which was properly located at the epidermal-dermal junction (FIG. 2b). The basal lamina contained normal amounts of laminin 332 α3 and γ2 chains and α6β4, all of which were strongly decreased at admission (FIG. 7b). Thus, transduced keratinocytes could restore a proper adhesion machinery (FIG. 7c). Indeed, the transgenic epidermis revealed normal thickness and continuity of the basement membrane (FIG. 2c, arrowheads) and normal morphology of hemidesmosomes (FIG. 2c, arrows). At 21 months follow-up, H's serum did not contain autoantibodies directed against the basement-membrane zone (FIG. 9).

In summary, transgenic epidermal cultures generated an entire functional epidermis in a JEB patient. This is consistent with the notion that keratinocyte cultures have been used for decades to successfully treat life-threatened burn victims on up to 98% of TBSA^{5,6,9,14}. It can be argued that H's clinical picture (massive epidermal loss, critical conditions, poor short-term prognosis) was unusual and our aggressive surgery (mandatory for H) unthinkable for the clinical course of most EB patients. But progressive replacement of diseased epidermis can be attained in multiple, less invasive surgical interventions on more limited body areas. EB has the advantage of a preserved dermis (not available in deep burns), which allows good functional and cosmetic outcomes. This approach would be optimal for newly diagnosed patients early in their childhood. A bank of transduced epidermal stem cells taken at birth could be used to treat skin lesions while they develop, thus preventing, rather than restoring, the devastating clinical manifestations rising through adulthood. Currently, combined ex vivo cell and gene therapy cannot be applied to lesions of the internal mucosae, which, however, are usually more manageable than those on skin, perhaps with the exception of oesophageal strictures.

Integration Profile of Transgenic Epidermis

Pre-graft transgenic cultures (PGc) were generated by ˜8.7×10⁶ primary clonogenic cells and consisted of 2.2×10⁸ keratinocytes (divided in 36 vials), ˜45% of which were seeded to prepare 0.85 m² transgenic epidermal grafts (FIG. 5).

To investigate the genome-wide integration profile, 3 PGc samples were sequenced using two independent LTR-primers (i.e., 3pIN and 3pOUT, for library enrichment (n=12; see Methods).

High-throughput sequencing recovered a total of 174.9M read pairs and the libraries obtained using the two LTR-primers showed similar number of reads and comparable insertion counts (Pearson R>0.92, p<0.005). After merging all integration sites from the two independent priming systems, we identified 27,303 integrations in PGc (FIG. 3a, bars) with an average coverage of 2.5 reads/insertion (FIG. 3a, lines). The same analysis was performed on primary cultures initiated from 3 biopsies (˜0.5 cm² each) taken at 4 (left leg) and 8 (right arm and left leg) months after grafting, referred to as 4Mc, 8Mc₁, and 8Mc₂, respectively (Methods).

Strikingly, we detected only 400, 206, and 413 integrations in 4Mc, 8Mc₁, and 8Mc₂, respectively (FIG. 3a, bars) with an average coverage of 27.3, 19.5, and 20.4 (FIG. 3a, lines).

To exclude that the major difference in the number of integrations found in pre- and post-graft samples could be ascribable to PCR reactions causing unbalanced representation of event-specific amplicons, or to spatiality-effect of punch biopsies, we estimated the expected number of PGc, 4Mc, 8Mc₁, and 8Mc₂ integrations using the Chapman-Wilson capture-recapture model on the data obtained from the independent libraries (Methods)¹⁵. In PGc, the model estimated 65,030±2,120 integrations, i.e. approximately twice the actual number of detected insertions. The same model estimated 457±31, 323±50, and 457±24, independent integrations in 4Mc, 8Mc₁, and 8Mc₂, respectively (confidence level of 99%, α=0.01), which is highly consistent with the number of events actually detected. Of note, 58%, 43% and 37% of 4Mc, 8Mc₁ and 8Mc₂ integrations, respectively, were identified in PGc (FIG. 3b), which is consistent with the percentage (˜50%) of insertions detected in PGc by NGS analysis.

Integrations were mapped to promoters (defined as 5 kb regions upstream the transcription start site of RefSeq genes), exons, introns, and intergenic regions. In all pre- and post-graft samples, ˜10% of events were located within promoters. The majority of integrations were either intronic (˜47%) or intergenic (˜38%) and less than 5% were found in exons (FIG. 3c, left panel). We also annotated integrations in epigenetically defined transcriptional regulatory elements (Methods). As shown in FIG. 3c (right panel), ˜27% of integrations were associated to active promoters or enhancers and no significant difference in the distribution of insertions was detected in pre- and post-graft samples (p-value>0.05; Pearson's Chi-squared test). Thus, the integration pattern was maintained in vivo and epidermal renewal did not determine any clonal selection.

Genes containing an integration were not functionally enriched in Gene Ontology categories related to cancer-associated biological processes¹⁶, with the exception of cell migration and small GTPase mediated signal transduction (FIG. 3d and Table 1). These findings are however expected, since our culture conditions are optimized to foster keratinocyte proliferation and migration, to sustain clonogenic cells and to avoid premature clonal conversion and terminal differentiation, all of which are instrumental for the proper clinical performance of cultured epidermal grafts¹⁴. Thus, similarly to what has been reported in transgenic hematopoietic stem cells^{17,18}, our high-throughput analyses revealed a cell-specific vector preference that is related to the host cell status in terms of chromatin state and transcriptional activity at the time of transduction¹⁹.

MLV-RV vectors raised concerns about insertional genotoxicity, which has been reported with hematopoietic stem cells, but in specific disease contexts^{17,20-22}. Indeed, a γRV vector, similar to ours, obtained a marketing authorization for ex vivo gene therapy of adenosine deaminase severe combined immunodeficiency and has been approved for PhaseI/II clinical trials on RDEB (https://clinicaltrials.gov/ct2/show/NCT02984085)²³. H's integration profile confirmed absence of clonal selection both in vitro and in vivo. Likewise, we never observed immortalization events related to specific proviral integrations in many serially cultivated MLV-RV-transduced keratinocytes (unpublished data). Two JEB patients, receiving a total of ˜1×10⁷ clonogenic transgenic keratinocytes in selected body sites (3.5 and 12 years follow-up)¹⁰⁻¹², and H, receiving ˜3.9×10⁸ transgenic clonogenic cells all over his body (FIG. 5), did not manifest tumour development or other related adverse events. Therefore, based on in vivo data, the frequency of a detectable transformation event (if any) in MLV-RV-transduced keratinocytes would be less than 1 out of 1×10⁷ during the first 12 years follow-up. Although H's follow-up is shorter and does not allow drawing definitive conclusions, the frequency of detectable insertional mutagenesis events to date is less than 1 out of 3.9×10⁸. In evaluating the risk/benefit ratio, it should also be considered that severely affected JEB patients are likely to develop aggressive squamous cell carcinoma as a consequence of the progression of the disease.

The Transgenic Epidermis is Sustained by Self-Renewing Stem Cells (Holoclones).

The percentage of clonogenic cells, including holoclones, remained relatively constant during the massive cell expansion needed to produce the grafts (FIG. 5). H received ˜3.9×10⁸ clonogenic cells, ˜1.6×10⁷ of which were holoclone-forming cells, to cover ˜0.85 m² of his body (FIG. 4a, FIG. 5). Thus, ˜4.4×10⁴/cm² clonogenic cells or ˜1.9×10³/cm² stem cells were transplanted on H's body surface (FIG. 4a).

If originally transduced clonogenic cells were all long-lived equipotent progenitors, (i) we would have recovered thousands of integrations per cm² of regenerated epidermis; (ii) all clonogenic cells contained in 4Mc, 8Mc₁ and 8Mc₂ cultures would have independent integrations, irrespectively of the clonal type. Instead, if the transgenic epidermis was sustained only by a restricted number of long-lived stem cells (continuously generating pools of TA progenitors), (i) we would have recovered, at most, only few hundreds of integrations per cm²; (ii) mero- and paraclones contained in 4Mc, 8Mc₁ and 8Mc₂ cultures would have the same integrations found in the corresponding holoclones.

The number of integrations detected in post-graft cultures (FIG. 3a) is consistent with the number of stem cells that have been transplanted (FIG. 4a), hence it strongly supports the latter hypothesis, which was verified by proviral analyses at clonal level (FIG. 9) on PGc, 4Mc and 8Mc₁. A total of 686 clones (41 holoclones and 645 mero/paraclones) were analysed. PGc, 4Mc and 8Mc₁ 5 generated 20, 14 and 7 holoclones and 259, 263 and 123 mero/paraclones, respectively. Thus, PGc, 4Mc and 8Mc₁ contained 7.2%, 5.0% and 5.4% holoclone-forming cells, respectively. Each clone was cultivated for further analysis. Libraries of vector-genome junctions, generated by linear-amplification-mediated (LAM) PCR followed by pyrosequencing, retrieved 31 independent integrations unambiguously mapped on the genome of holoclones (Table 2).

TABLE 2
Genomic and functional annotations of integrations in holoclones

| | | | | | | | Annotation to | |
|---|---|---|---|---|---|---|---|---|
| | | | | | Annotation to | | regulatory | Recovered |
| Sample | chr | start | end | ID Holoclone | genes | Gene symbol | elements | in PGc |
| PGc | chr5 | 131410002 | 131410003 | PGc_H1 | Intron | CSF2 | no mark | no |
| | chr2 | 144859325 | 144859326 | PGc_H2 | Intron | GTDC1 | no mark | no |
| | chr4 | 101941589 | 101941590 | PGc_H3 | Intergenic | — | weak enhancer | no |
| | chr4 | 39355299 | 39355300 | PGc_H4 | Intron | RFG1 | weak enhancer | no |
| | chr19 | 17908000 | 17908001 | PGc_H5 | Intron | B3GNT3 | no mark | no |
| | chr19 | 42615156 | 42615157 | PGc_H6 | Intron | POU2F2 | no mark | no |
| | chr5 | 150977858 | 150977859 | PGc_H7* | Intergenic | — | active enhancer | yes |
| | chr7 | 80832738 | 80832739 | PGc_H7* | Intron | TBCD | active enhancer | yes |
| | chr16 | 56726522 | 56726523 | PGc_H7* | Intergenic | — | no mark | ye |
| | chr2 | 899619 | 8999620 | PGc_H8 | Intron | MBOAT2 | no mark | no |
| | chr3 | 47024025 | 47024026 | PGc_H9 | Promoter | CCDC12 | active promoter | yes |
| | chrY | 18367597 | 18367598 | PGc_H10 | Intergenic | — | no mark | no |
| | chr6 | 160458524 | 160458525 | PGc_H11 | Intron | IGF2R | no mark | no |
| | chr14 | 91711334 | 91711335 | PGc_H12 | Promoter | GPR68 | active promoter | yes |
| | chr11 | 13946563 | 13946564 | PGc_H13 | Promoter | LOC101928132 | no mark | yes |
| | chr14 | 33789922 | 33789923 | PGc_H14 | Intron | NPAS3 | no mark | no |
| | chr13 | 20693331 | 20693332 | PGc_H15 | Intergenic | — | weak enhancer | no |
| | chr6 | 136930722 | 136930723 | PGc_H16 | Intron | MAP3K5 | weak enhancer | yes |
| | chr18 | 65398639 | 65398640 | PGc_H17 | Intron | LOC643542 | no mark | no |
| | chr4 | 11625725 | 11625726 | PGc_H18 | Intergenic | — | active enhancer | no |
| | chr20 | 22743911 | 22743912 | PGc_H19 | Intergenic | — | no mark | yes |
| | chr8 | 48293010 | 48293011 | PGc_H20 | Intron | SPIDR | active enhancer | no |
| 4Mc | chr1 | 183130951 | 183130952 | 4Mc_H1-11** | Intergenic | — | no mark | yes |
| | chr9 | 103188807 | 103188808 | 4Mc_H1-11** | Promoter | MSANTD3 | active promoter | yes |
| | chr14 | 105213201 | 105213202 | 4Mc_H12 | Intron | ADSSL1 | no mark | no |
| | chr15 | 39577423 | 39577424 | 4Mc_H13 | Intergenic | — | no mark | yes |
| 8Mc₁ | chr8 | 67025314 | 67025315 | 8Mc1_H1-2 | Intergenic | — | active enhancer | yes |
| | chr9 | 125129763 | 125129764 | 8Mc1_H3 | Promoter | PTGS1 | no mark | yes |
| | chr17 | 76158277 | 76158278 | 8Mc1_H4-5 | Intron | C17orf99 | no mark | no |
| | chrX | 114601642 | 114601643 | 8Mc1_H6 | Intergenic | — | no mark | yes |
| | chr5 | 135342207 | 135342208 | 8Mc1_H7 | Intergenic | — | no mark | yes |

*holoclone with three different integrations
**holoclone with two different integrations

One holoclone (4Mc) was untransduced, 28, 11 and 1 holoclones contained 1,2 and 3 integrations, respectively. Eleven holoclones in 4Mc shared the same integration pattern. The same happened for two couples of holoclones in 8Mc₁. Holoclones' copy numbers were confirmed by RTq-PCR (FIG. 10). Strikingly, 75% and 80% of integrations found in 4Mc and 8Mc₁ holoclones were retrieved in PGc, respectively (FIG. 4b), supporting the NGS-based survey as well as a representative sampling. The integration pattern observed in holoclones confirms absence of selection of specific integrations during epidermal renewal in vivo (FIG. 4c) and mirrors the pattern found in their parental cultures (FIG. 3c), including absence of genes associated to cell cycle control, cell death, or oncogenesis (FIG. 3d and Table 1).

TABLE 1
Enrichment of cancer-related biological process in genes harboring an insertion. Statistical
significant enrichments at a 95% confidence level (q-value ≤ 0.05 in a Fisher's
exact test) are in bold. GO categories were selected to represent the cancer hallmarks
described in Hanahan D, Weinberg R A. Cell. 2011 Mar. 4; 144(5): 646-74.

| Cancer-related | | | q-value (FDR) | | | |
|---|---|---|---|---|---|---|
| biological process | GO ID | Description | PGc | 4Mc | 8Mc₁ | 8Mc₂ |
| Cell death | GO:0070265 | necrotic cell death | 0.28 | 0.58 | 0.56 | 0.65 |
| and apoptosis | GO:0010939 | regulation of necrotic cell death | 0.31 | 0.53 | 0.53 | 0.64 |
| | GO:0097300 | programmed necrotic cell death | 0.25 | 0.54 | 0.53 | 0.66 |
| | GO:2001233 | regulation of apoptotic signaling pathway | — | 0.52 | 0.67 | 0.72 |
| DNA repair | GO:0006282 | regulation of DNA repair | 0.06 | 0.67 | — | — |
| | GO:0006298 | mismatch repair | 0.53 | 0.54 | — | 0.66 |
| | GO:0006302 | double-strand break repair | 0.64 | 0.57 | 0.72 | 0.91 |
| | GO:0006289 | nucleotide-excision repair | 0.82 | 0.75 | — | 0.83 |
| | GO:0036297 | interstrend cross-link repair | 0.84 | 0.58 | — | 0.72 |
| Angiogenesis | GO:0001525 | angiogenesis | 9.54E−05 | 0.52 | 0.59 | 0.74 |
| | GO:0045765 | regulation of angiogenesis | 0.53 | 0.73 | 0.73 | 0.72 |
| Migration | GO:0090130 | tissue migration | 7.82E−08 | 0.50 | 0.53 | 0.04 |
| | GO:0090132 | epithelium migration | 3.64E−06 | 0.50 | 0.53 | 0.04 |
| | GO:0010631 | epithelial cell migration | 3.26E−06 | 0.49 | 0.53 | 0.04 |
| | GO:0010632 | regulation of epithelial cell migration | 2.43E−06 | 0.44 | 0.53 | 0.05 |
| | GO:0051546 | keratinocyte migration | 0.22 | — | 0.53 | 0.65 |
| | GO:0001667 | ambeboidal-type cell migration | 3.19E−08 | 0.52 | 0.53 | 0.06 |
| Inflammation | GO:0002526 | acute inflammatory response | 0.85 | 0.58 | 0.63 | — |
| | GO:0002544 | chronic inflammatory response | 0.82 | 0.45 | — | — |
| | GO:0050727 | regulation of inflammatory response | 0.80 | 0.69 | 0.61 | 0.80 |
| | GO:0000723 | telomere maintenance | 0.48 | 0.77 | 0.63 | 0.72 |
| Telomerase activity | GO:0007004 | telomere maintenance via telomerase | 0.38 | — | — | 0.73 |
| | GO:0032204 | regulation of telomere maintenance | 0.69 | — | — | 0.65 |
| | GO:0051972 | regulation of telomerase activity | 0 66 | — | — | — |
| Cell cycle | GO 0000075 | cell cycle checkpoint | 0.14 | 0.60 | 0.74 | — |
| | GO:1901976 | regulation of cell cycle checkpoint | 0.18 | — | — | — |
| | GO:1901987 | regulation of cell cycle phase transition | 0.02 | 0.48 | 0.83 | — |
| | GO:0045786 | negative regulation of cell cycle | 0.01 | 0.57 | 0.60 | — |
| Proliferation | GO:0050673 | epithelial cell proliferation | 4.06E−03 | 0.52 | 0.65 | 0.75 |
| | GO:0050678 | regulation epithelial cell proliferation | 0.01 | 0.52 | 0.60 | 0.72 |
| | GO:0043616 | keratinocyte proliferation | 1.24E−03 | 0.56 | 0.55 | — |
| | GO:0010837 | regulation of keratinocyte proliferation | 0.01 | 0.54 | 0.53 | — |
| | GO:0072089 | stem cell proliferation | 0.25 | 0.73 | 0.60 | — |
| Glycolysis | GO:0006096 | glycolytic process | 0.15 | 0.65 | — | 0.75 |
| | GO:0006110 | regulation of glycolytic process | 0.22 | 0.54 | — | 0.66 |

Clonal tracing was then performed by PCR, using genomic coordinates of holoclone insertions. As expected, the vast majority of PGc meroclones and paraclones (91%) did not contain the same integrations detected in the corresponding holoclones (FIG. 4d, PGc). Such percentage decreased to 37% already at 4 months after grafting (FIG. 4d, 4Mc). Strikingly, virtually the entire clonogenic population of primary keratinocyte cultures established at 8 months contained the same integrations detected in the corresponding holoclones (FIG. 4d, 8Mc₁). Thus, the in vivo half-live of TA progenitors is of approximately 3-4 months. These data formally show that the regenerated epidermis is sustained only by long-lived stem cells (holoclones) and underpins the notion that meroclones and paraclones are short-lived progenitors continuously generated by the holoclones, both in vitro and in vivo. The high percentage of holoclone integrations retrieved in PGc, together with the number of shared events across cultures (FIG. 3b), suggests that the average coverage of the NGS analysis in PGc allowed to preferentially identify integrations in holoclones and in TA cells deriving from such holoclones already during the cultivation process.

In summary, as depicted in FIG. 11, altogether these findings demonstrate that (i) PGc consisted of a mixture of independent transgenic holoclones, meroclones and paraclones, (ii) meroclones and paraclones (which can be isolated directly from a skin biopsy, our unpublished data) are TA progenitors, do not self-renew and are progressively lost during cultivation and in vivo epidermal renewal, hence do not contribute to long-term maintenance of the epidermis; (iii) the transgenic epidermis is sustained only by long-lived stem cells detected as holoclones; (iv) founder stem cells contained in the original primary culture must have gone extensive self-renewal (in vitro and in vivo) to ultimately sustain the regenerated epidermis, as confirmed by the number of shared events across samples and across holoclones.

### DISCUSSION

The entire epidermis of a JEB patient can be replaced by autologous transgenic epidermal cultures harbouring an appropriate number of stem cells. Both stem and TA progenitors are instrumental for proper tissue regeneration in mammals²⁴. However, the nature and the properties of mammalian epidermal stem cells and TA progenitors are a matter of debate^{25,26}. Although epidermal cultures have been used for 30 years in the clinic¹⁴, a formal proof of the engraftment of cultured stem cells has been difficult to obtain. Similarly, the identification of holoclones as human epithelial stem cells and mero/paraclones as TA progenitors and their role in long-term human epithelial regeneration have been inferred from compelling, yet indirect evidence^{6,8,9, 27}. Using integrations as clonal genetic marks, we show that the vast majority of TA progenitors are progressively lost within a few months after grafting and the regenerated epidermis is indeed sustained only by a limited number of long-lasting, self-renewing stem cells. Similar data have been produced with transgenic hematopoietic stem cells²⁸. This notion argues against a model positing the existence of a population of equipotent epidermal progenitors that directly generate differentiated cells during the lifetime of the animal²⁵ and fosters a model where specific stem cells persist during the lifetime of the human and contribute to both renewal and repair by giving rise to pools of progenitors that persist for various periods of time, replenish differentiated cells and make short-term contribution to wound healing²⁶. Hence, the essential feature of any cultured epithelial grafts is the presence (and preservation) of an adequate number of holoclone-forming cells. The notion that the transgenic epidermis is sustained only by engrafted stem cells further decreases the potential risk of insertional oncogenesis.

In conclusion, transgenic epidermal stem cells can regenerate a fully functional epidermis virtually indistinguishable from a normal epidermis, so far in the absence of related adverse events. The different forms of EB affect approximately 500,000 people worldwide (http://www.debra.org). The successful outcome of this study paves the road to gene therapy of other types of EB and provides a blueprint that can be applied to other stem cell-mediated combined ex vivo cell and gene therapies.

### REFERENCES

1 Fine, J. D. et al. Inherited epidermolysis bullosa: updated recommendations on diagnosis and classification. J Am Acad Dermatol 70, 1103-1126, doi:10.1016/j.jaad.2014.01.903 (2014).
2 Fine, J. D., Johnson, L. B., Weiner, M. & Suchindran, C. Cause-specific risks of childhood death in inherited epidermolysis bullosa. J Pediatr 152, 276-280, doi: 10.1016/j.jpeds.2007.06.039 (2008).
3 Barrandon, Y. & Green, H. Three clonal types of keratinocyte with different capacities for multiplication. Proceedings of the National Academy of Sciences of the United States of America 84, 2302-2306 (1987).
4 Pellegrini, G. et al. Location and clonal analysis of stem cells and their differentiated progeny in the human ocular surface. J Cell Biol 145, 769-782 (1999).
Gallico, G. G., 3rd, O'Connor, N. E., Compton, C. C., Kehinde, O. & Green, H. Permanent coverage of large burn wounds with autologous cultured human epithelium. The New England journal of medicine 311, 448-451, doi:10.1056/NEJM198408163110706 (1984).
6 Pellegrini, G. et al. The control of epidermal stem cells (holoclones) in the treatment of massive full-thickness burns with autologous keratinocytes cultured on fibrin. Transplantation 68, 868-879 (1999).
7 Pellegrini, G. et al. Long-term restoration of damaged corneal surfaces with autologous cultivated corneal epithelium. Lancet 349, 990-993, doi: 10.1016/S 0140-6736(96)11188-0 (1997).
8 Rama, P. et al. Limbal stem-cell therapy and long-term corneal regeneration. The New England journal of medicine 363, 147-155, doi:10.1056/NEJMoa0905955 (2010).
9 Ronfard, V., Rives, J. M., Neveux, Y., Carsin, H. & Barrandon, Y. Long-term regeneration of human epidermis on third degree burns transplanted with autologous cultured epithelium grown on a fibrin matrix. Transplantation 70, 1588-1598 (2000).
10 Bauer, J. W. et al. Closure of a Large Chronic Wound through Transplantation of Gene-Corrected Epidermal Stem Cells. Journal of Investigative Dermatology 137, 778-781, doi: 10.1016/j.jid.2016.10.038.
11 De Rosa, L. et al. Long-term stability and safety of transgenic cultured epidermal stem cells in gene therapy of junctional epidermolysis bullosa. Stem Cell Reports 2, 1-8, doi:10.1016/j.stemcr.2013.11.001 (2014).
12 Mavilio, F. et al. Correction of junctional epidermolysis bullosa by transplantation of genetically modified epidermal stem cells. Nat Med 12, 1397-1402, doi: 10.1038/nm1504 (2006).
13 Markowitz, D., Goff, S. & Bank, A. Construction and use of a safe and efficient amphotropic packaging cell line. Virology 167, 400-406 (1988).
14 De Luca, M., Pellegrini, G. & Green, H. Regeneration of squamous epithelia from stem cells of cultured grafts. Regenerative medicine 1, 45-57, doi: 10.2217/17460751.1.1.45 (2006).
15 Chapman, D. G. & Robbins, H. Minimum Variance Estimation Without Regularity Assumptions. The Annals of Mathematical Statistics 22, 581-586 (1951).
16 Hanahan, D. & Weinberg, R. A. Hallmarks of cancer: the next generation. Cell 144, 646-674, doi:10.1016/j.cell.2011.02.013 (2011).
17 Aiuti, A. et al. Gene therapy for immunodeficiency due to adenosine deaminase deficiency. The New England journal of medicine 360, 447-458, doi:10.1056/NEJMoa0805817 (2009).
18 Biasco, L. et al. Integration profile of retroviral vector in gene therapy treated patients is cell-specific according to gene expression and chromatin conformation of target cell. EMBO Mol Med 3, 89-101, doi:10.1002/emmm.201000108 (2011).
19 Cavazza, A. et al. Self-inactivating MLV vectors have a reduced genotoxic profile in human epidermal keratinocytes. Gene Ther 20, 949-957, doi:10.1038/gt.2013.18 (2013).
20 Hacein-Bey-Abina, S. et al. Insertional oncogenesis in 4 patients after retrovirus-mediated gene therapy of SCID-X1. J Clin Invest 118, 3132-3142, doi: 10.1172/JCI35700 (2008).
21 Hacein-Bey-Abina, S. et al. A serious adverse event after successful gene therapy for X-linked severe combined immunodeficiency. The New England journal of medicine 348, 255-256, doi: 10.1056/NEJM200301163480314 (2003).
22 Howe, S. J. et al. Insertional mutagenesis combined with acquired somatic mutations causes leukemogenesis following gene therapy of SCID-X1 patients. J Clin Invest 118, 3143-3150, doi: 10.1172/JCI35798 (2008).
23 Siprashvili, Z. et al. Safety and Wound Outcomes Following Genetically Corrected Autologous Epidermal Grafts in Patients With Recessive Dystrophic Epidermolysis Bullosa. JAMA 316, 1808-1817, doi:10.1001/jama.2016.15588 (2016).
24 Hsu, Y. C., Li, L. & Fuchs, E. Transit-amplifying cells orchestrate stem cell activity and tissue regeneration. Cell 157, 935-949, doi:10.1016/j.cell.2014.02.057 (2014).
25 Clayton, E. et al. A single type of progenitor cell maintains normal epidermis. Nature 446, 185-189, doi:10.1038/nature05574 (2007).
26 Mascre, G. et al. Distinct contribution of stem and progenitor cells to epidermal maintenance. Nature 489, 257-262, doi: 10.1038/nature 1393 (2012).
27 Pellegrini, G. et al. Biological parameters determining the clinical outcome of autologous cultures of limbal stem cells. Regenerative medicine 8, 553-567, doi:10.2217/rme.13.43 (2013).
28 Biasco, L. et al. In Vivo Tracking of Human Hematopoiesis Reveals Patterns of Clonal Dynamics during Early and Steady-State Reconstitution Phases. Cell Stem Cell 19, 107-119, doi:10.1016/j.stem.2016.04.016 (2016).
29 Schwieger-Briel, A. et al. Instrument for scoring clinical outcome of research for epidermolysis bullosa: a consensus-generated clinical research tool. Pediatr Dermatol 32, 41-52, doi:10.1111/pde.12317 (2015).
30 Hemdon, D. N. et al. Long-term propranolol use in severely burned pediatric patients: a randomized controlled study. Ann Surg 256, 402-411, doi: 10.1097/SLA.0b013e318265427e (2012).
31 Goldschneider, K. R. et al. Pain care for patients with epidermolysis bullosa: best care practice guidelines. BMC Med 12, 178, doi:10.1186/s12916-014-0178-2 (2014).
32 Rodriguez, N. A., Jeschke, M. G., Williams, F. N., Kamolz, L. P. & Hemdon, D. N. Nutrition in burns: Galveston contributions. JPEN J Parenter Enteral Nutr 35, 704-714, doi: 10.1177/0148607111417446 (2011).
33 Dellambra, E. et al. Corrective transduction of human epidermal stem cells in laminin-5-dependent junctional epidermolysis bullosa. Hum Gene Ther 9, 1359-1370, doi: 10.1089/hum.1998.9.9-1359 (1998).
34 Krall, W. J. et al. Increased levels of spliced RNA account for augmented expression from the MFG retroviral vector in hematopoietic cells. Gene Ther 3, 37-48 (1996).
Mathor, M. B. et al. Clonal analysis of stably transduced human epidermal stem cells in culture. Proceedings of the National Academy of Sciences of the United States of America 93, 10371-10376 (1996).
36 Mavilio, F. et al. Correction of junctional epidermolysis bullosa by transplantation of genetically modified epidermal stem cells. Nat Med 12, 1397-1402, doi:10.1038/nm1504 (2006).
37 Bauer, J. W. et al. Closure of a Large Chronic Wound through Transplantation of Gene-Corrected Epidermal Stem Cells. Journal of Investigative Dermatology 137, 778-781, doi:10.1016/j.jid.2016.10.038.
38 Guerra, L. et al. Treatment of “stable” vitiligo by Timedsurgery and transplantation of cultured epidermal autografts. Arch Dermatol 136, 1380-1389 (2000).
39 Barrandon, Y. & Green, H. Three clonal types of keratinocyte with different capacities for multiplication. Proceedings of the National Academy of Sciences of the United States of America 84, 2302-2306 (1987).
40 Martin, M. Cutadapt removes adapter sequences from high-throughput sequencing reads. 2011 17, doi:10.14806/ej.17.1.200 pp. 10-12 (2011).
41 Li, H. Aligning sequence reads, clone sequences and assembly contigs with BWA-MEM. Eprint Arxiv (2013).
42 Quinlan, A. R. BEDTools: The Swiss-Army Tool for Genome Feature Analysis. Curr Protoc Bioinformatics 47, 11 12 11-34, doi:10.1002/0471250953.bi1 112s47 (2014).
43 De Rosa, L. et al. Long-term stability and safety of transgenic cultured epidermal stem cells in gene therapy of junctional epidermolysis bullosa. Stem Cell Reports 2, 1-8, doi:10.1016/j.stemcr.2013.11.001 (2014).

1. A method for the treatment of Epidermolysis Bullosa (EB) comprising administering to a subject in need thereof a flap of genetically modified cells on a fibrin substrate, wherein said genetically modified cells are genetically modified with at least one heterologous nucleic acid comprising a nucleotide sequence encoding:
a) at least one chain selected from the group consisting of: β3, α3 and γ2 chain of laminin-332, and/or
b) collagen XVII and/or
c) at least one α6β4 integrin and/or
d) collagen VII and/or
e) keratin 5 and/or Keratin 14 and/or
f) Plectin.

2. A The method of claim 1, wherein the treatment promotes in vivo cell adhesion and/or in vivo cell growth and/or cell regeneration optionally in the repair or replacement of living tissue, in an EB patient.

3. The method according to claim 1, wherein the EB is Junctional Epidermolysis Bullosa (JEB).

4. The method according to claim 3, wherein the heterologous nucleic acid comprises a nucleotide sequence encoding laminin-332 β3 chain and/or collagen XVII.

5. The method according to claim 1, wherein:
a) the laminin-332 β3 chain comprises an amino acid sequence having at least 75% amino acid sequence identity to the amino acid sequence SEQ ID NO: 6 and/or
b) the collagen XVII comprises an amino acid sequence having at least 75% amino acid sequence identity to the amino acid sequence SEQ ID NO:4 and/or
c) the collagen VII comprises an amino acid sequence having at least 75% amino acid sequence identity to the amino acid sequence SEQ ID NO: 2.

6. The method according to claim 1, wherein said heterologous nucleic acid further comprises a promoter that is operably linked to the promoter, and/or wherein the promoter is heterologous to the encoding nucleotide sequence and/or said heterologous nucleic acid is under the control of virus long terminal repeat (LTR), optionally of retrovirus LTR, or of Moloney Leukaemia virus (MLV) LTR.

7. The method according to claim 1, wherein the genetically modified cells have been transduced with the at least one heterologous nucleic acid.

8. The method according to claim 7, wherein the transduction was carried out with a viral vector, optionally with a retroviral vector, said retroviral vector optionally selected from the group consisting of alpharetroviral vector, a gammaretroviral vector, a lentiviral vector and a spumaretroviral vector.

9. The method according to claim 1, wherein the flap is obtainable by an in vitro method, characterized by:
a) plating feeder cells on the upper surface of a fibrin substrate so as to obtain a fibrin substrate on which said feeder cells are adhered;
b) plating and cultivating to subconfluence said genetically modified cells on said fibrin substrate onto which feeder cells are adhered, said fibrin substrate being positioned on a solid support so that the cells do not interact with the surface of said support so as to obtain a flap of genetically modified cells adhered to said fibrin substrate; and
c) detaching the flap of genetically modified cells adhered to said fibrin substrate from the support in a form similar to a sheet to obtain a flap of genetically modified cells on fibrin substrate.

10. The method according to claim 9, wherein the feeder cells are plated on the fibrin substrate from 2 to 24 hours before plating the genetically modified cells.

11. The method according to claim 9, wherein the method further comprises:
before step c), the steps:
b′) removing the culture medium and/or
b″) washing the flap of genetically modified cells adhered to said fibrin substrate with a washing solution
and/or after step c), the step of:
d) placing the obtained flap of genetically modified cells on fibrin substrate in a transport container
and/or wherein the fibrin substrate has dimensions of from 0.32 cm² to 300 cm².

12. The flap of genetically modified cells on fibrin substrate for use according to claim 9, wherein the fibrin substrate comprises from about 20 to about 100 mg/ml of fibrinogen and from about 1 to about 10 IU/ml of thrombin.

13. The method according to claim 12, wherein the fibrin substrate comprises from about 20 to about 50 mg/ml of fibrinogen, optionally from about 20 to about 40 mg/ml of fibrinogen, and from about 3 to about 8 IU/ml of thrombin.

14. The method according to claim 13, wherein the fibrin substrate comprises from about 20 to about 25 mg/ml of fibrinogen and from about 2 to about 4 IU/ml of thrombin

15. The method according to claim 14, wherein the fibrin substrate comprises about 23.1 mg/ml of fibrinogen and about 3.1 IU/ml of thrombin.

16. The method flap according to claim 9 wherein said cells are epithelial cells, optionally primary epithelial cells deriving from stratified epithelia.

17. The method according to claim 16, wherein said cells are epidermal cells.

18. The method according to claim 16 wherein said cells are keratinocytes, optionally human primary keratinocytes isolated from biopsies.

19. The method according to claim 18, wherein the biopsy is a cutaneous biopsy isolated from a EB patient, optionally a JEB, simple EB (EBS), dystrophic EB (DAB) and Kindler syndrome patient, said EB patient optionally being the same patient subject to the treatment.


SEQUENCE LISTING (57 sequences)

SEQ ID NO: 1 - DNA, 8835 bp, Homo sapiens
atgacgctgcggcttctggtggccgcgctctgcgccgggatcctggcagaggcgccccgagtgcgagcccagcacagggagagagtgacctgcacgcgcctttacgccgctgacattgtgttcttactggatggctcctcatccattggccgcagcaatttccgcgaggtccgcagctttctcgaagggctggtgctgcctttctctggagcagccagtgcacagggtgtgcgctttgccacagtgcagtacagcgatgacccacggacagagttcggcctggatgcacttggctctgggggtgatgtgatccgcgccatccgtgagcttagctacaaggggggcaacactcgcacaggggctgcaattctccatgtggctgaccatgtcttcctgccccagctggcccgacctggtgtccccaaggtctgcatcctgatcacagacgggaagtcccaggacctggtggacacagctgcccaaaggctgaaggggcagggggtcaagctatttgctgtggggatcaagaatgctgaccctgaggagctgaagcgagttgcctcacagcccaccagtgacttcttcttcttcgtcaatgacttcagcatcttgaggacactactgcccctcgtttcccggagagtgtgcacgactgctggtggcgtgcctgtgacccgacctccggatgactcgacctctgctccacgagacctggtgctgtctgagccaagcagccaatccttgagagtacagtggacagcggccagtggccctgtgactggctacaaggtccagtacactcctctgacggggctgggacagccactgccgagtgagcggcaggaggtgaacgtcccagctggtgagaccagtgtgcggctgcggggtctccggccactgaccgagtaccaagtgactgtgattgccctctacgccaacagcatcggggaggctgtgagcgggacagctcggaccactgccctagaagggccggaactgaccatccagaataccacagcccacagcctcctggtggcctggcggagtgtgccaggtgccactggctaccgtgtgacatggcgggtcctcagtggtgggcccacacagcagcaggagctgggccctgggcagggttcagtgttgctgcgtgacttggagcctggcacggactatgaggtgaccgtgagcaccctatttggccgcagtgtggggcccgccacttccctgatggctcgcactgacgcttctgttgagcagaccctgcgcccggtcatcctgggccccacatccatcctcctttcctggaacttggtgcctgaggcccgtggctaccggttggaatggcggcgtgagactggcttggagccaccgcagaaggtggtactgccctctgatgtgacccgctaccagttggatgggctgcagccgggcactgagtaccgcctcacactctacactctgctggagggccacgaggtggccacccctgcaaccgtggttcccactggaccagagctgcctgtgagccctgtaacagacctgcaagccaccgagctgcccgggcagcgggtgcgagtgtcctggagcccagtccctggtgccacccagtaccgcatcattgtgcgcagcacccagggggttgagcggaccctggtgcttcctgggagtcagacagcattcgacttggatgacgttcaggctgggcttagctacactgtgcgggtgtctgctcgagtgggtccccgtgagggcagtgccagtgtcctcactgtccgccgggagccggaaactccacttgctgttccagggctgcgggttgtggtgtcagatgcaacgcgagtgagggtggcctggggacccgtccctggagccagtggatttcggattagctggagcacaggcagtggtccggagtccagccagacactgcccccagactctactgccacagacatcacagggctgcagcctggaaccacctaccaggtggctgtgtcggtactgcgaggcagagaggagggccctgctgcagtcatcgtggctcgaacggacccactgggcccagtgaggacggtccatgtgactcaggccagcagctcatctgtcaccattacctggaccagggttcctggcgccacaggatacagggtttcctggcactcagcccacggcccagagaaatcccagttggtttctggggaggccacggtggctgagctggatggactggagccagatactgagtatacggtgcatgtgagggcccatgtggctggcgtggatgggccccctgcctctgtggttgtgaggactgcccctgagcctgtgggtcgtgtgtcgaggctgcagatcctcaatgcttccagcgacgttctacggatcacctgggtaggggtcactggagccacagcttacagactggcctggggccggagtgaaggcggccccatgaggcaccagatactcccaggaaacacagactctgcagagatccggggtctcgaaggtggagtcagctactcagtgcgagtgactgcacttgtcggggaccgcgagggcacacctgtctccattgttgtcactacgccgcctgaggctccgccagccctggggacgcttcacgtggtgcagcgcggggagcactcgctgaggctgcgctgggagccggtgcccagagcgcagggcttccttctgcactggcaacctgagggtggccaggaacagtcccgggtcctggggcccgagctcagcagctatcacctggacgggctggagccagcgacacagtaccgcgtgaggctgagtgtcctagggccagctggagaagggccctctgcagaggtgactgcgcgcactgagtcacctcgtgttccaagcattgaactacgtgtggtggacacctcgatcgactcggtgactttggcctggactccagtgtccagggcatccagctacatcctatcctggcggccactcagaggccctggccaggaagtgcctgggtccccgcagacacttccagggatctcaagctcccagcgggtgacagggctagagcctggcgtctcttacatcttctccctgacgcctgtcctggatggtgtgcggggtcctgaggcatctgtcacacagacgccagtgtgcccccgtggcctggcggatgtggtgttcctaccacatgccactcaagacaatgctcaccgtgcggaggctacgaggagggtcctggagcgtctggtgttggcacttgggcctcttgggccacaggcagttcaggttggcctgctgtcttacagtcatcggccctccccactgttcccactgaatggctcccatgaccttggcattatcttgcaaaggatccgtgacatgccctacatggacccaagtgggaacaacctgggcacagccgtggtcacagctcacagatacatgttggcaccagatgctcctgggcgccgccagcacgtaccaggggtgatggttctgctagtggatgaacccttgagaggtgacatattcagccccatccgtgaggcccaggcttctgggcttaatgtggtgatgttgggaatggctggagcggacccagagcagctgcgtcgcttggcgccgggtatggactctgtccagaccttcttcgccgtggatgatgggccaagcctggaccaggcagtcagtggtctggccacagccctgtgtcaggcatccttcactactcagccccggccagagccctgcccagtgtattgtccaaagggccagaagggggaacctggagagatgggcctgagaggacaagttgggcctcctggcgaccctggcctcccgggcaggaccggtgctcccggcccccaggggccccctggaagtgccactgccaagggcgagaggggcttccctggagcagatgggcgtccaggcagccctggccgcgccgggaatcctgggacccctggagcccctggcctaaagggctctccagggttgcctggccctcgtggggacccgggagagcgaggacctcgaggcccaaagggggagccgggggctcccggacaagtcatcggaggtgaaggacctgggcttcctgggcggaaaggggaccctggaccatcgggcccccctggacctcgtggaccactgggggacccaggaccccgtggccccccagggcttcctggaacagccatgaagggtgacaaaggcgatcgtggggagcggggtccccctggaccaggtgaaggtggcattgctcctggggagcctgggctgccgggtcttcccggaagccctggaccccaaggccccgttggcccccctggaaagaaaggagaaaaaggtgactctgaggatggagctccaggcctcccaggacaacctgggtctccgggtgagcagggcccacggggacctcctggagctattggccccaaaggtgaccggggctttccagggcccctgggtgaggctggagagaagggcgaacgtggacccccaggcccagcgggatcccgggggctgccaggggttgctggacgtcctggagccaagggtcctgaagggccaccaggacccactggccgccaaggagagaagggggagcctggtcgccctggggaccctgcagtggtgggacctgctgttgctggacccaaaggagaaaagggagatgtggggcccgctgggcccagaggagctaccggagtccaaggggaacggggcccacccggcttggttcttcctggagaccctggccccaagggagaccctggagaccggggtcccattggccttactggcagagcaggacccccaggtgactcagggcctcctggagagaagggagaccctgggcggcctggccccccaggacctgttggcccccgaggacgagatggtgaagttggagagaaaggtgacgagggtcctccgggtgacccgggtttgcctggaaaagcaggcgagcgtggccttcggggggcacctggagttcgggggcctgtgggtgaaaagggagaccagggagatcctggagaggatggacgaaatggcagccctggatcatctggacccaagggtgaccgtggggagccgggtcccccaggacccccgggacggctggtagacacaggacctggagccagagagaagggagagcctggggaccgcggacaagagggtcctcgagggcccaagggtgatcctggcctccctggagcccctggggaaaggggcattgaagggtttcggggacccccaggcccacagggggacccaggtgtccgaggcccagcaggagaaaagggtgaccggggtccccctgggctggatggccggagcggactggatgggaaaccaggagccgctgggccctctgggccgaatggtgctgcaggcaaagctggggacccagggagagacgggcttccaggcctccgtggagaacagggcctccctggcccctctggtccccctggattaccgggaaagccaggcgaggatggcaaacctggcctgaatggaaaaaacggagaacctggggaccctggagaagacgggaggaagggagagaaaggagattcaggcgcctctgggagagaaggtcgtgatggccccaagggtgagcgtggagctcctggtatccttggaccccaggggcctccaggcctcccagggccagtgggccctcctggccagggttttcctggtgtcccaggaggcacgggccccaagggtgaccgtggggagactggatccaaaggggagcagggcctccctggagagcgtggcctgcgaggagagcctggaagtgtgccgaatgtggatcggttgctggaaactgctggcatcaaggcatctgccctgcgggagatcgtggagacctgggatgagagctctggtagcttcctgcctgtgcccgaacggcgtcgaggccccaagggggactcaggcgaacagggccccccaggcaaggagggccccatcggctttcctggagaacgcgggctgaagggcgaccgtggagaccctggccctcaggggccacctggtctggcccttggggagaggggcccccccgggccttccggccttgccggggagcctggaaagcctggtattcccgggctcccaggcagggctgggggtgtgggagaggcaggaaggccaggagagaggggagaacggggagagaaaggagaacgtggagaacagggcagagatggccctcctggactccctggaacccctgggccccccggaccccctggccccaaggtgtctgtggatgagccaggtcctggactctctggagaacagggaccccctggactcaagggtgctaagggggagccgggcagcaatggtgaccaaggtcccaaaggagacaggggtgtgccaggcatcaaaggagaccggggagagcctggaccgaggggtcaggacggcaacccgggtctaccaggagagcgtggtatggctgggcctgaagggaagccgggtctgcagggtccaagaggcccccctggcccagtgggtggtcatggagaccctggaccacctggtgccccgggtcttgctggccctgcaggaccccaaggaccttctggcctgaagggggagcctggagagacaggacctccaggacggggcctgactggacctactggagctgtgggacttcctggaccccccggcccttcaggccttgtgggtccacaggggtctccaggtttgcctggacaagtgggggagacagggaagccgggagccccaggtcgagatggtgccagtggaaaagatggagacagagggagccctggtgtgccagggtcaccaggtctgcctggccctgtcggacctaaaggagaacctggccccacgggggcccctggacaggctgtggtcgggctccctggagcaaagggagagaagggagcccctggaggccttgctggagacctggtgggtgagccgggagccaaaggtgaccgaggactgccagggccgcgaggcgagaagggtgaagctggccgtgcaggggagcccggagaccctggggaagatggtcagaaaggggctccaggacccaaaggtttcaagggtgacccaggagtcggggtcccgggctcccctgggcctcctggccctccaggtgtgaagggagatctgggcctccctggcctgcccggtgctcctggtgttgttgggttcccgggtcagacaggccctcgaggagagatgggtcagccaggccctagtggagagcggggtctggcaggccccccagggagagaaggaatcccaggacccctggggccacctggaccaccggggtcagtgggaccacctggggcctctggactcaaaggagacaagggagaccctggagtagggctgcctgggccccgaggcgagcgtggggagccaggcatccggggtgaagatggccgccccggccaggagggaccccgaggactcacggggccccctggcagcaggggagagcgtggggagaagggtgatgttgggagtgcaggactaaagggtgacaagggagactcagctgtgatcctggggcctccaggcccacggggtgccaagggggacatgggtgaacgagggcctcggggcttggatggtgacaaaggacctcggggagacaatggggaccctggtgacaagggcagcaagggagagcctggtgacaagggctcagccgggttgccaggactgcgtggactcctgggaccccagggtcaacctggtgcagcagggatccctggtgacccgggatccccaggaaaggatggagtgcctggtatccgaggagaaaaaggagatgttggcttcatgggtccccggggcctcaagggtgaacggggagtgaagggagcctgtggccttgatggagagaagggagacaagggagaagctggtcccccaggccgccccgggctggcaggacacaaaggagagatgggggagcctggtgtgccgggccagtcgggggcccctggcaaggagggcctgatcggtcccaagggtgaccgaggctttgacgggcagccaggccccaagggtgaccagggcgagaaaggggagcggggaaccccaggaattgggggcttcccaggccccagtggaaatgatggctctgctggtcccccagggccacctggcagtgttggtcccagaggccccgaaggacttcagggccagaagggtgagcgaggtccccccggagagagagtggtgggggctcctggggtccctggagctcctggcgagagaggggagcaggggcggccagggcctgccggtcctcgaggcgagaagggagaagctgcactgacggaggatgacatccggggctttgtgcgccaagagatgagtcagcactgtgcctgccagggccagttcatcgcatctggatcacgacccctccctagttatgctgcagacactgccggctcccagctccatgctgtgcctgtgctccgcgtctctcatgcagaggaggaagagcgggtaccccctgaggatgatgagtactctgaatactccgagtattctgtggaggagtaccaggaccctgaagctccttgggatagtgatgacccctgttccctgccactggatgagggctcctgcactgcctacaccctgcgctggtaccatcgggctgtgacaggcagcacagaggcctgtcacccttttgtctatggtggctgtggagggaatgccaaccgttttgggacccgtgaggcctgcgagcgccgctgcccaccccgggtggtccagagccaggggacaggtactgcccaggactga

SEQ ID NO: 2 - protein, 2944 aa, Homo sapiens
MTLRLLVAALCAGILAEAPRVRAQHRERVTCTRLYAADIVFLLDGSSSIGRSNFREVRSFLEGLVLPFSGAASAQGVRFATVQYSDDPRTEFGLDALGSGGDVIRAIRELSYKGGNTRTGAAILHVADHVFLPQLARPGVPKVCILITDGKSQDLVDTAAQRLKGQGVKLFAVGIKNADPEELKRVASQPTSDFFFFVNDFSILRTLLPLVSRRVCTTAGGVPVTRPPDDSTSAPRDLVLSEPSSQSLRVQWTAASGPVTGYKVQYTPLTGLGQPLPSERQEVNVPAGETSVRLRGLRPLTEYQVTVIALYANSIGEAVSGTARTTALEGPELTIQNTTAHSLLVAWRSVPGATGYRVTWRVLSGGPTQQQELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATELPGQRVRVSWSPVPGATQYRIIVRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQKGEPGEMGLRGQVGPPGDPGLPGRTGAPGPQGPPGSATAKGERGFPGADGRPGSPGRAGNPGTPGAPGLKGSPGLPGPRGDPGERGPRGPKGEPGAPGQVIGGEGPGLPGRKGDPGPSGPPGPRGPLGDPGPRGPPGLPGTAMKGDKGDRGERGPPGPGEGGIAPGEPGLPGLPGSPGPQGPVGPPGKKGEKGDSEDGAPGLPGQPGSPGEQGPRGPPGAIGPKGDRGFPGPLGEAGEKGERGPPGPAGSRGLPGVAGRPGAKGPEGPPGPTGRQGEKGEPGRPGDPAVVGPAVAGPKGEKGDVGPAGPRGATGVQGERGPPGLVLPGDPGPKGDPGDRGPIGLTGRAGPPGDSGPPGEKGDPGRPGPPGPVGPRGRDGEVGEKGDEGPPGDPGLPGKAGERGLRGAPGVRGPVGEKGDQGDPGEDGRNGSPGSSGPKGDRGEPGPPGPPGRLVDTGPGAREKGEPGDRGQEGPRGPKGDPGLPGAPGERGIEGFRGPPGPQGDPGVRGPAGEKGDRGPPGLDGRSGLDGKPGAAGPSGPNGAAGKAGDPGRDGLPGLRGEQGLPGPSGPPGLPGKPGEDGKPGLNGKNGEPGDPGEDGRKGEKGDSGASGREGRDGPKGERGAPGILGPQGPPGLPGPVGPPGQGFPGVPGGTGPKGDRGETGSKGEQGLPGERGLRGEPGSVPNVDRLLETAGIKASALREIVETWDESSGSFLPVPERRRGPKGDSGEQGPPGKEGPIGFPGERGLKGDRGDPGPQGPPGLALGERGPPGPSGLAGEPGKPGIPGLPGRAGGVGEAGRPGERGERGEKGERGEQGRDGPPGLPGTPGPPGPPGPKVSVDEPGPGLSGEQGPPGLKGAKGEPGSNGDQGPKGDRGVPGIKGDRGEPGPRGQDGNPGLPGERGMAGPEGKPGLQGPRGPPGPVGGHGDPGPPGAPGLAGPAGPQGPSGLKGEPGETGPPGRGLTGPTGAVGLPGPPGPSGLVGPQGSPGLPGQVGETGKPGAPGRDGASGKDGDRGSPGVPGSPGLPGPVGPKGEPGPTGAPGQAVVGLPGAKGEKGAPGGLAGDLVGEPGAKGDRGLPGPRGEKGEAGRAGEPGDPGEDGQKGAPGPKGFKGDPGVGVPGSPGPPGPPGVKGDLGLPGLPGAPGVVGFPGQTGPRGEMGQPGPSGERGLAGPPGREGIPGPLGPPGPPGSVGPPGASGLKGDKGDPGVGLPGPRGERGEPGIRGEDGRPGQEGPRGLTGPPGSRGERGEKGDVGSAGLKGDKGDSAVILGPPGPRGAKGDMGERGPRGLDGDKGPRGDNGDPGDKGSKGEPGDKGSAGLPGLRGLLGPQGQPGAAGIPGDPGSPGKDGVPGIRGEKGDVGFMGPRGLKGERGVKGACGLDGEKGDKGEAGPPGRPGLAGHKGEMGEPGVPGQSGAPGKEGLIGPKGDRGFDGQPGPKGDQGEKGERGTPGIGGFPGPSGNDGSAGPPGPPGSVGPRGPEGLQGQKGERGPPGERVVGAPGVPGAPGERGEQGRPGPAGPRGEKGEAALTEDDIRGFVRQEMSQHCACQGQFIASGSRPLPSYAADTAGSQLHAVPVLRVSHAEEEERVPPEDDEYSEYSEYSVEEYQDPEAPWDSDDPCSLPLDEGSCTAYTLRWYHRAVTGSTEACHPFVYGGCGGNANRFGTREACERRCPPRVVQSQGTGTAQD

SEQ ID NO: 3 - DNA, 4494 bp, Homo sapiens
atggatgtaaccaagaaaaacaaacgagatggaactgaagtcactgagagaattgtcactgaaacagtaaccacaagacttacatccttaccaccaaaaggcgggaccagcaatggctatgctaaaacagcctctcttggtggagggagccggctggagaaacaaagcctgactcatggcagcagcggctacataaactcaactggaagcacacgaggccatgcctccacctctagttacaggagggctcactcacctgcctccactctgcccaactccccaggctcaacctttgaaaggaaaactcacgttacccgccatgcgtatgaagggagctccagtggcaactcttctccggagtaccctcggaaggaatttgcatcttcttcaaccagaggacggagtcaaacacgagagagtgaaattcgagttcgactgcagagtgcgtccccatccacccgatggacagaattggatgatgttaagcgtttgctcaaggggagtcgatcggcaagtgtgagccccacccggaattcctccaacacactccccatccccaagaaaggcactgtggagaccaaaattgtgacagcgagctcccagtcggtgtcaggcacctacgatgcaacgatcctggatgccaaccttccctcccatgtgtggtcctccaccctgcccgcggggtcctccatggggacctatcacaacaacatgacaacccagagctcatccctcctcaacaccaatgcctactctgcgggatcagtcttcggagttccaaacaacatggcgtcctgctcacccactttgcaccctggactcagcacatcctcctcagtgtttggcatgcagaacaatctggcccccagcttgaccaccctgtcccatggcaccaccaccacttccacagcatatggggtgaagaaaaacatgccccagagtcctgcggctgtgaacactggcgtttccacctccgccgcctgcaccacaagtgtgcagagcgatgaccttttgcacaaggactgcaagttcctgatcctagagaaagacaacacacctgccaagaaggagatggagctgctcatcatgaccaaggacagcgggaaggtctttacagcctcccctgccagcatcgctgcaacttctttttcagaagacaccctaaaaaaagaaaagcaagctgcctacaatgctgactcaggcctaaaagccgaagctaatggagacctgaagactgtgtccacaaagggcaagaccaccactgcagatatccacagctacggcagcagtggtggtggtggcagtggaggaggtggcggtgttggtggcgctggcggcggcccttggggaccagcgccagcctggtgcccctgcggctcctgctgcagctggtggaagtggctgctgggcctgctgctcacctggctgctactcctggggctgctcttcggcctcattgctctggcggaggaggtgaggaagctgaaggcgcgtgtggatgagctggagaggatcaggaggagcatactgccctatggggacagcatggatagaatagaaaaggaccgcctccagggcatggcacccgcggcgggagcagacctggacaaaattgggctgcacagtgacagccaggaggagctctggatgttcgtgaggaagaagctaatgatggaacaggaaaatggaaatctccgaggaagccctggccctaaaggtgacatgggaagtccaggccctaaaggagatcgagggttccctgggactccaggtatccctgggcccttgggccacccaggtccacaaggaccaaagggtcaaaaaggcagcgtgggagatcctggcatggaaggccccatgggccagagagggcgagaaggccccatgggacctcgtggtgaggcagggcctcctggatctggagagaaaggggaaagaggggctgctggtgaaccaggtcctcatggcccacctggtgtcccaggttctgtgggtcccaaaggttccagcggctctcctggcccacagggccctccaggtcctgtaggtctccaagggctccgaggtgaagtaggacttcctggtgtcaaaggtgacaaaggaccaatgggaccaccaggacccaaaggtgaccagggtgagaaaggacctcgaggcctcacaggcgagcctggcatgagaggtttgcctggtgctgttggtgagcccggggctaaaggagcaatgggtcctgctggcccagacggacaccaaggcccaagaggtgaacaaggtcttactgggatgcctggaatccgtggcccaccaggaccttctggagacccaggaaagccaggtctcacaggaccccagggacctcagggacttcccggtacccctggccgaccaggaataaaaggtgaaccaggagctccaggcaagatcgtgacttcggaggggtcatcgatgctcactgtcccaggccccccaggacctcctggagccatgggacccccaggacctccaggtgccccaggccctgccggcccagctggtctcccaggacatcaagaagttcttaatttacaaggtcccccaggcccacccggcccacgcgggccaccagggccttccattccaggcccaccaggaccccgaggcccaccaggggagggtttgccaggcccaccaggcccaccaggatcgttcctgtccaactcagaaaccttcctctccggccccccaggcccacctggccccccaggtcccaagggagaccaaggtcccccaggccccagaggacaccaaggcgagcaaggcctcccaggtttctcaacctcagggtccagttctttcggactcaaccttcagggaccaccaggcccacctggcccccagggacccaaaggtgacaaaggtgatccaggtgttccaggggctcttggcattcctagtggtccttctgaagggggatcatcaagtaccatgtacgtgtcaggcccgccagggccccctgggccccctgggcctccgggctctatcagcagctctggccaggagattcagcagtacatctctgagtacatgcagagtgacagtattagatcttacctatccggagttcagggtcccccaggcccacctggtcccccaggacctgtcaccaccatcacaggcgagactttcgactactcagagctggcaagccacgttgtgagctacttacggacttcggggtacggtgtcagcttgttctcgtcctccatctcttctgaagacattctggctgtgctgcagcgggatgacgtgcgtcagtacctacgtcagtacttgatgggccctcggggtccgccagggccaccaggagccagtggagatgggtccctcctgtctttggactatgcagagctgagtagtcgcattctcagctacatgtcgagttctgggatcagcattgggcttcctggtcccccggggccccctggcttgccgggaacctcctatgaggagctcctctccttgctgcgagggtctgaattcagaggcatcgttggacccccaggtcccccgggtccaccagggatcccaggcaatgtgtggtccagcatcagcgtggaggacctctcgtcttacttacatactgccggcttgtcattcatcccaggccctccaggacctcctggtcccccagggcctcgagggcccccgggtgtctcaggagccctggcaacctatgcagctgaaaacagcgacagcttccggagcgagctgatcagctacctcacaagtcctgatgtgcgcagcttcattgttggccccccaggccctcctgggccgcagggaccccctggggacagccgcctcctgtccacggatgcctcccacagtcggggtagcagctcctcctcacacagctcatctgtcaggcggggcagctcctacagctcttccatgagcacaggaggaggtggtgcaggctccctgggtgcaggcggtgcctttggtgaagctgcaggagacaggggtccctatggcactgacatcggcccaggcggaggctatggggcagcagcagaaggcggcatgtatgctggcaatggcggactattgggagctgactttgctggagatctggattacaatgagctggctgtgagggtgtcagagagcatgcagcgtcagggcctactgcaagggatggcctacactgtccagggcccaccaggccagcctgggccacaggggccacccggcatcagcaaggtcttctctgcctacagcaacgtgactgcggacctcatggacttcttccaaacttatggagccattcaaggaccccctgggcaaaaaggagagatgggcactccaggacccaaaggtgacaggggccctgctgggccaccaggtcatcctgggccacctggccctcgaggacacaagggagaaaaaggagacaaaggtgaccaagtctatgctgggcggagaaggagaagaagtattgctgtcaagccgtga

SEQ ID NO: 4 - protein, 1497 aa, Homo sapiens
MDVTKKNKRDGTEVTERIVTETVTTRLTSLPPKGGTSNGYAKTASLGGGSRLEKQSLTHGSSGYINSTGSTRGHASTSSYRRAHSPASTLPNSPGSTFERKTHVTRHAYEGSSSGNSSPEYPRKEFASSSTRGRSQTRESEIRVRLQSASPSTRWTELDDVKRLLKGSRSASVSPTRNSSNTLPIPKKGTVETKIVTASSQSVSGTYDATILDANLPSHVWSSTLPAGSSMGTYHNNMTTQSSSLLNTNAYSAGSVFGVPNNMASCSPTLHPGLSTSSSVFGMQNNLAPSLTTLSHGTTTTSTAYGVKKNMPQSPAAVNTGVSTSAACTTSVQSDDLLHKDCKFLILEKDNTPAKKEMELLIMTKDSGKVFTASPASIAATSFSEDTLKKEKQAAYNADSGLKAEANGDLKTVSTKGKTTTADIHSYGSSGGGGSGGGGGVGGAGGGPWGPAPAWCPCGSCCSWWKWLLGLLLTWLLLLGLLFGLIALAEEVRKLKARVDELERIRRSILPYGDSMDRIEKDRLQGMAPAAGADLDKIGLHSDSQEELWMFVRKKLMMEQENGNLRGSPGPKGDMGSPGPKGDRGFPGTPGIPGPLGHPGPQGPKGQKGSVGDPGMEGPMGQRGREGPMGPRGEAGPPGSGEKGERGAAGEPGPHGPPGVPGSVGPKGSSGSPGPQGPPGPVGLQGLRGEVGLPGVKGDKGPMGPPGPKGDQGEKGPRGLTGEPGMRGLPGAVGEPGAKGAMGPAGPDGHQGPRGEQGLTGMPGIRGPPGPSGDPGKPGLTGPQGPQGLPGTPGRPGIKGEPGAPGKIVTSEGSSMLTVPGPPGPPGAMGPPGPPGAPGPAGPAGLPGHQEVLNLQGPPGPPGPRGPPGPSIPGPPGPRGPPGEGLPGPPGPPGSFLSNSETFLSGPPGPPGPPGPKGDQGPPGPRGHQGEQGLPGFSTSGSSSFGLNLQGPPGPPGPQGPKGDKGDPGVPGALGIPSGPSEGGSSSTMYVSGPPGPPGPPGPPGSISSSGQEIQQYISEYMQSDSIRSYLSGVQGPPGPPGPPGPVTTITGETFDYSELASHVVSYLRTSGYGVSLFSSSISSEDILAVLQRDDVRQYLRQYLMGPRGPPGPPGASGDGSLLSLDYAELSSRILSYMSSSGISIGLPGPPGPPGLPGTSYEELLSLLRGSEFRGIVGPPGPPGPPGIPGNVWSSISVEDLSSYLHTAGLSFIPGPPGPPGPPGPRGPPGVSGALATYAAENSDSFRSELISYLTSPDVRSFIVGPPGPPGPQGPPGDSRLLSTDASHSRGSSSSSHSSSVRRGSSYSSSMSTGGGGAGSLGAGGAFGEAAGDRGPYGTDIGPGGGYGAAAEGGMYAGNGGLLGADFAGDLDYNELAVRVSESMQRQGLLQGMAYTVQGPPGQPGPQGPPGISKVFSAYSNVTADLMDFFQTYGAIQGPPGQKGEMGTPGPKGDRGPAGPPGHPGPPGPRGHKGEKGDKGDQVYAGRRRRRSIAVKP

SEQ ID NO: 5 - DNA, 3519 bp, Homo sapiens
atgagaccattcttcctcttgtgttttgccctgcctggcctcctgcatgcccaacaagcctgctcccgtggggcctgctatccacctgttggggacctgcttgttgggaggacccggtttctccgagcttcatctacctgtggactgaccaagcctgagacctactgcacccagtatggcgagtggcagatgaaatgctgcaagtgtgactccaggcagcctcacaactactacagtcaccgagtagagaatgtggcttcatcctccggccccatgcgctggtggcagtcacagaatgatgtgaaccctgtctctctgcagctggacctggacaggagattccagcttcaagaagtcatgatggagttccaggggcccatgcccgccggcatgctgattgagcgctcctcagacttcggtaagacctggcgagtgtaccagtacctggctgccgactgcacctccaccttccctcgggtccgccagggtcggcctcagagctggcaggatgttcggtgccagtccctgcctcagaggcctaatgcacgcctaaatggggggaaggtccaacttaaccttatggatttagtgtctgggattccagcaactcaaagtcaaaaaattcaagaggtgggggagatcacaaacttgagagtcaatttcaccaggctggcccctgtgccccaaaggggctaccaccctcccagcgcctactatgctgtgtcccagctccgtctgcaggggagctgcttctgtcacggccatgctgatcgctgcgcacccaagcctggggcctctgcaggcccctccaccgctgtgcaggtccacgatgtctgtgtctgccagcacaacactgccggcccaaattgtgagcgctgtgcacccttctacaacaaccggccctggagaccggcggagggccaggacgcccatgaatgccaaaggtgcgactgcaatgggcactcagagacatgtcactttgaccccgctgtgtttgccgccagccagggggcatatggaggtgtgtgtgacaattgccgggaccacaccgaaggcaagaactgtgagcggtgtcagctgcactatttccggaaccggcgcccgggagcttccattcaggagacctgcatctcctgcgagtgtgatccggatggggcagtgccaggggctccctgtgacccagtgaccgggcagtgtgtgtgcaaggagcatgtgcagggagagcgctgtgacctatgcaagccgggcttcactggactcacctacgccaacccgcagggctgccaccgctgtgactgcaacatcctggggtcccggagggacatgccgtgtgacgaggagagtgggcgctgcctttgtctgcccaacgtggtgggtcccaaatgtgaccagtgtgctccctaccactggaagctggccagtggccagggctgtgaaccgtgtgcctgcgacccgcacaactccctcagcccacagtgcaaccagttcacagggcagtgcccctgtcgggaaggctttggtggcctgatgtgcagcgctgcagccatccgccagtgtccagaccggacctatggagacgtggccacaggatgccgagcctgtgactgtgatttccggggaacagagggcccgggctgcgacaaggcatcaggccgctgcctctgccgccctggcttgaccgggccccgctgtgaccagtgccagcgaggctactgtaatcgctacccggtgtgcgtggcctgccacccttgcttccagacctatgatgcggacctccgggagcaggccctgcgctttggtagactccgcaatgccaccgccagcctgtggtcagggcctgggctggaggaccgtggcctggcctcccggatcctagatgcaaagagtaagattgagcagatccgagcagttctcagcagccccgcagtcacagagcaggaggtggctcaggtggccagtgccatcctctccctcaggcgaactctccagggcctgcagctggatctgcccctggaggaggagacgttgtcccttccgagagacctggagagtcttgacagaagcttcaatggtctccttactatgtatcagaggaagagggagcagtttgaaaaaataagcagtgctgatccttcaggagccttccggatgctgagcacagcctacgagcagtcagcccaggctgctcagcaggtctccgacagctcgcgccttttggaccagctcagggacagccggagagaggcagagaggctggtgcggcaggcgggaggaggaggaggcaccggcagccccaagcttgtggccctgaggctggagatgtcttcgttgcctgacctgacacccaccttcaacaagctctgtggcaactccaggcagatggcttgcaccccaatatcatgccctggtgagctatgtccccaagacaatggcacagcctgtggctcccgctgcaggggtgtccttcccagggccggtggggccttcttgatggcggggcaggtggctgagcagctgcggggcttcaatgcccagctccagcggaccaggcagatgattagggcagccgaggaatctgcctcacagattcaatccagtgcccagcgcttggagacccaggtgagcgccagccgctcccagatggaggaagatgtcagacgcacacggctcctaatccagcaggtccgggacttcctaacagaccccgacactgatgcagccactatccaggaggtcagcgaggccgtgctggccctgtggctgcccacagactcagctactgttctgcagaagatgaatgagatccaggccattgcagccaggctccccaacgtggacttggtgctgtcccagaccaagcaggacattgcgcgtgcccgccggttgcaggctgaggctgaggaagccaggagccgagcccatgcagtggagggccaggtggaagatgtggttgggaacctgcggcaggggacagtggcactgcaggaagctcaggacaccatgcaaggcaccagccgctcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcagcggacctgacaggactggagaagcgtgtggagcagatccgtgaccacatcaatgggcgcgtgctctactatgccacctgcaagtga

SEQ ID NO: 6 - protein, 1172 aa, Homo sapiens [sequence omitted]

SEQ ID NO: 7 - DNA, 9834 bp, Homo sapiens
atggcggcggccgcgcggcctcggggtcgggcactggggccagtactgccgccgacgccgctgctcctgctggtactgcgggtgctgccagcctgcggggcgaccgctcgggatcccggggccgcggccgggctcagccttcacccgacttacttcaacctggccgaggcggcgaggatttgggccaccgccacctgcggggagaggggacccggcgaggggaggccccagcccgagctctactgcaagttggtcgggggccccaccgccccaggcagcggccacaccatccagggccagttctgtgactattgcaattctgaagaccccaggaaagcacatcctgtcaccaatgccatcgatggatctgaacgttggtggcaaagccctcccctgtcctcaggcacacagtacaacagagtcaacctcaccttggatctggggcagctcttccatgtggcctatattttaatcaaatttgcaaattctcctcgccctgatctttgggtcttggaaagatctgtagactttggaagcacctactcaccatggcaatattttgctcattctaaagtagactgtttaaaagaatttgggcgggaggcaaatatggctgtcacccgggatgatgatgtactttgtgttactgaatattcccgtattgtacctttggaaaatggtgaggttgtggtgtccttgataaacggtcgtccaggtgcaaaaaattttactttctctcacaccctgagggagtttaccaaggcaacaaacatccgcttgcgttttcttagaaccaatacgcttcttggacacctcatctccaaagcccagcgagatccaactgtcactcggcggtattattacagcataaaggacatcagcattggtgggcagtgtgtttgcaatggccatgctgaagtgtgcaatataaacaatcctgaaaaactgtttcggtgtgaatgccagcaccacacctgtggggagacgtgtgatcgctgctgcacagggtacaatcagaggcgctggcggcccgccgcttgggagcagagccacgagtgtgaagcatgcaactgccacggccatgccagcaactgttactatgatccagatgttgagcggcagcaggcaagcttgaatacccagggcatctatgctggtggaggggtctgcattaactgtcagcacaacacagctggagtaaactgtgaacagtgtgctaagggctattaccgcccttatggggttccagtggatgcccctgatggctgcatcccctgcagctgtgaccctgagcatgcggatggctgtgaacagggttcaggccgctgtcactgcaagccaaatttccacggagacaactgtgagaagtgtgcaattggatactacaatttcccattttgcttgagaattcccatttttcctgtttctacaccaagttcagaagatccagtagctggagatataaaagggtgtgactgtaatctggaaggtgttctccctgaaatatgtgatgcccacggacggtgcctgtgccgccctggggttgagggccctcgatgtgatacctgccgctctggtttctactcattccctatttgccaagcctgctggtgttcagcccttggatcctaccagatgccctgcagctcagtgactggacagtgtgaatgtcggccaggagttacaggacagcggtgtgacaggtgtctctcaggagcttatgatttcccccactgccaaggttccagcagtgcttgtgacccagctggtaccatcaactccaatttggggtattgccaatgcaagcttcatgttgaaggtcctacttgtagccgctgcaaactgttatattggaatctggacaaagaaaaccccagtggatgttcagaatgcaagtgccataaggcgggaacagtgagtggaactggagagtgtaggcagggagatggtgactgtcactgcaagtcccatgtgggtggcgattcctgcgacacctgtgaagatggatattttgctttggaaaagagcaattactttgggtgtcaagggtgtcagtgtgacattggtggggcattgtcctccatgtgcagtgggccctcgggagtgtgccagtgccgagagcatgtcgtgggaaaggtgtgccagcggcctgaaaacaactactatttcccagatttgcatcatatgaagtatgagattgaagacggcagcacacctaatgggagagaccttcgatttggatttgatccgctggcatttcctgagtttagctggagaggatatgcccaaatgacctcagtacagaatgatgtaagaataacattgaatgtagggaagtcaagtggctccttgtttcgtgttattctgagatacgttaaccctggaactgaagcagtatctggccatataactatttatccatcctggggtgctgctcaaagcaaagagatcatcttcctgccgagtaaggagccagcctttgtcactgtccctggaaatggttttgcagacccattttcaatcacaccaggaatatgggttgcttgtattaaggcagaaggagtccttctggattacctggtgctgctccccagggactactatgaagcctctgtactgcagctgccagtcacagaaccatgtgcctacgcaggacctccccaagaaaattgcttactctaccagcatttgccagtgaccagattcccctgtaccctggcttgtgaggccagacacttcctgcttgatggggagccaagacccgtggcagtgaggcagcccacacctgcacaccctgtcatggtggacctcagcgggagagaggtggaattgcatctgcggctgcgcatcccacaggttggccactacgtggttgtggtcgagtattccacggaggcagctcagctgtttgtggttgatgtgaatgtgaagagctccgggtctgttctggcaggccaggtgaacatttacagctgcaactacagtgttctctgccggagtgctgtgattgatcacatgagccgcatcgccatgtatgagctattggcagatgcagacattcagctcaagggacacatggcccgattccttctgcatcaagtttgtatcatacctattgaagaattctcagctgagtatgtgagaccacaagtccactgcattgccagttatgggcgatttgtcaatcaaagtgccacctgtgtctccttggcccatgaaactcctccaacagcattaattttggatgttctaagtggcaggcctttccctcacctgccccagcagtcgtcaccttctgttgatgttcttcctggggtcaccttgaaggcaccgcagaatcaagtgaccctgagaggacgtgtaccacacctgggccgatacgtctttgtcatccatttttaccaagcagcgcacccgacgtttcccgcgcaggtgtcggtggatggcgggtggccacgggcaggctccttccatgcctctttttgcccccatgtgcttggctgccgggatcaagtgattgccgaaggccagattgagtttgacatctcagagcctgaagtggccgcaactgtgaaggttccagaaggaaagtccttggttttggtccgtgttctagtggtgcctgcagaaaactatgactaccaaatacttcacaaaaaatccatggacaagtcactcgagtttatcaccaattgtggaaaaaacagcttttaccttgacccccagacagcctccagattctgtaagaattccgccaggtccctggtggccttttaccacaagggcgccctgccttgtgagtgccaccccactggggccaccggccctcactgcagccctgagggtgggcagtgcccatgccagcccaacgtcatcgggcggcagtgcacccgctgtgcaacaggccactacggattcccacgctgcaagccgtgcagctgtggtcggcgcctttgtgaagagatgacggggcagtgccgctgccctccccgcacggtcaggccccagtgtgaggtgtgtgagacacactcattcagcttccaccccatggccggctgcgaaggctgcaactgttccaggaggggcaccatcgaggctgccatgccggagtgtgaccgggacagcgggcagtgcagatgcaagcccagaatcacagggcggcagtgtgaccgatgtgcttccgggttttaccgctttcctgagtgtgttccctgcaattgcaacagagatgggactgagccaggagtgtgtgacccagggaccggggcttgcctctgcaaggaaaatgtagaaggcacagagtgtaatgtgtgtcgagaaggctcattccatttggacccagccaatctcaagggttgtaccagctgtttctgttttggagtaaataatcaatgtcacagctcacataagcgaaggactaagtttgtggatatgctgggctggcacctggagacagcagacagagtggacatccctgtctctttcaacccaggcagcaacagtatggtggcggatctccaggagctgcccgcaaccatccacagcgcgtcctgggtcgcacccacctcctacctgggggacaaggtttcttcatatggtggttacctcacttaccaagccaagtcctttggcttgcctggcgacatggttcttctggaaaagaagccggatgtacagctcactggtcagcacatgtccatcatctatgaggagacaaacaccccacggccagaccggctgcatcatggacgagtgcacgtggtcgagggaaacttcagacatgccagcagccgtgccccagtgtctagggaggagctgatgacagtgctgtctagactggcagatgtgcgcatccaaggcctctacttcacagagactcaaaggctcaccctgagcgaggtggggctagaggaagcctctgacacaggaagtgggcgcatagcacttgctgtggaaatctgtgcctgcccccctgcctacgctggtgactcttgtcagggttgtagccctggatactatcgggatcataaaggcttgtataccggacggtgtgttccctgcaattgcaacggacattcaaatcaatgccaggatggctcaggcatatgtgttaactgtcagcacaacaccgcgggagagcactgtgaacgctgccaggagggctactatggcaacgccgtccacggatcctgcagggcctgcccatgtcctcacactaacagctttgccactggctgtgtggtgaatgggggagacgtgcggtgctcctgcaaagctgggtacacaggaacacagtgtgaaaggtgtgcaccgggatatttcgggaatccccagaaattcggaggtagctgccaaccatgcagttgtaacagcaatggccagctgggcagctgtcatcccctgactggagactgcataaaccaagaacccaaagatagcagccctgcagaagaatgtgatgattgcgacagctgtgtgatgaccctcctgaacgacctggccaccatgggcgagcagctccgcctggtcaagtctcagctgcagggcctgagtgccagcgcagggcttctggagcagatgaggcacatggagacccaggccaaggacctgaggaatcagttgctcaactaccgttctgccatttcaaatcatggatcaaaaatagaaggcctggaaagagaactgactgatttgaatcaagaatttgagactttgcaagaaaaggctcaagtaaattccagaaaagcacaaacattaaacaacaatgttaatcgggcaacacaaagcgcaaaagaactggatgtgaagattaaaaatgtcatccggaatgtgcacatgctgaaccggataaggacctggcagaaaacccaccagggggagaacaatgggcttgctaacagtatccgggattctttaaatgaatacgaagccaaactcagtgaccttcgtgctcggctgcaggaggcagctgcccaagccaagcaggcaaatggcttgaaccaagaaaacgagagagctttgggagccattcagagacaagtgaaagaaataaattccctgcagagtgatttcaccaagtatctaaccactgcagactcatctttgttgcaaaccaacattgcgctgcagctgatggagaaaagccagaaggaatatgaaaaattagctgccagtttaaatgaagcaagacaagaactaagtgacaaagtaagagaactttccagatctgctggcaaaacatcccttgtggaggaggcagaaaagcacgcgcggtccttacaagagctggcaaagcagctggaagagatcaagagaaacgccagcggggatgagctggtgcgctgtgctgtggatgccgccaccgcctacgagaacatcctcaatgccatcaaagcggccgaggacgcagccaacagggctgccagtgcatctgaatctgccctccagacagtgataaaggaagatctgccaagaaaagctaaaaccctgagttccaacagtgataaactgttaaatgaagccaagatgacacaaaagaagctaaagcaagaagtcagtccagctctcaacaacctacagcaaaccctgaatattgtgacagttcagaaagaagtgatagacaccaatctcacaactctccgagatggtcttcatgggatacagagaggtgatattgatgctatgatcagtagtgcaaagagcatggtcagaaaggccaacgacatcacagatgaggttctggatgggctcaaccccatccagacagatgtggaaagaattaaggacacctatgggaggacacagaacgaagacttcaaaaaggctctgactgatgcagataactcggtgaataagttaaccaacaaactacctgatctttggcgcaagattgaaagtatcaaccaacagctgttgcccttgggaaacatctctgacaacatggacagaatacgagaactaattcagcaggccagagatgctgccagtaaggttgctgtccccatgaggttcaatggtaaatctggagtcgaagtccgactgccaaatgacctggaagatttgaaaggatatacatctctgtccttgtttctccaaaggcccaactcaagagaaaatgggggtactgagaatatgtttgtgatgtaccttggaaataaagatgcctcccgggactacatcggcatggcagttgtggatggccagctcacctgtgtctacaacctgggggaccgtgaggctgaactccaagtggaccagatcttgaccaagagtgagactaaggaggcagttatggatcgggtgaaatttcagagaatttatcagtttgcaaggcttaattacaccaaaggagccacatccagtaaaccagaaacacccggagtctatgacatggatggtagaaatagcaatacactccttaatttggatcctgaaaatgttgtattttatgttggaggttacccacctgattttaaacttcccagtcgactaagtttccctccatacaaaggttgtattgaattagatgacctcaatgaaaatgttctgagcttgtacaacttcaaaaaaacattcaatctcaacacaactgaagtggagccttgtagaaggaggaaggaagagtcagacaaaaattattttgaaggtacgggctatgctcgagttccaactcaaccacatgctcccatcccaacctttggacagacaattcagaccaccgtggatagaggcttgctgttctttgcagaaaacggggatcgcttcatatctctaaatatagaagatggcaagctcatggtgagatacaaactgaattcagagctaccaaaagagagaggagttggagacgccataaacaacggcagagaccattcgattcagatcaaaattggaaaactccaaaagcgtatgtggataaatgtggacgttcaaaacactataattgatggtgaagtatttgatttcagcacatattatctgggaggaattccaattgcaatcagggaaagatttaacatttctacgcctgctttccgaggctgcatgaaaaatttgaagaaaaccagtggtgtcgttagattgaatgatactgtgggagtaaccaaaaagtgctcggaagactggaagcttgtgcgatctgcctcattctccagaggaggacaattgagtttcactgatttgggcttaccacctactgaccacctccaggcctcatttggatttcagacctttcaacccagtggcatattattagatcatcagacatggacaaggaacctgcaggtcactctggaagatggttacattgaattgagcaccagcgatagcggcggcccaatttttaaatctccacagacgtatatggatggtttactgcattatgtatctgtaataagcgacaactctggactacggcttctcatcgatgaccagcttctgagaaatagcaaaaggctaaaacacatttcaagttcccggcagtctctgcgtctgggcgggagcaattttgagggttgtattagcaatgtttttgtccagaggttatcactgagtcctgaagtcctagatttgaccagtaactctctcaagagagatgtgtccctgggaggctgcagtttaaacaaaccaccttttctaatgttgcttaaaggttctaccaggtttaacaagaccaagacttttcgtatcaaccagctgttgcaggacacaccagtggcctccccaaggagcgtgaaggtgtggcaagatgcttgctcaccacttcccaagacccaggccaatcatggagccctccagtttggggacattcccaccagccacttgctattcaagcttcctcaggagctgctgaaacccaggtcacagtttgctgtggacatgcagacaacatcctccagaggactggtgtttcacacgggcactaagaactcctttatggctctttatctttcaaaaggacgtctggtctttgcactggggacagatgggaaaaaattgaggatcaaaagcaaggagaaatgcaatgatgggaaatggcacacggtggtgtttggccatgatggggaaaaggggcgcttggttgtggatggactgagggcccgggagggaagtttgcctggaaactccaccatcagcatcagagcgccagtttacctgggatcacctccatcagggaaaccaaagagcctccccacaaacagctttgtgggatgcctgaagaactttcagctggattcaaaacccttgtataccccttcttcaagcttcggggtgtcttcctgcttgggtggtcctttggagaaaggcatttatttctctgaagaaggaggtcatgtcgtcttggctcactctgtattgttggggccagaatttaagcttgttttcagcatccgcccaagaagtctcactgggatcctaatacacatcggaagtcagcccgggaagcacttatgtgtttacctggaggcaggaaaggtcacggcctctatggacagtggggcaggtgggacctcaacgtcggtcacaccaaagcagtctctgtgtgatggacagtggcactcggtggcagtcaccataaaacaacacatcctgcacctggaactggacacagacagtagctacacagctggacagatccccttcccacctgccagcactcaagagccactacaccttggaggtgctccagccaatttgacgacactgaggatccctgtgtggaaatcattctttggctgtctgaggaatattcatgtcaatcacatccctgtccctgtcactgaagccttggaagtccaggggcctgtcagtctgaatggttgtcctgaccagtaa

SEQ ID NO: 8 - protein, 3333 aa, Homo sapiens
MAAAARPRGRALGPVLPPTPLLLLVLRVLPACGATARDPGAAAGLSLHPTYFNLAEAARIWATATCGERGPGEGRPQPELYCKLVGGPTAPGSGHTIQGQFCDYCNSEDPRKAHPVTNAIDGSERWWQSPPLSSGTQYNRVNLTLDLGQLFHVAYILIKFANSPRPDLWVLERSVDFGSTYSPWQYFAHSKVDCLKEFGREANMAVTRDDDVLCVTEYSRIVPLENGEVVVSLINGRPGAKNFTFSHTLREFTKATNIRLRFLRTNTLLGHLISKAQRDPTVTRRYYYSIKDISIGGQCVCNGHAEVCNINNPEKLFRCECQHHTCGETCDRCCTGYNQRRWRPAAWEQSHECEACNCHGHASNCYYDPDVERQQASLNTQGIYAGGGVCINCQHNTAGVNCEQCAKGYYRPYGVPVDAPDGCIPCSCDPEHADGCEQGSGRCHCKPNFHGDNCEKCAIGYYNFPFCLRIPIFPVSTPSSEDPVAGDIKGCDCNLEGVLPEICDAHGRCLCRPGVEGPRCDTCRSGFYSFPICQACWCSALGSYQMPCSSVTGQCECRPGVTGQRCDRCLSGAYDFPHCQGSSSACDPAGTINSNLGYCQCKLHVEGPTCSRCKLLYWNLDKENPSGCSECKCHKAGTVSGTGECRQGDGDCHCKSHVGGDSCDTCEDGYFALEKSNYFGCQGCQCDIGGALSSMCSGPSGVCQCREHVVGKVCQRPENNYYFPDLHHMKYEIEDGSTPNGRDLRFGFDPLAFPEFSWRGYAQMTSVQNDVRITLNVGKSSGSLFRVILRYVNPGTEAVSGHITIYPSWGAAQSKEIIFLPSKEPAFVTVPGNGFADPFSITPGIWVACIKAEGVLLDYLVLLPRDYYEASVLQLPVTEPCAYAGPPQENCLLYQHLPVTRFPCTLACEARHFLLDGEPRPVAVRQPTPAHPVMVDLSGREVELHLRLRIPQVGHYVVVVEYSTEAAQLFVVDVNVKSSGSVLAGQVNIYSCNYSVLCRSAVIDHMSRIAMYELLADADIQLKGHMARFLLHQVCIIPIEEFSAEYVRPQVHCIASYGRFVNQSATCVSLAHETPPTALILDVLSGRPFPHLPQQSSPSVDVLPGVTLKAPQNQVTLRGRVPHLGRYVFVIHFYQAAHPTFPAQVSVDGGWPRAGSFHASFCPHVLGCRDQVIAEGQIEFDISEPEVAATVKVPEGKSLVLVRVLVVPAENYDYQILHKKSMDKSLEFITNCGKNSFYLDPQTASRFCKNSARSLVAFYHKGALPCECHPTGATGPHCSPEGGQCPCQPNVIGRQCTRCATGHYGFPRCKPCSCGRRLCEEMTGQCRCPPRTVRPQCEVCETHSFSFHPMAGCEGCNCSRRGTIEAAMPECDRDSGQCRCKPRITGRQCDRCASGFYRFPECVPCNCNRDGTEPGVCDPGTGACLCKENVEGTECNVCREGSFHLDPANLKGCTSCFCFGVNNQCHSSHKRRTKFVDMLGWHLETADRVDIPVSFNPGSNSMVADLQELPATIHSASWVAPTSYLGDKVSSYGGYLTYQAKSFGLPGDMVLLEKKPDVQLTGQHMSIIYEETNTPRPDRLHHGRVHVVEGNFRHASSRAPVSREELMTVLSRLADVRIQGLYFTETQRLTLSEVGLEEASDTGSGRIALAVEICACPPAYAGDSCQGCSPGYYRDHKGLYTGRCVPCNCNGHSNQCQDGSGICVNCQHNTAGEHCERCQEGYYGNAVHGSCRACPCPHTNSFATGCVVNGGDVRCSCKAGYTGTQCERCAPGYFGNPQKFGGSCQPCSCNSNGQLGSCHPLTGDCINQEPKDSSPAEECDDCDSCVMTLLNDLATMGEQLRLVKSQLQGLSASAGLLEQMRHMETQAKDLRNQLLNYRSAISNHGSKIEGLERELTDLNQEFETLQEKAQVNSRKAQTLNNNVNRATQSAKELDVKIKNVIRNVHILLKQISGTDGEGNNVPSGDFSREWAEAQRMMRELRNRNFGKHLREAEADKRESQLLLNRIRTWQKTHQGENNGLANSIRDSLNEYEAKLSDLRARLQEAAAQAKQANGLNQENERALGAIQRQVKEINSLQSDFTKYLTTADSSLLQTNIALQLMEKSQKEYEKLAASLNEARQELSDKVRELSRSAGKTSLVEEAEKHARSLQELAKQLEEIKRNASGDELVRCAVDAATAYENILNAIKAAEDAANRAASASESALQTVIKEDLPRKAKTLSSNSDKLLNEAKMTQKKLKQEVSPALNNLQQTLNIVTVQKEVIDTNLTTLRDGLHGIQRGDIDAMISSAKSMVRKANDITDEVLDGLNPIQTDVERIKDTYGRTQNEDFKKALTDADNSVNKLTNKLPDLWRKIESINQQLLPLGNISDNMDRIRELIQQARDAASKVAVPMRFNGKSGVEVRLPNDLEDLKGYTSLSLFLQRPNSRENGGTENMFVMYLGNKDASRDYIGMAVVDGQLTCVYNLGDREAELQVDQILTKSETKEAVMDRVKFQRIYQFARLNYTKGATSSKPETPGVYDMDGRNSNTLLNLDPENVVFYVGGYPPDFKLPSRLSFPPYKGCIELDDLNENVLSLYNFKKTFNLNTTEVEPCRRRKEESDKNYFEGTGYARVPTQPHAPIPTFGQTIQTTVDRGLLFFAENGDRFISLNIEDGKLMVRYKLNSELPKERGVGDAINNGRDHSIQIKIGKLQKRMWINVDVQNTIIDGEVFDFSTYYLGGIPIAIRERFNISTPAFRGCMKNLKKTSGVVRLNDTVGVTKKCSEDWKLVRSASFSRGGQLSFTDLGLPPTDHLQASFGFQTFQPSGILLDHQTWTRNLQVTLEDGYIELSTSDSGGPIFKSPQTYMDGLLHYVSVISDNSGLRLLIDDQLLRNSKRLKHISSSRQSLRLGGSNFEGCISNVFVQRLSLSPEVLDLTSNSLKRDVSLGGCSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCLGGPLEKGIYFSEEGGHVVLAHSVLLGPEFKLVFSIRPRSLTGILIHIGSQPGKHLCVYLEAGKVTASMDSGAGGTSTSVTPKQSLCDGQWHSVAVTIKQHILHLELDTDSSYTAGQIPFPPASTQEPLHLGGAPANLTTLRIPVWKSFFGCLRNIHVNHIPVPVTEALEVQGPVSLNGCPDQ

SEQ ID NO: 9 - DNA, 3336 bp, Homo sapiens
atgcctgcgctctggctgggctgctgcctctgcttctcgctcctcctgcccgcagcccgggccacctccaggagggaagtctgtgattgcaatgggaagtccaggcagtgtatctttgatcgggaacttcacagacaaactggtaatggattccgctgcctcaactgcaatgacaacactgatggcattcactgcgagaagtgcaagaatggcttttaccggcacagagaaagggaccgctgtttgccctgcaattgtaactccaaaggttctcttagtgctcgatgtgacaactccggacggtgcagctgtaaaccaggtgtgacaggagccagatgcgaccgatgtctgccaggcttccacatgctcacggatgcggggtgcacccaagaccagagactgctagactccaagtgtgactgtgacccagctggcatcgcagggccctgtgacgcgggccgctgtgtctgcaagccagctgtcactggagaacgctgtgataggtgtcgatcaggttactataatctggatggggggaaccctgagggctgtacccagtgtttctgctatgggcattcagccagctgccgcagctctgcagaatacagtgtccataagatcacctctacctttcatcaagatgttgatggctggaaggctgtccaacgaaatgggtctcctgcaaagctccaatggtcacagcgccatcaagatgtgtttagctcagcccaacgactagaccctgtctattttgtggctcctgccaaatttcttgggaatcaacaggtgagctatggtcaaagcctgtcctttgactaccgtgtggacagaggaggcagacacccatctgcccatgatgtgattctggaaggtgctggtctacggatcacagctcccttgatgccacttggcaagacactgccttgtgggctcaccaagacttacacattcaggttaaatgagcatccaagcaataattggagcccccagctgagttactttgagtatcgaaggttactgcggaatctcacagccctccgcatccgagctacatatggagaatacagtactgggtacattgacaatgtgaccctgatttcagcccgccctgtctctggagccccagcaccctgggttgaacagtgtatatgtcctgttgggtacaaggggcaattctgccaggattgtgcttctggctacaagagagattcagcgagactggggccttttggcacctgtattccttgtaactgtcaagggggaggggcctgtgatccagacacaggagattgttattcaggggatgagaatcctgacattgagtgtgctgactgcccaattggtttctacaacgatccgcacgacccccgcagctgcaagccatgtccctgtcataacgggttcagctgctcagtgatgccggagacggaggaggtggtgtgcaataactgccctcccggggtcaccggtgcccgctgtgagctctgtgctgatggctactttggggacccctttggtgaacatggcccagtgaggccttgtcagccctgtcaatgcaacaacaatgtggaccccagtgcctctgggaattgtgaccggctgacaggcaggtgtttgaagtgtatccacaacacagccggcatctactgcgaccagtgcaaagcaggctacttcggggacccattggctcccaacccagcagacaagtgtcgagcttgcaactgtaaccccatgggctcagagcctgtaggatgtcgaagtgatggcacctgtgtttgcaagccaggatttggtggccccaactgtgagcatggagcattcagctgtccagcttgctataatcaagtgaagattcagatggatcagtttatgcagcagcttcagagaatggaggccctgatttcaaaggctcagggtggtgatggagtagtacctgatacagagctggaaggcaggatgcagcaggctgagcaggcccttcaggacattctgagagatgcccagatttcagaaggtgctagcagatcccttggtctccagttggccaaggtgaggagccaagagaacagctaccagagccgcctggatgacctcaagatgactgtggaaagagttcgggctctgggaagtcagtaccagaaccgagttcgggatactcacaggctcatcactcagatgcagctgagcctggcagaaagtgaagcttccttgggaaacactaacattcctgcctcagaccactacgtggggccaaatggctttaaaagtctggctcaggaggccacaagattagcagaaagccacgttgagtcagccagtaacatggagcaactgacaagggaaactgaggactattccaaacaagccctctcactggtgcgcaaggccctgcatgaaggagtcggaagcggaagcggtagcccggacggtgctgtggtgcaagggcttgtggaaaaattggagaaaaccaagtccctggcccagcagttgacaagggaggccactcaagcggaaattgaagcagataggtcttatcagcacagtctccgcctcctggattcagtgtctcggcttcagggagtcagtgatcagtcctttcaggtggaagaagcaaagaggatcaaacaaaaagcggattcactctcaagcctggtaaccaggcatatggatgagttcaagcgtacacagaagaatctgggaaactggaaagaagaagcacagcagctcttacagaatggaaaaagtgggagagagaaatcagatcagctgctttcccgtgccaatcttgctaaaagcagagcacaagaagcactgagtatgggcaatgccactttttatgaagttgagagcatccttaaaaacctcagagagtttgacctgcaggtggacaacagaaaagcagaagctgaagaagccatgaagagactctcctacatcagccagaaggtttcagatgccagtgacaagacccagcaagcagaaagagccctggggagcgctgctgctgatgcacagagggcaaagaatggggccggggaggccctggaaatctccagtgagattgaacaggagattgggagtctgaacttggaagccaatgtgacagcagatggagccttggccatggaaaagggactggcctctctgaagagtgagatgagggaagtggaaggagagctggaaaggaaggagctggagtttgacacgaatatggatgcagtacagatggtgattacagaagcccagaaggttgataccagagccaagaacgctggggttacaatccaagacacactcaacacattagacggcctcctgcatctgatgggtatgtga

SEQ ID NO: 10 - protein, 1111 aa, Homo sapiens
MPALWLGCCLCFSLLLPAARATSRREVCDCNGKSRQCIFDRELHRQTGNGFRCLNCNDNTDGIHCEKCKNGFYRHRERDRCLPCNCNSKGSLSARCDNSGRCSCKPGVTGARCDRCLPGFHMLTDAGCTQDQRLLDSKCDCDPAGIAGPCDAGRCVCKPAVTGERCDRCRSGYYNLDGGNPEGCTQCFCYGHSASCRSSAEYSVHKITSTFHQDVDGWKAVQRNGSPAKLQWSQRHQDVFSSAQRLDPVYFVAPAKFLGNQQVSYGQSLSFDYRVDRGGRHPSAHDVILEGAGLRITAPLMPLGKTLPCGLTKTYTFRLNEHPSNNWSPQLSYFEYRRLLRNLTALRIRATYGEYSTGYIDNVTLISARPVSGAPAPWVEQCICPVGYKGQFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFSCPACYNQVKIQMDQFMQQLQRMEALISKAQGGDGVVPDTELEGRMQQAEQALQDILRDAQISEGASRSLGLQLAKVRSQENSYQSRLDDLKMTVERVRALGSQYQNRVRDTHRLITQMQLSLAESEASLGNTNIPASDHYVGPNGFKSLAQEATRLAESHVESASNMEQLTRETEDYSKQALSLVRKALHEGVGSGSGSPDGAVVQGLVEKLEKTKSLAQQLTREATQAEIEADRSYQHSLRLLDSVSRLQGVSDQSFQVEEAKRIKQKADSLSSLVTRHMDEFKRTQKNLGNWKEEAQQLLQNGKSGREKSDQLLSRANLAKSRAQEALSMGNATFYEVESILKNLREFDLQVDNRKAEAEEAMKRLSYISQKVSDASDKTQQAERALGSAAADAQRAKNGAGEALEISSEIEQEIGSLNLEANVTADGALAMEKGLASLKSEMREVEGELERKELEFDTNMDAVQMVITEAQKVDTRAKNAGVTIQDTLNTLDGLLHLMGM

SEQ ID NO: 11 - DNA, 17 bp, Artificial Sequence
  oligo
tagtcccttaagcggag

SEQ ID NO: 12 - DNA, 46 bp, Artificial Sequence
  oligo
  misc_feature (23)..(28): n is a, c, g, or t
gtaatacgactcactatagggcnnnnnnctccgcttaagggactat

SEQ ID NO: 13 - DNA, 88 bp, Artificial Sequence
  Linker_primer_701_N
caagcagaagacggcatacgagatcgagtaatgtgactggagttcagacgtgtgctcttccgatctgtaatacgactcactatagggc

SEQ ID NO: 14 - DNA, 88 bp, Artificial Sequence
  Linker_primer_702_N
caagcagaagacggcatacgagattctccggagtgactggagttcagacgtgtgctcttccgatctgtaatacgactcactatagggc

SEQ ID NO: 15 - DNA, 88 bp, Artificial Sequence
  Linker_primer_703_N
caagcagaagacggcatacgagataatgagcggtgactggagttcagacgtgtgctcttccgatctgtaatacgactcactatagggc

SEQ ID NO: 16 - DNA, 95 bp, Artificial Sequence
  MuLV_LTR-3pIN_501_N
aatgatacggcgaccaccgagatctacactatagcctacactctttccctacacgacgctcttccgatctgacttgtggtctcgctgttccttgg

SEQ ID NO: 17 - DNA, 96 bp, Artificial Sequence
  MuLV_LTR-3pOUT_502_N
aatgatacggcgaccaccgagatctacacatagaggcacactctttccctacacgacgctcttccgatctgggtctcctctgagtgattgactacc

SEQ ID NO: 18 - DNA, 20 bp, Artificial Sequence
  oligonucleotide
ggtacccgtgtatccaataa

SEQ ID NO: 19 - DNA, 25 bp, Artificial Sequence
  oligonucleotide
gacttgtggtctcgctgttccttgg

SEQ ID NO: 20 - DNA, 25 bp, Artificial Sequence
  oligonucleotide
ggtctcctctgagtgattgactacc

SEQ ID NO: 21 - DNA, 20 bp, Artificial Sequence
  MLV 3′LTRlin_biotin primer
ggtacccgtgtatccaataa

SEQ ID NO: 22 - DNA, 25 bp, Artificial Sequence
  MLV 3′LTR_biotin primer
gacttgtggtctcgctgttccttgg

SEQ ID NO: 23 - DNA, 22 bp, Artificial Sequence
  LCrv primer
gtaatacgactcactatagggc

SEQ ID NO: 24 - DNA, 25 bp, Artificial Sequence
  MLV 3′LTR nested primer
ggtctcctctgagtgattgactacc

SEQ ID NO: 25 - DNA, 19 bp, Artificial Sequence
  LCrv primer
agggctccgcttaagggac

SEQ ID NO: 26 - DNA, 36 bp, Artificial Sequence
  LC1 TAlinkerMse(+) primer
gtaatacgactcactatagggctccgcttaagggac

SEQ ID NO: 27 - DNA, 17 bp, Artificial Sequence
  LC2 TAlinkerMse(-) primer
tagtcccttaagcggag

SEQ ID NO: 28 - DNA, 20 bp, Artificial Sequence
  primer
ggacctgaaatgaccctgtg

SEQ ID NO: 29 - DNA, 20 bp, Artificial Sequence
  MLV 3′LTR control F  primer
ggacctgaaatgaccctgtg

SEQ ID NO: 30 - DNA, 20 bp, Artificial Sequence
  Chr.5a primer
acccacagctcctgtctcat

SEQ ID NO: 31 - DNA, 21 bp, Artificial Sequence
  Chr.2a primer
ttctttcagtctggtggggtg

SEQ ID NO: 32 - DNA, 20 bp, Artificial Sequence
  Chr.4a primer
tggtggtggagtatctggag

SEQ ID NO: 33 - DNA, 21 bp, Artificial Sequence
  Chr.4b
gtggtggtggagtatctggag

SEQ ID NO: 34 - DNA, 20 bp, Artificial Sequence
  Chr.19a primer
ctcaccatcatgaggagcaa

SEQ ID NO: 35 - DNA, 20 bp, Artificial Sequence
  Chr.19b primer
ctcaccatcatgaggagcaa

SEQ ID NO: 36 - DNA, 21 bp, Artificial Sequence
  Chr.5b primer
gagcaatttgagggtcagaga

SEQ ID NO: 37 - DNA, 24 bp, Artificial Sequence
  primer
gaaatcaagattgtatcacgttcc

SEQ ID NO: 38 - DNA, 19 bp, Artificial Sequence
  primer
ctgcacacatgccctcttt

SEQ ID NO: 39 - DNA, 20 bp, Artificial Sequence
  primer
tcccaggaactttgttcaga

SEQ ID NO: 40 - DNA, 20 bp, Artificial Sequence
  primer
ccctaaggagctccaactga

SEQ ID NO: 41 - DNA, 20 bp, Artificial Sequence
  primer
ctgaggatggtggcagaaat

SEQ ID NO: 42 - DNA, 21 bp, Artificial Sequence
  primer
gccaattaacactcgttcacc

SEQ ID NO: 43 - DNA, 20 bp, Artificial Sequence
  primer
ggctcccaggtatgttctca

SEQ ID NO: 44 - DNA, 20 bp, Artificial Sequence
  primer
cctgatgttctgtcccccta

SEQ ID NO: 45 - DNA, 20 bp, Artificial Sequence
  primer
gcatgcacaacagctcaaac

SEQ ID NO: 46 - DNA, 21 bp, Artificial Sequence
  primer
gcctccatttggagagaaaat

SEQ ID NO: 47 - DNA, 20 bp, Artificial Sequence
  primer
cctcctcctcttcccttgat

SEQ ID NO: 48 - DNA, 20 bp, Artificial Sequence
  primer
cggcaaccactttaaaggac

SEQ ID NO: 49 - DNA, 25 bp, Artificial Sequence
  primer
gcctcactttctttctctgtaaatg

SEQ ID NO: 50 - DNA, 20 bp, Artificial Sequence
  primer
ggctcactgcaaccttcatc

SEQ ID NO: 51 - DNA, 21 bp, Artificial Sequence
  primer
ctggagctgggtgagataaag

SEQ ID NO: 52 - DNA, 20 bp, Artificial Sequence
  primer
ggaatggggcataagagaca

SEQ ID NO: 53 - DNA, 24 bp, Artificial Sequence
  primer
ttgagatagtcttacgctgtcacc

SEQ ID NO: 54 - DNA, 20 bp, Artificial Sequence
  primer
agtaacgccattttgcaagg

SEQ ID NO: 55 - DNA, 20 bp, Artificial Sequence
  primer
aacagaagcgagaagcgaac

SEQ ID NO: 56 - DNA, 20 bp, Artificial Sequence
  primer
agtaacgccattttgcaagg

SEQ ID NO: 57 - DNA, 20 bp, Artificial Sequence
  primer
aacagaagcgagaagcgaac